(19) United States
(12) Patent Application Publication
(10) Pub. No.: US 2018/0104257 A1
(43) Pub. Date: Apr. 19, 2018

(54) (17-ß)-3-Oxoandrost-4-En-17-Yl Undecanoate Compositions and Methods of Their Preparation and Use

(71) Applicant: Lipocine Inc., Salt Lake City, UT (US)

(72) Inventors: Mahesh V. Patel, Salt Lake City, UT (US); Nachiappan Chidambaram, Sandy, UT (US); Satish Kumar Nachaegari, Holladay, UT (US); Srinivansan Venkateshwaran, Salt Lake City, UT (US); Joel Frank, Sandy, UT (US); Amy Jo Portlock, Riverton, UT (US)

(73) Assignee: Lipocine Inc., Salt Lake City, UT (US)

(21) Appl. No.: 15/688,630

(22) Filed: Aug. 28, 2017

Related U.S. Application Data
(63) Continuation of application No. 14/841,583, filed on Aug. 31, 2015, now abandoned.
(60) Provisional application No. 62/043,877, filed on Aug. 29, 2014.

(51) Int. Cl. A61K 31/568; A61K 31/20; A61K 9/00; A61K 9/48; C07J 1/00; A61K 47/14

(52) U.S. Cl. CPC A61K 31/568; A61K 31/20; A61K 9/0053; A61K 2300/00; C07J 1/0025; A61K 47/14; A61K 9/4858

(57) ABSTRACT

Disclosed herein are compositions having a lipophilic active agent and methods of their use.

### CROSS-REFERENCE TO RELATED APPLICATIONS

This application is a continuation of U.S. patent application Ser. No. 14/841,583, filed Aug. 31, 2015, which claims the benefit of United States Provisional Application No. 62/043,877 filed Aug. 29, 2014, each of which is incorporated herein by reference.

### FIELD OF THE INVENTION

Disclosed herein are pharmaceutical compositions having or made from pure (17-β)-3-Oxoandrost-4-en-17-yl undecanoate for administration to mammals, e.g., humans, in need thereof.

### BACKGROUND

Worldwide marketing authorizations for pharmaceutical products set forth strict requirements for the stability and purity of pharmaceutical products regarding an active pharmaceutical ingredient (API) also known as drug or a prodrug of an API (also referred to herein as API) and its content. It is also critical that pure drug or prodrug is used in preparation of compositions or dosage forms of drugs or prodrugs to enable safe and effective use in treatment of appropriate disease conditions. Moreover, it is also important to limit all drug or prodrug related materials (starting impurities in the drug or prodrug, degradation product(s) derived from aging through chemical interaction between components of a composition or dosage form upon storage) to acceptable levels that are safe and do not limit shelf life of the composition or dosage form to an unacceptably short time.

A study recently found a number of (17-β)-hydroxy-4-androsten-3-one (also known as testosterone) replacement therapy products on the market that have problems with impurities (Baert el al. Volume 7 2, Issue 1, May 2009, Pages 275-281) (17-β)-3-Oxoandrost-4-en-17-yl undecanoate is an ester prodrug of (17-β)-hydroxy-4-androsten-3-one that has demonstrated promise in clinical testing in treating humans in need of (17-β)-hydroxy-4-androsten-3-one, a critical hormone useful for treating numerous conditions in males and females.

Due to its unique structure, (17-β)-3-Oxoandrost-4-en-17-yl undecanoate is particularly prone to decomposition as a result of reactions such as oxidation, reduction, cleavage of the ester moiety; oxidation of the steroid ring system; cleavage of one or more rings of the steroid ring system; rearrangement of the steroid ring; dealkylation of the steroid ring; dealkylation of the ester; or a combination thereof. Thus, the preparation and identification of substantially pure (17-β)-3-Oxoandrost-4-en-17-yl undecanoate and stable compositions and methods associated with use of stable (17-β)-3-Oxoandrost-4-en-17-yl undecanoate is challenging. Furthermore, (17-β)-3-Oxoandrost-4-en-17-yl undecanoate has a unique dosing regimen with a different unit dosage form and daily dose requirement for effectiveness as compared to conventional prodrugs of (17-β)-hydroxy-4-androsten-3-one reported to date. Therefore, a significant challenge that is unique to (17-β)-3-Oxoandrost-4-en-17-yl undecanoate relates to absolute levels of allowable impurities and degradation products for safe use in therapeutic settings.

Impurities in (17-β)-3-Oxoandrost-4-en-17-yl undecanoate can pose a number of problems to patients receiving (17-β)-3-Oxoandrost-4-en-17-yl undecanoate for therapy. Since (17-β)-3-Oxoandrost-4-en-17-yl undecanoate possesses a steroid core ring system, alteration of this molecule by decomposition or failure to remove synthetic by-products may result in exposing patients to potentially harmful agents. Compounds with steroid ring cores are often biologically active. For example, androstenedione (having a steroid core ring systemsimilar to that of (17-β)-hydroxy-4-androsten-3-one but having the hydroxyl group as a keto group) induces abnormalities in morphology and function of developing oocytes in female mice. (Fertil Steril. 2012 Feb;97(2):469-76, doi: 10.1016/j.fertnstert.2011.11.040) and androstenedione is carcinogenic in male and female mice Food Chem Toxicol. 2011 Sep;49(9):2116-24. doi: 10.1016/j.fct.2011.05.026. Epub 2011 May 30.

Thus, there is a need for substantially pure (17-β)-3-Oxoandrost-4-en-17-yl undecanoate and compositions (e.g., bulk drug substance, pharmaceutical, or unit dosage forms) containing substantially pure (17-β)-3-Oxoandrost-4-en-17-yl undecanoate, which can be stored for prolonged periods without deterioration of their quality, decrease in potency below a specified limit, as well as an increase of the concentration of decomposition products above acceptable limits. Moreover, there is even more critical need to prepare compositions and dosage forms that are stable upon storage for up to two years (or more) and are safe, with adequate potency (e.g., at least 80%) and acceptable (17-β)-3-Oxoandrost-4-en-17-yl undecanoate related substances in the compositions independent of using substantially pure starting (17-β)-3-Oxoandrost-4-en-17-yl undecanoate prodrug. (17-β)-3-Oxoandrost-4-en-17-yl undecanoate has the propensity to degrade or decompose. For example, (17-β)-3-Oxoandrost-4-en-17-yl undecanoate has the propensity to convert to (17-β)-Hydroxy-4-androsten-3-one (or otherwise decompose) due to hydrolysis upon storage or interaction with components of the composition or dosage forms that are acidic, hygroscopic, have an unsaturated moiety in their structure and contaminants or catalyst in the prodrug or excipients. Loss of potency of these compositions, especially those containing lipid additives (with associated free radicals) could compromise product performance.

Thus, there is a need for (17-β)-3-Oxoandrost-4-en-17-yl undecanoate products having acceptable levels of impurities, especially since this molecule has not yet received market authorization. Moreover, methods of stabilizing and inclusion of stabilizing agents that limit loss of potency of the compositions made with substantially (17-β)-3-Oxoandrost-4-en-17-yl undecanoate prodrug is particularly needed for compositions (and dosage forms) for safe and effective human us in need thereof. Ensuring safe levels of impurities and maintaining potency and stability of compounds like (17-β)-3-Oxoandrost-4-en-17-yl undecanoate is no small task given the daily dose requirements and the chronic therapeutic use of such agents.

### SUMMARY

Provided herein is substantially pure (17-β)-3-Oxoandrost-4-en-17-yl undecanoate pharmaceutical ingredient and compositions containing substantially pure (17-β)-3-Oxoandrost-4-en-17-yl undecanoate. In one embodiment, a substantially pure API or composition can have greater than 80% potency. Substantially pure (17-β)-3-Oxoandrost-4-en-17-yl undecanoate includes (17-β)-3-Oxoandrost-4-en-17-yl undecanoate substantially free of synthetic impurities, decomposition impurities, or both. In one embodiment an API or composition that is substantially free of impurities can have less than 20% of total impurities (known and unknown). The compositions of substantially pure (17-β)-3-Oxoandrost-4-en-17-yl undecanoate include substantially pure (17-β)-3-Oxoandrost-4-en-17-yl undecanoate pharmaceutical ingredient, pharmaceutical compositions comprised of or prepared from substantially pure (17-β)-3-Oxoandrost-4-en-17-yl undecanoate pharmaceutical ingredient and a pharmaceutically acceptable carrier, and unit dosage forms comprised of or prepared from substantially pure (17-β)-3-Oxoandrost-4-en-17-yl undecanoate pharmaceutical ingredient and a pharmaceutically acceptable carrier. Substantially pure (17-β)-3-Oxoandrost-4-en-17-yl undecanoate is useful for administration to a subject (e.g., mammalian; human) to provide safe and effective levels of 17-β)-3-Oxoandrost-4-en-17-yl undecanoate and (17-β)-hydroxy-4-androsten-3-one. For example, substantially pure (17-β)-3-Oxoandrost-4-en-17-yl undecanoate can be used to treat a subject having hypogonadism or any other disorder, disease or condition associated with low levelsof (17-β)-hydroxy-4-androsten-3-one or that can be improved or prevented (17-β)-hydroxy-4-androsen-3-one. Substantially pure (17-β)-3-Oxoandrost-4-en-17-yl undecanoate is useful in pharmaceutical applications and has exceptional safety characteristics as well as stability.

As is described herein, the present inventors have found that inclusion of specific carriers, additives or both, in pharmaceutical compositions or unit dosage forms provide adequate loading of (17-β)-3-Oxoandrost-4-en-17-yl undecanoate. These pharmaceutical compositions and unit dosage forms translate to a lower pill burden and improved, patient friendly, dosage regimes.

In one configuration, the composition and dosage form allow for advantageous loading of (17-β)-3-Oxoandrost-4-en-17-yl undecanoate (e.g., >23% w/w). The composition and dosage form can be formulated as a non-solid without any significant risk of precipitation upon storage (e.g., dissolution or release profile stable). The composition and dosage form can be formulated for suitable release that the inventors have found to be particularly suitable (e.g., at least 50% in two hours). The composition and dosage form provides adequate bioavailability (e.g., of (17-β)-3-Oxoandrost-4-en-17-yl undecanoate) while minimizing “pill burden” and avoiding complicated treatment regimens.

In one implementation, we have found that inclusion of an additive (e.g., long chain fatty acids (e.g., saturated) or fatty acid glycerides) in the pharmaceutical composition or dosage form enables advantageous loading of active pharmaceutical ingredient (“API”) (17-β)-3-Oxoandrost-4-en-17-yl undecanoate with suitable release (e.g., greater than 50% in two hours). The inclusion of a long chain fatty acid (e.g., saturated) or fatty acid glyceride can allow for loading of (17-β)-3-Oxoandrost-4-en-17-yl undecanoate greater than 23% w/w and no greater than 32% w/w in the compositions and dosage forms. The compositions and dosage forms can be formulated as a non-solid and provide one or more of the following: reduced, little or no risk of precipitation upon storage (e.g., dissolution or release profile stable); lower pill burden; patient friendly dosing regimen; appropriate releases or dissolution properties and suitable bioavailability. In this context, non-solid refers to a pharmaceutical composition that is not formulated or cannot be formulated as a tablet. At the same time, the pharmaceutical composition is not a liquid in the sense that the viscosity of the composition is increased over that of carrier alone or is a liquid at temperatures elevated above ambient (e.g., above 20-23° C.). In a specific implementation, the pharmaceutical is flowable at temperatures that allow for the filling of soft gel capsules. At certain temperatures e.g., below ambient or below 15 or 10° C. the composition is more solid like and is not flowable (e.g., for filling soft gel capsules). These compositions can be used for hard gel or soft gel capsules. These compositions comprise a liquid pharmaceutically acceptable carrier and an additive that allows for loading of the API above the solubility of the limit of the liquid carrier.

In another configuration, the present inventors have found that inclusion of stabilizing agent in the composition and dosage form enables advantageous loading of (17-β)-3-Oxoandrost-4-en-17-yl undecanoate. The stabilizing agent allows for valuable loading of (17-β)-3-Oxoandrost-4-en-17-yl undecanoate (e.g., >23% w/w). The stabilizing agent allows the composition and dosage form to be formulated as a non-liquid and provides one or more of the following: reduced, little or no risk of precipitation upon storage (e.g., dissolution or release profile stable); lower pill burden; patient friendly dosing regimen; and suitable bioavailability. In this context, non-liquid refers to a pharmaceutical composition that is not a liquid in the sense that the viscosity of the composition at ambient temperature (e.g., above 20-23° C.). In a specific implementation, the pharmaceutical is flowable at temperatures that allow for the filling of hard gel capsules. At certain temperatures e.g., at ambient or below 15 or 10° C. the composition is more solid like and is not flowable (e.g., for filling soft gel capsules). In one aspect, these compositions can be used for hard gel capsules. These compositions comprise a liquid pharmaceutically acceptable carrier and a stabilizing agent that allows for loading of the API above the solubility of the limit of the liquid carrier.

In yet another implementation, we have found that inclusion of stabilizing agent in the composition and dosage form enables advantageous loading of (17-β)-3-Oxoandrost-4-en-17-yl undecanoate with adequate release properties (e.g., greater than 50% in 2 hours). The stabilizing agent allows the composition and dosage form to be formulated as a non-liquid and provides one or more of the following: lower daily pill burden; valuable loading e.g., greater than 23% w/w and no greater than 32% w/w); reduced, little or no risk of precipitation upon storage (e.g., dissolution or release profile stable); adequate bioavailability; and patient friendly dosing regimen.

As described herein, the inventors have discovered compositions and dosages forms that, in some embodiments, allow for unexpectedly high drug loading for highly lipophilic drugs while maintaining excellent oral bioavailability. The pharmaceutical compositions and unit dosage forms described herein can reduce pill burden for hydrophobic drugs like (17-β)-3-Oxoandrost-4-en-17-yl undecanoate. It was unexpectedly found that (17-β)-3-Oxoandrost-4-en-17-yl undecanoate can be formulated at advantageous drug loads (e.g., greater than 23% w/w) while providing suitable bioavailability (e.g., capable of treating a hypogonadal male with less than 10 unit dosage forms per day) that allows for reduction in pill burden and accordingly improved patient adherence or compliance. Additionally, the composition (e.g., dosage form) has a release profile that is suitable for providing bioavailable API and the release profile is stable over time (e.g., under storage conditions).

According to one implementation, the pharmaceutical composition is a liquid (e.g., flowable) or the unit dosage form comprises a liquid pharmaceutical composition. For example, the unit dosage form can be a hard gel or soft gel capsule which contains a liquid pharmaceutical carrier in which the drug is dissolved (partially or fully). In a specific context, flowable refers to the ability of the composition to flow at a temperature of 40° C. With respect to liquid compositions, in one context, the liquid pharmaceutical carrier (like a C18 fatty acid having one unsaturation) is a liquid at ambient temperature (e.g., about 20 to about 25° C.). Typically, the liquid pharmaceutical compositions also include one or more additives that allow for increased loading of active pharmaceutical ingredient in the carrier beyond its solubility without substantial compromising dissolution (e.g., release), bioavailability or both. The one or more additives in specific aspects can increase the viscosity of the pharmaceutical composition. In specific configurations, the liquid pharmaceutical composition (e.g., carrier +additive +API (active pharmaceutical ingredient) is a clear liquid at elevated temperatures (e.g., above 40° C., like 50° C.) flowable in the range of about 30° C. to about 40° C. and may be more hazy in appearance at ambient temperature having a paste or gel like consistency. In another implementation, the pharmaceutical composition is a liquid or the unit dosage form comprises a liquid. For example, the unit dosage form can be a hard gel or soft gel capsule which contains a liquid pharmaceutical carrier in which the drug is dissolved along with one or more additives. In a specific implementation, the unit dosage form is a soft gel capsule. According to a soft gel capsule implementation, the formulation (e.g., mixture of one or more pharmaceutical acceptable carriers and active pharmaceutical ingredient) is flowable at a temperature of less than 40° C. Exemplary carriers include fatty acids (e.g., C16-C18 having zero, one, two, or three unsaturations) and mono-, di-, and triglycerides thereof). According to this implementation, the composition or dosage form comprises (17-β)-3-Oxoandrost-4-en-17-yl undecanoate.

According to another implementation, the pharmaceutical composition is a non-liquid (e.g., flowable) or the unit dosage form comprises a non-liquid pharmaceutical composition. For example, the unit dosage form can be a hard gel capsule which contains a liquid pharmaceutical carrier in which the drug is dissolved (partially or fully). In a specific context, flowable refers to the ability of the composition to flow at a temperature of 40° C. With respect to non-liquid compositions, in one context, pharmaceutical carrier (e.g., fatty acid, fatty acid glyceride, or combination thereof) is a liquid or solid at ambient temperature (e.g., about 20 to about 25° C.). Typically, the non-liquid pharmaceutical compositions also include one or more additives that allow for increased loading of active pharmaceutical ingredient in the carrier beyond its solubility without substantial compromising dissolution (e.g., release), bioavailability or both. The one or more additives in specific aspects can increase the viscosity of the pharmaceutical composition. In specific configurations, the non-liquid pharmaceutical composition (e.g., carrier+additive+API (active pharmaceutical ingredient) is a clear liquid at elevated temperatures (e.g., above 40° C., like 50° C.) flowable in the range of about 30° C. to about 40° C. and may be more hazy in appearance at ambient temperature having a paste or gel like consistency. In another implementation, the pharmaceutical composition is a non-liquid or the unit dosage form comprises a non-liquid. For example, the unit dosage form can be a hard gel capsule which contains a non-liquid pharmaceutical carrier in which the drug is dissolved along with one or more additives. In a specific implementation, the unit dosage form is a hard gel capsule. According to a hard gel capsule implementation, the formulation (e.g., mixture of one or more pharmaceutical acceptable carriers and active pharmaceutical ingredient) is flowable at a temperature of greater than 35 or 40° C. and is not flowable at temperatures below 25° C. (e.g., not suitable for capsule filling at this temperature). Exemplary carriers include fatty acids (e.g., C16-C18 having zero, one, two, or three unsaturations) and mono-, di-, and triglycerides thereof). According to this implementation, the composition or dosage form comprises (17-β)-3-Oxoandrost-4-en-17-yl undecanoate.

A specific example composition comprises a liquid or liquid-like carrier and an additive as well as API. The carrier is a liquid at ambient temperature in which API has sufficient solubility and the additive increases the amount of API you can get into formulation without substantially compromising bioavailability or release. In a particular example, the composition comprises (a) octadecanoic acid, (9Z)-octadec-9-enoic acid, hexadecanoic acid, or a combination thereof (b) a mono-, di-, tri-propane-1,2,3-triol ester thereof or a combination thereof or (c) a combination thereof where the API is loaded in the range of 26% to 35% w/w or 26% to 32% w/w. In another particular example, the compositions comprises (a) octadecanoic acid, (9Z)-octadec-9-enoic acid, hexadecanoic acid or a combination thereof (b) a mono-, di-, tri-propane-1,2,3-triol ester thereof or a combination thereof, (c) 2-Isopropyl-5-methylcyclohexanone or (d) a combination thereof where the API is loaded in the range of 26% to 35% w/w or 26% to 32% w/w.

Another specific example composition comprises a carrier that is a liquid at ambient temperature (e.g., 20-23° C.) in which the API has sufficient solubility and an additive that prevents crystallization or recrystallization of API. In a particular example, the carrier comprises (9Z)-octadec-9-enoic acid and the compound of formula H—(O—CH₂—CH₂)ₙ—OH characterized as having an average molecular weight of about 600 to about 15,000 gram/mol where n is defined as giving said average molecular weight where the API is loaded in the range of 26% to 35% w/w or 26% to 32% w/w.

These specific compositions allow for delivery of 300 to 1500 mgs of API in 2-6 unit dosage forms (hard gel or soft gel capsules) per day. Lower number of unit dosage form are provided with soft gel capsules which is a reflection of additional innovation because of requirement of flowability of the composition at temperatures that allow filing (e.g., at 38° C. or less). These compositions provide excellent bioavailability for testosterone—see examples. The unit dosage forms are dissolution (release) profile stable over time (single point or profile), releases greater than 70% drug by 4 hours and greater than 50% at 2 hours.

### BRIEF DESCRIPTION OF THE DRAWINGS

FIG. 1A shows a non-limiting example of a synthetic scheme for (17-β)-3-Oxoandrost-4-en-17-yl undecanoate.

FIG. 1B shows non-limiting examples of crystallization of (17-β)-3-Oxoandrost-4-en-17-yl undecanoate.

FIG. 2 shows general classification of potential impurities in (17-β)-3-Oxoandrost-4-en-17-yl undecanoate samples.

FIG. 3 shows a classification of potential impurities in (17-β)-3-Oxoandrost-4-en-17-yl undecanoate samples.

FIG. 4 shows a classification of potential impurities related to (17-β)-3-Oxoandrost-4-en-17-yl undecanoate samples.

FIG. 5 shows a classification of potential impurities related to (17-β)-3-Oxoandrost-4-en-17-yl undecanoate samples.

FIG. 6 shows a classification of potential impurities related to (17-β)-3-Oxoandrost-4-en-17-yl undecanoate.

FIG. 7 shows the structures of several potential impurities related to (17-β)-3-Oxoandrost-4-en-17-yl undecanoate

FIG. 8 shows the structures of several potential impurities related to (17-β)-3-Oxoandrost-4-en-17-yl undecanoate.

FIG. 9 shows the structures of several potential impurities related to (17-β)-3-Oxoandrost-4-en-17-yl undecanoate that underwent forced degradation by HPLC. The arrows show peaks related to top compounds in FIG. 7 and other related compounds are also listed.

FIG. 10 shows the structures of impurities related to (17-β)-3-Oxoandrost-4-en-17-yl undecanoate that underwent forced degradation as assessed by HPLC. Some of these impurities are listed herein.

FIG. 11 shows the release profile stability of a pharmaceutical composition having (17-β)-3-Oxoandrost-4-en-17-yl undecanoate. See Example 8.

FIG. 12 shows the release profile stability of a pharmaceutical composition having (17-β)-3-Oxoandrost-4-en-17-yl undecanoate. See Example 8.

### DETAILED DESCRIPTION OF THE INVENTION

As described herein, substantially pure compositions of (17-β)-3-Oxoandrost-4-en-17-yl undecanoate are provided that are free of synthetic impurities, decomposition impurities, or both. The substantially pure compositions of (17-β)-3-Oxoandrost-4-en-17-yl undecanoate described herein include bulk prodrug, pharmaceutical compositions having substantially pure (17-β)-3-Oxoandrost-4-en-17-yl undecanoate and unit dosage forms having substantially pure (17-β)-3-Oxoandrost-4-en-17-yl undecanoate. The compositions described herein may further include a stabilizing agent or are stored under stabilizing conditions. Additionally, methods of using substantially pure compositions of (17-β)-3-Oxoandrost-4-en-17-yl undecanoate are described, including methods of treatment (e.g., treating a disease, disorder or condition in an individual), and methods of producing or preparing compositions. The pharmaceutical compositions described herein have substantially pure (17-β)-3-Oxoandrost-4-en-17-yl undecanoate and a pharmaceutically acceptable carrier. Unit dosage forms can be formed from the pharmaceutical compositions e.g., tablet or capsule (soft gel or hard gel).

Formulations having advantageous loading lipophilic active pharmaceutical ingredient (17-β)-3-Oxoandrost-4-en-17-yl undecanoate are provided herein. The formulations having advantageous loading of drug described herein include pharmaceutical compositions, unit dosage forms having the pharmaceutical composition and intermediate compositions in the production of the high drug load formulations, pharmaceutical compositions and unit dosage forms described herein. The pharmaceutical compositions and unit dosage forms described herein are not solid. “Not Solid” refers to a pharmaceutical composition that is flowable, semi-solid, a liquid, past, gel or liquid-like either at ambient temperature or processing temperatures that are suitable for hard gel of soft gel filling yet do not result in substantial degradation or decomposition of the active agent. “Solid” in this context refers to a composition that is a tablet (or caplet) or that can be formed into a tablet with acceptable characteristics (e.g., friability, hardness and disintegration).

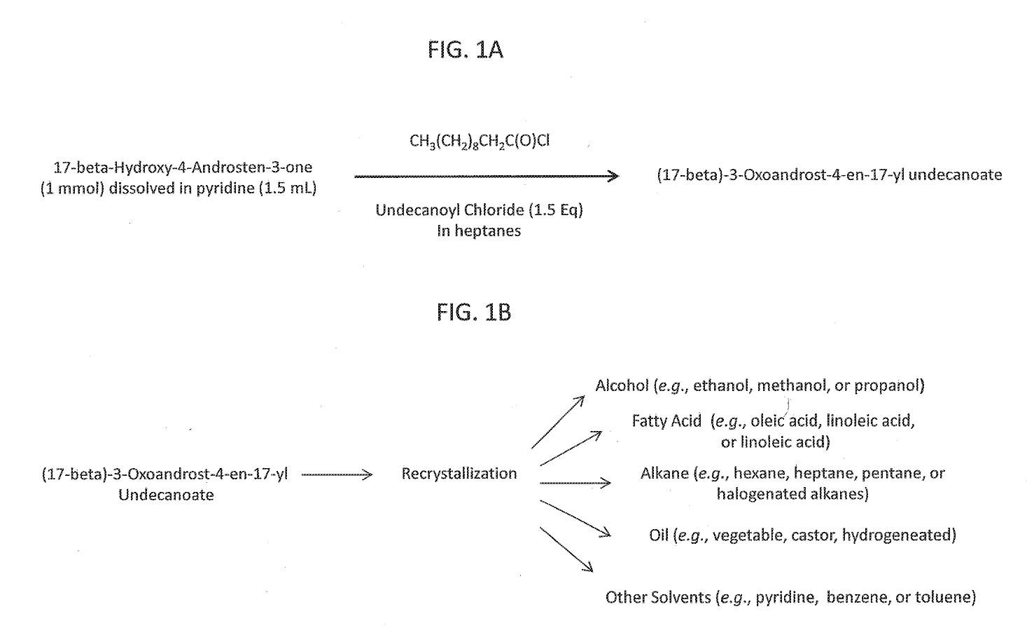

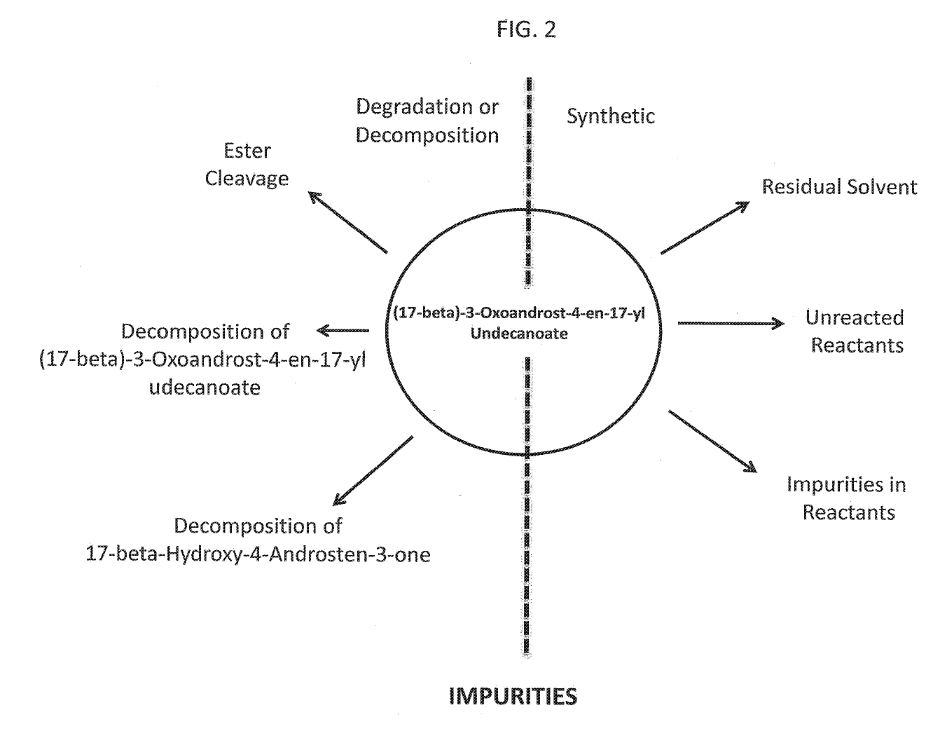

As described herein, the inventors have surprisingly found that advantageous drug load formulations of (17-β)-3-Oxoandrost-4-en-17-yl undecanoate can be made that have excellent dissolution properties including release of substantially all of the active agent within 4 hours and are dissolution profile stable over a period of time. For example, FIG. 1 and FIG. 2 show formulations that release substantially all of the active agent at four hours (e.g., greater than 90% or 95%). Formulation types that do not release substantially all of the active agent at four hours are described e.g., have amounts of controlled release agent (e.g., 15%+HPMC (e.g., hypromellose 100 cP (K100) or 4000 cP (K4M) agents and other excipients at levels where they substantially retard release.

It was unexpectedly found that liquid carriers can have improved drug loading (e.g., greater than the solubility of the drug in the liquid carrier) when formulated in a liquid carrier having one or more additives without substantially compromising dissolution or bioavailability. In one specific embodiment, the formulations or compositions (e.g., active agent+liquid carrier+one or more additives) are flowable at temperatures (e.g., 40° C. or less) that allow for production (e.g., filling) of soft gel capsule unit dosage forms without substantially comprising the structural integrity of the capsule or API. It appears that the combination of carriers, additives, and API provide some synergistic or unexpected effect that allows for loading above the solubility of the API in the carrier and provides suitable bioavailability.

Definitions

It should be noted that, the singular forms “a,” “an,” and, “the” include plural references unless the context clearly dictates otherwise. Thus, for example, reference to “an excipient” includes reference to one or more of such excipients, and reference to “the carrier” includes reference to one or more of such carriers.

As used herein, “active pharmaceutical ingredient” refers to (17-β)-3-Oxoandrost-4-en-17-yl undecanoate. The biologically active metabolite of (17-β)-3-Oxoandrost-4-en-17-yl undecanoate is (17-β)-Hydroxy-4-Androsten-3-one which is produced in vivo by de-esterification. Another important biologically active metabolite is (17-β)-hydroxy-5α-androstan-3-one with an IUPAC name of (5S,8R,9S,10S,13 S,14S,17S)-17-hydroxy-10,13-dimethyl-1,2,4,5,6,7,8,9,11,12,14,15,16,17-tetradecahydrocyclopenta[a]phenanthren-3-one (CAS No. 521-18-6).

As used herein, the term “(17-β)-Hydroxy-4-Androsten-3-one” refers to a chemical having an IUPAC name of (8R,9S,10R,13S, 14S,17S)-17-Hydroxy-10,13-dimethyl-1,2,6,7,8,9,11,12,14,15,16,17-dodecahydrocyclopenta[a]phenanthren-3-one and a CAS number of 58-22-0. (17-β)-3-Oxoandrost-4-en-17-yl undecanoate refers to a compound having the (17-β)-Hydroxy-4-Androsten-3-one core structure but the hydroxyl group is esterified with undecanoic acid e.g., (17-β)-Hydroxy-4-Androsten-3-one esterified with a straight chain saturated 11 carbon long alkanoic acid called undecanoic acid. Undecanoic acid is the IUPAC name for the alkanoic acid having CAS number 112-37-8.

As used herein, “impurity” or “impurities” refer to a chemical or chemical that is not (17-β)-3-Oxoandrost-4-en-17-yl undecanoate but is derived from the synthesis, preparation, processing, degradation or decomposition of (17-β)-3-Oxoandrost-4-en-17-yl undecanoate. Typically, the impurity is a synthetic impurity or a decomposition impurity. Impurities typically can be derived from decomposition, degradation or the chemical reaction of (17-β)-3-Oxoandrost-4-en-17-yl undecanoate which can be referred to as related compounds or “RC” or “RCs”. Other impurities can be present as described herein and are typically synthetic by-products, solvents, etc., derived from the manufacturing or processing of the API. Although synthetic and decomposition impurities are used to categorize classes described herein, they are not mutually exclusive and it is not always certain where or how a particular impurity arises.

As used herein, “substantially pure” refers to a composition having an active pharmaceutical ingredient which meets applicable regulatory requirements in terms of potency. In this context, potency refers to the comparison of a reference standard deemed to be 100% potent by any number of techniques including NMR, elemental analysis, IR, chromatography (e.g., HPLC) and the such. In one definition, potency is defined in terms of the definition provided by a compendium e.g., the United States Pharmacopeia, European Pharmacopeia or other national or regional Pharmacopeia. Potency can be determined by one of ordinary skill in the art in view of the definition in the compendium or as described herein. In one embodiment, the substantially pure (17-β)-3-Oxoandrost-4-en-17-yl undecanoate or related compositions has greater than 80%, 85%, 90%, 95%, 96%, 97%, 98% or 99% potency.

As used herein, “substantially free of impurities” refers to a composition having active pharmaceutical ingredient which meets applicable regulatory requirements for levels of impurities (e.g., below a specific level). Depending on the context, substantially free of impurities can refer to all impurities or a specific impurity which is 50% or less, 40% or less, 30% or less, 20% or less, 10% or less, 5% or less, 3% or less, 2% or less, 1% or less, 0.5% or less, 0.3% or less, 0.2% or less, or 0.1% or less by total weight of active pharmaceutical ingredient and the specific impurity or impurities (or total impurities). Unless otherwise specified percent impurity is calculated as the (total weight of a specific impurity or impurities)/(total weight of API+weight of specific impurity or impurities)*100. In some instances, other methods beside weight are used to characterize impurities, like area under the curves of HPLC traces or NMR signals (e.g., via integration) which can be used to calculate percent impurities also. The impurities can be decomposition impurities, synthetic impurities, or any other impurity. The synthetic impurities, in some aspects, refers to those identified in the examples, figures, or elsewhere herein.

As used herein, “substantially free of synthetic impurities” refers to a composition having (17-β)-3-Oxoandrost-4-en-17-yl undecanoate which meets applicable regulatory requirements for levels of impurities (e.g., below a specific level) where the impurity is related to synthesis and/or processing of(17-β)-3-Oxoandrost-4-en-17-yl undecanoate. Depending on the context, substantially free of synthetic impurities can refer to all synthetic impurities or a specific synthetic impurity which is 50% or less, 40% or less, 30% or less, 20% or less, 10% or less, 5% or less, 3% or less, 2% or less, 1% or less, 0.5% or less, 0.3% or less, 0.2% or less, or 0.1% or less by total weight of (17-β)-3-Oxoandrost-4-en-17-yl undecanoate and the specific synthetic impurity or impurities. The synthetic impurities, in some aspects, refer to those identified in the examples, figures, or elsewhere herein. Synthetic impurities of (17-β)-3-Oxoandrost-4-en-17-yl undecanoate include, but are not limited to, starting materials, by-products (from reactions of reagents or other materials), side-products (from reaction of impurities in reagents or other materials), intermediates, degradation (or decomposition) products, reagents, ligands, catalysts, residual solvents (e.g., from the synthesis, purification or processing of API). Specific synthetic impurities include, but are not limited to, 17-β-Hydroxy-4-Androsten-3-one, undecanoic acid (or a salt form thereof), undecanoyl chloride, etc., or a non-API compound derived therefrom, pyridine, heptane, heptanes, etc.

It is noted that the term stabilizing agent can refer to a compound like ascorbyl palmitate that prevents or slows the formulation of API related or derived impurities or in other contexts is a formulation ingredient that aids in loading of API e.g., prevents crystallization etc.

As used herein, “substantially free of decomposition impurities” refers to a composition having (17-β)-3-Oxoandrost-4-en-17-yl undecanoate which meets applicable regulatory requirements for levels of impurities related to the decomposition (including degradation) of (17-β)-3-Oxoandrost-4-en-17-yl undecanoate (e.g., below a specific level). Depending on the context, free of decomposition impurities can refer to all decomposition impurities or a specific synthetic impurity which is 50% or less, 40% or less, 30% or less, 20% or less, 10% or less, 5% or less, 3% or less, 2% or less, 1% or less, 0.5% or less, 0.3% or less, 0.2% or less, or 0.1% or less by total weight of (17-β)-3-Oxoandrost-4-en-17-yl undecanoate and the specific or known decomposition impurity or unknown or unspecified impurities. The decomposition impurities, in some aspects, refer to those identified in the examples, figures, or elsewhere herein. Decomposition impurities include, but are not limited to (17-β)-hydroxy-4-androsten-3-one, an oxidation product of (17-β)-3-Oxoandrost-4-en-17-yl undecanoate, an oxidation product of 17-β-Hydroxy-4-Androsten-3-one, or other decomposition products.

In one embodiment, the impurity is 3-Oxoandrost-1,4-dien-17β-yl undecanoate, 3-Oxoandrost-4,6-dien-17β-yl undecanoate, 17 beta-hydroxy androst-4-en-3-one tridecylenate (e.g., tridec-12-enoate), 3-Oxoandrost-4-en-17α-yl undecanoate , 4-Androstene-3,17-dione, 17α-hydroxyandrost-4-en-3one, 4-Androstene-3β, 17β-diol, androsta-1,4-dien-3,17-dione, 17β-hydroxyandrosta-4,6-dien-3-one, 17β-hydroxy-5α-androstan-3-one, or 17β-hydroxyandrost-1,4-dien-3-one.

In one embodiment, the impurity is as shown in any of the Figures.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

Provided herein are compositions containing substantially pure (17-β)-3-Oxoandrost-4-en-17-yl undecanoate. The substantially pure (17-β)-3-Oxoandrost-4-en-17-yl undecanoate can be substantially free of impurities. Impurities of compositions containing (17-β)-3-Oxoandrost-4-en-17-yl undecanoate include, but are not limited to, synthetic impurities, decomposition impurities, or both. In one aspect, the substantially pure (17-β)-3-Oxoandrost-4-en-17-yl undecanoate is substantially free of impurities specifically disclosed herein or in any of the Figures.

In one embodiment, a substantially pure (17-β)-3-Oxoandrost-4-en-17-yl undecanoate composition is provided that is at least 80%, 85% or 90% substantially pure (17β)-3-Oxoandrost-4-en-17-yl undecanoate. According to an aspect of this embodiment, the composition comprises at least 95% substantially pure (17-β)-3-Oxoandrost-4-en-17-yl undecanoate. In a more specific aspect, the composition comprises at least 97% substantially pure (17-β)-3-Oxoandrost-4-en-17-yl undecanoate. In another more specific aspect, the composition comprises at least 98% substantially pure (17-β)-3-Oxoandrost-4-en-17-yl undecanoate. In another more specific aspect, the composition comprises at least 99% substantially pure (17-β)-3-Oxoandrost-4-en-17-yl undecanoate. In another more specific aspect, the composition comprises at least 99.5% substantially pure (17-β)-3-Oxoandrost-4-en-17-yl undecanoate. In a specific aspect, the substantially pure (17-β)-3-Oxoandrost-4-en-17-yl undecanoate is substantially free of impurities. In one embodiment, a substantially pure (17-β)-3-Oxoandrost-4-en-17-yl undecanoate is a white crystalline or amorphous powder. In one aspect, at least 1 g, 10g, 50 g, 100 g, 500 g, 1 kg, 5 kg, 10 kg, 50 kg, 100 kg, 500 kg, 1000 kg, 5000 kg 10,000 kg, 50,000 kg of substantially pure (17-β)-3-Oxoandrost-4-en-17-yl undecanoate is provided. In one aspect, the substantially pure (17-β)-3-Oxoandrost-4-en-17-yl undecanoate is substantially free of impurities specifically disclosed herein or in any of the Figures.

In another embodiment, a pharmaceutical composition is provided which comprises or is prepared from (17-β)-3-Oxoandrost-4-en-17-yl undecanoate that is at least 80%, 85% or 90% substantially pure (17-β)-3-Oxoandrost-4-en-17-yl undecanoate and a pharmaceutically acceptable carrier. According to an aspect of this embodiment, the pharmaceutical composition comprises or is prepared from (17-β)-3-Oxoandrost-4-en-17-yl undecanoate that is at least 95% substantially pure (17-β)-3-Oxoandrost-4-en-17-yl undecanoate and a pharmaceutically acceptable carrier. In a more specific aspect, the pharmaceutical composition comprises or is prepared from (17-β)-3-Oxoandrost-4-en-17-yl undecanoate that is at least 97% substantially pure (17-β)-3-Oxoandrost-4-en-17-yl undecanoate and a pharmaceutically acceptable carrier. In another more specific aspect, the pharmaceutical composition comprises or is prepared from (17-β)-3-Oxoandrost-4-en-17-yl undecanoate that is at least 98% substantially pure (17-β)-3-Oxoandrost-4-en-17-yl undecanoate and a pharmaceutically acceptable carrier. In another more specific aspect, the pharmaceutical composition comprises or is prepared from (17-β)-3-Oxoandrost-4-en-17-yl undecanoate that is at least 99% substantially pure (17-β)-3-Oxoandrost-4-en-17-yl undecanoate and a pharmaceutically acceptable carrier. In another more specific aspect, the pharmaceutical composition comprises or is prepared from (17-β)-3-Oxoandrost-4-en-17-yl undecanoate that is at least 99.5% substantially pure (17-β)-3-Oxoandrost-4-en-17-yl undecanoate and a pharmaceutically acceptable carrier. In a specific aspect, the pharmaceutical composition comprises or is prepared from substantially pure (17-β)-3-Oxoandrost-4-en-17-yl undecanoate that is a white crystalline or amorphous powder. Pharmaceutical compositions of this embodiment are suitable for any form of administration. For example, the pharmaceutical formulations can be formulated for enteral, parenteral, or topical administration. In one aspect, the substantially pure (17-β)-3-Oxoandrost-4-en-17-yl undecanoate is substantially free of impurities. In one aspect, the substantially pure (17-β)-3-Oxoandrost-4-en-17-yl undecanoate is substantially free of impurities specifically disclosed herein or in any of the Figures.

In yet another embodiment, a unit dosage form is provided which comprises or is prepared from (17-β)-3-Oxoandrost-4-en-17-yl undecanoate that is at least 80%, 85% or 90% substantially pure (17-β)-3-Oxoandrost-4-en-17-yl undecanoate and a pharmaceutically acceptable carrier.

According to an aspect of this embodiment, the unit dosage form comprises or is prepared from (17-β)-3-Oxoandrost-4-en-17-yl undecanoate and that is at least 95% substantially pure (17-β)-3-Oxoandrost-4-en-17-yl undecanoate and a pharmaceutically acceptable carrier. In a more specific aspect, the unit dosage form comprises or is prepared from substantially pure (17-β)-3-Oxoandrost-4-en-17-yl undecanoate that is at least 97% substantially pure (17-β)-3-Oxoandrost-4-en-17-yl undecanoate and a pharmaceutically acceptable carrier. In another more specific aspect, the unit dosage form comprises or is prepared from (17-β)-3-Oxoandrost-4-en-17-yl undecanoate that is at least 98% substantially pure (17-β)-3-Oxoandrost-4-en-17-yl undecanoate and a pharmaceutically acceptable carrier. In another more specific aspect, the unit dosage form comprises or is prepared from (17-β)-3-Oxoandrost-4-en-17-yl undecanoate that is at least 99% substantially pure (17-β)-3-Oxoandrost-4-en-17-yl undecanoate and a pharmaceutically acceptable carrier. In another more specific aspect, the unit dosage form comprises or is prepared from (17-β)-3-Oxoandrost-4-en-17-yl undecanoate that is at least 99.5% substantially pure (17-β)-3-Oxoandrost-4-en-17-yl undecanoate and a pharmaceutically acceptable carrier. In a specific aspect, the unit dosage form comprises or is prepared from pharmaceutical ingredient, (17-β)-3-Oxoandrost-4-en-17-yl undecanoate that is a white crystalline or amorphous powder. Unit dosage forms of this embodiment are suitable for any form of administration. For example, the unit dosage form of the compositions of this invention can be formulated for enteral, parenteral, or topical administration. In one aspect, the substantially pure (17-β)-3-Oxoandrost-4-en-17-yl undecanoate is substantially free of impurities. In one aspect, the substantially pure (17-β)-3-Oxoandrost-4-en-17-yl undecanoate is substantially free of impurities specifically disclosed herein or in any of the Figures.

In one embodiment, a substantially pure (17-β)-3-Oxoandrost-4-en-17-yl undecanoate composition is provided. According to this embodiment, the composition comprises at least 95% (17-β)-3-Oxoandrost-4-en-17-yl undecanoate. In a more specific aspect, the composition comprises at least 97% (17-β)-3-Oxoandrost-4-en-17-yl undecanoate. In another more specific aspect, the composition comprises at least 98% (17-β)-3-Oxoandrost-4-en-17-yl undecanoate. In another more specific aspect, the composition comprises at least 99% (17-β)-3-Oxoandrost-4-en-17-yl undecanoate. In another more specific aspect, the composition comprises at least 99.5% (17-β)-3-Oxoandrost-4-en-17-yl undecanoate. In a specific aspect, the active pharmaceutical ingredient is a white crystalline or amorphous powder. According to one aspect of this embodiment, the substantially pure (17-β)-3-Oxoandrost-4-en-17-yl undecanoate comprises less than 20%, 15% or 10% impurities (e.g., total impurities (known +unknown). In a more specific aspect, the (17-β)-3-Oxoandrost-4-en-17-yl undecanoate comprises less than 5% total impurities. In another more specific aspect, the (17-β)-3-Oxoandrost-4-en-17-yl undecanoate comprises less than 3% total impurities. In another more specific aspect, the (17-β)-3-Oxoandrost-4-en-17-yl undecanoate comprises less than 2% total impurities. In another more specific aspect, the (17-β)-3-Oxoandrost-4-en-17-yl undecanoate comprises less than 1% total impurities. In another more specific aspect, the (17-β)-3-Oxoandrost-4-en-17-yl undecanoate comprises less than 5% 17-β-Hydroxy-4-Androsten-3-one. In another more specific aspect, the (17-β)-3-Oxoandrost-4-en-17-yl undecanoate comprises less than 3% 17-β-Hydroxy-4-Androsten-3-one. In another more specific aspect, the (17-β)-3-Oxoandrost-4-en-17-yl undecanoate comprises less than 2% 17-β-Hydroxy-4-Androsten-3-one. In another more specific aspect, the (17-β)-3-Oxoandrost-4-en-17-yl undecanoate comprises less than 1% 17-β-Hydroxy-4-Androsten-3-one. In another more specific aspect, the (17-β)-3-Oxoandrost-4-en-17-yl undecanoate comprises less than 0.5% 17-β-Hydroxy-4-Androsten-3-one. In another specific aspect, the (17-β)-3-Oxoandrost-4-en-17-yl undecanoate comprises less than 20000 ppm of a solvent (e.g., ethanol). In another more specific aspect, the (17-β)-3-Oxoandrost-4-en-17-yl undecanoate comprises less than 10000 ppm of a solvent (e.g., ethanol). In another more specific aspect, the (17-β)-3-Oxoandrost-4-en-17-yl undecanoate comprises less than 5000 ppm of a solvent (e.g., ethanol). In another more specific aspect, the (17-β)-3-Oxoandrost-4-en-17-yl undecanoate comprises less than 3000 ppm of a solvent (e.g., ethanol). In another more specific aspect, the (17-β)-3-Oxoandrost-4-en-17-yl undecanoate comprises less than 2000 ppm ethanol. In another more specific aspect, the (17-β)-3-Oxoandrost-4-en-17-yl undecanoate comprises less than 1500 ppm of a solvent (e.g., ethanol). In a specific aspect, the (17-β)-3-Oxoandrost-4-en-17-yl undecanoate is a white crystalline or amorphous powder. In one specific aspect, the (17-β)-3-Oxoandrost-4-en-17-yl undecanoate composition comprises less than 3% or 2% of a single unknown impurity. In one specific aspect, the (17-β)-3-Oxoandrost-4-en-17-yl undecanoate composition comprises less than 1% of a single unknown impurity. In one specific aspect, the (17-β)-3-Oxoandrost-4-en-17-yl undecanoate composition comprises less than 0.5% of a single unknown impurity. In one specific aspect, the (17-β)-3-Oxoandrost-4-en-17-yl undecanoate composition comprises less than 0.1% of a single unknown impurity. In one specific aspect, the (17-β)-3-Oxoandrost-4-en-17-yl undecanoate composition comprises less than 5% of a single known impurity. In one specific aspect, the (17-β)-3-Oxoandrost-4-en-17-yl undecanoate composition comprises less than 3% of a single known impurity. In one specific aspect, the (17-β)-3-Oxoandrost-4-en-17-yl undecanoate composition comprises less than 2% of a single known impurity. In one specific aspect, the (17-β)-3-Oxoandrost-4-en-17-yl undecanoate composition comprises less than 1% of a single known impurity. In one specific aspect, the (17-β)-3-Oxoandrost-4-en-17-yl undecanoate composition comprises less than 0.5% of a single known impurity. In one aspect, the substantially pure (17-β)-3-Oxoandrost-4-en-17-yl undecanoate is substantially free of impurities specifically disclosed herein or in any of the Figures.

As used herein, an impurity is “known” if its structure or chemical identity is known and an impurity is “unknown” if its structure or chemical identity is known.

Thus, substantially pure (17-β)-3-Oxoandrost-4-en-17-yl undecanoate pharmaceutical ingredient or composition or dosage form comprising (17-β)-3-Oxoandrost-4-en-17-yl undecanoate which is useful for dosing of 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1900-2000, 2000-2500, 2500-3000, 3000-3500, 3500-4000, or 5000 or more mg per day to an individual, has greater than 80%, 85%, 90%, 95%, 98%, or 99% potency is provided. In one aspect, substantially pure (17-β)-3-Oxoandrost-4-en-17-yl undecanoate substantially free of impurities is provided. In another aspect, substantially pure (17-β)-3-Oxoandrost-4-en-17-yl undecanoate of having less than 20, 15, 10, 9, 8, 7, 6, 5, 4, 3, 2, or 1% of total impurities (known +unknown) is provided. In one aspect, substantially pure (17-β)-3-Oxoandrost-4-en-17-yl undecanoate having less than 10, 9, 8, 7, 6, 5, 4, 3, 2, or 1% of total unknown impurities is also provided. In one aspect, substantially pure (17-β)-3-Oxoandrost-4-en-17-yl undecanoate substantially free of any single known impurity is provided. In another aspect, substantially pure (17-β)-3-Oxoandrost-4-en-17-yl undecanoate having less than 10, 5, 2, 1, or 0.5% of any single known impurity is provided. In another aspect, substantially pure (17-β)-3-Oxoandrost-4-en-17-yl undecanoate substantially free of any single unknown impurity is provided. In one aspect, substantially pure (17-β)-3-Oxoandrost-4-en-17-yl undecanoate having less than 5, 4, 3, 2, 1, 0.5 or 0.2% or less of any single unknown impurity is provided. In one aspect, substantially pure (17-β)-3-Oxoandrost-4-en-17-yl undecanoate safe for administration to a human subject is provided (e.g., male or female). In one aspect, the substantial pure (17-β)-3-Oxoandrost-4-en-17-yl undecanoate is safe for chronic administration (e.g., more than 1, 2, 3, or 4 weeks; more than 1, 2, 3, 4, 6, 9, 12 months; more than 1, 2, 3, 4, or 5 years) at daily doses of 200 mg to 1500 mg (e.g., 250 to 1000 mg or 300 mg to 950 mg) to a human. In one aspect, the substantial pure (17-β)-3-Oxoandrost-4-en-17-yl undecanoate is safe for administration as a testosterone replacement therapy to a hypogonadal male. In one aspect, the substantially pure (17-β)-3-Oxoandrost-4-en-17-yl undecanoate is a stable. In one aspect, stable refers to having or maintaining a purity or impurity profile or characteristic described herein over time (e.g., 1, 2, 3, 4, 5, 6, 7, 8, 9, 10, 11, 12, 18 or 24 or more months). In one aspect, the substantially pure (17-β)-3-Oxoandrost-4-en-17-yl undecanoate having less than 20, 15, 10, 5 or 2% or less decomposition product of (17-β)-3-Oxoandrost-4-en-17-yl undecanoate is provided. In one aspect, the substantially pure (17-β)-3-Oxoandrost-4-en-17-yl undecanoate has less than 20, 15, 10, 5 or 2% or less decomposition product of (17-β)-3-Oxoandrost-4-en-17-yl undecanoate at 90 days at 20-25° C. is provided. In a related aspect, substantially pure (17-β)-3-Oxoandrost-4-en-17-yl undecanoate of the previous sentence wherein the decomposition product of (17-β)-3-Oxoandrost-4-en-17-yl undecanoate results from oxidation, reduction, cleavage of the ester moiety; oxidation of the steroid ring system; cleavage of one or more rings of the steroid ring system; rearrangement of the steroid ring; dealkylation of the steroid ring; dealkylation of the ester; or a combination thereof is provided. In one aspect, the substantially pure comprises more than 80, 85, 90, 95, 98, 99, 99.5 or 99.8% substantially (17-β)-3-Oxoandrost-4-en-17-yl undecanoate. In one aspect, the substantially pure (17-β)-3-Oxoandrost-4-en-17-yl undecanoate comprises less than 10, 5, 4, 3, 2, 1, or 0.5% 17-β-Hydroxy-4-Androsten-3-one. In one aspect, the substantially pure (17-β)-3-Oxoandrost-4-en-17-yl undecanoate comprising less than 50,000, 25000, or 15000 PPM of residual solvent (e.g., ethanol). In one aspect, a pharmaceutical composition comprising a pharmaceutically acceptable carrier and substantially pure (17-β)-3-Oxoandrost-4-en-17-yl undecanoate is provided. In one aspect, the pharmaceutical composition is stable or total impurities, total unknown impurities, any single known impurity, or any single unknown impurity does not increase above acceptable levels after 1 month, 3 months, 6 months, 9 months, one year, or two years at a specified temperature (e.g., 20, 25, 37, 40 or 60° C.) with no more than 10% or 20% decomposition or 10 or 20% loss in potency of (17-β)-3-Oxoandrost-4-en-17-yl undecanoate. In one aspect, the pharmaceutical composition comprises a stabilizing agent. In one aspect, the pharmaceutical composition comprises a pharmaceutically acceptable stabilizing agent which is an antioxidant, bufferant, complexing agent or desiccant. In one aspect, the pharmaceutical composition is formulated for topical, enteral or parenteral administration. In one aspect, the pharmaceutical composition is formulated for oral, buccal, sublingual, or sublabial administration. In one aspect, the pharmaceutical composition is formulated for nasal, rectal or vaginal administration. In one aspect, the pharmaceutical composition is formulated for intravenous, subcutaneous, intramuscular, intradermal, intraspinal, intrathecal, or intra-arterial administration. In one aspect, the pharmaceutical composition is a liquid, solution, suspension, dispersion, solid, semi-solid, a gel, a lotion, paste, foam, spray, emulsion, syrup, or ointment. In one aspect, the pharmaceutical composition is formulated as a tablet or capsule. In one aspect, the pharmaceutical composition is formulated as a tincture, patch, injectable, tablet, capsule, sprinkle, aggregate, granule, drink, or powder. In one aspect, a unit dosage form comprising a pharmaceutically acceptable carrier and the substantially pure (17-β)-3-Oxoandrost-4-en-17-yl undecanoate described herein which is useful for dosing up to 2000 mg per day to an individual, has greater than 80%, 85%, 90%, 95%, 98%, or 99% potency and a pharmaceutically acceptable carrier is provided. In one aspect, the unit dosage form comprises a stabilizing agent. In one aspect, the unit dosage form comprises a pharmaceutically acceptable stabilizing agent which is an antioxidant, bufferant, complexing agent or desiccant. In one aspect, the unit dosage form is formulated for topical, parenteral or enteral administration. In one aspect, the unit dosage form is a solid, a semi-solid, a gel, a lotion, a paste, tincture, foam, spray, suspension, dispersion, syrup, patch, or ointment. In one aspect, the unit dosage form is formulated for an oral route of administration. In one aspect, the unit dosage form is a tablet or capsule. In one aspect, the unit dosage form comprises or is prepared from at least 3, 5, 10, 15, 30, 25, 30, 40, 50, 60, 75, 100, 125, 150, 175, 200, 225, 250, 275 or 300 mg of substantially pure (17-β)-3-Oxoandrost-4-en-17-yl undecanoate. In one aspect, the substantially pure (17-β)-3-Oxoandrost-4-en-17-yl undecanoate is substantially free of impurities. In one aspect, the substantially pure (17-β)-3-Oxoandrost-4-en-17-yl undecanoate is substantially free of impurities specifically disclosed herein or in any of the Figures.

Synthetic Impurities

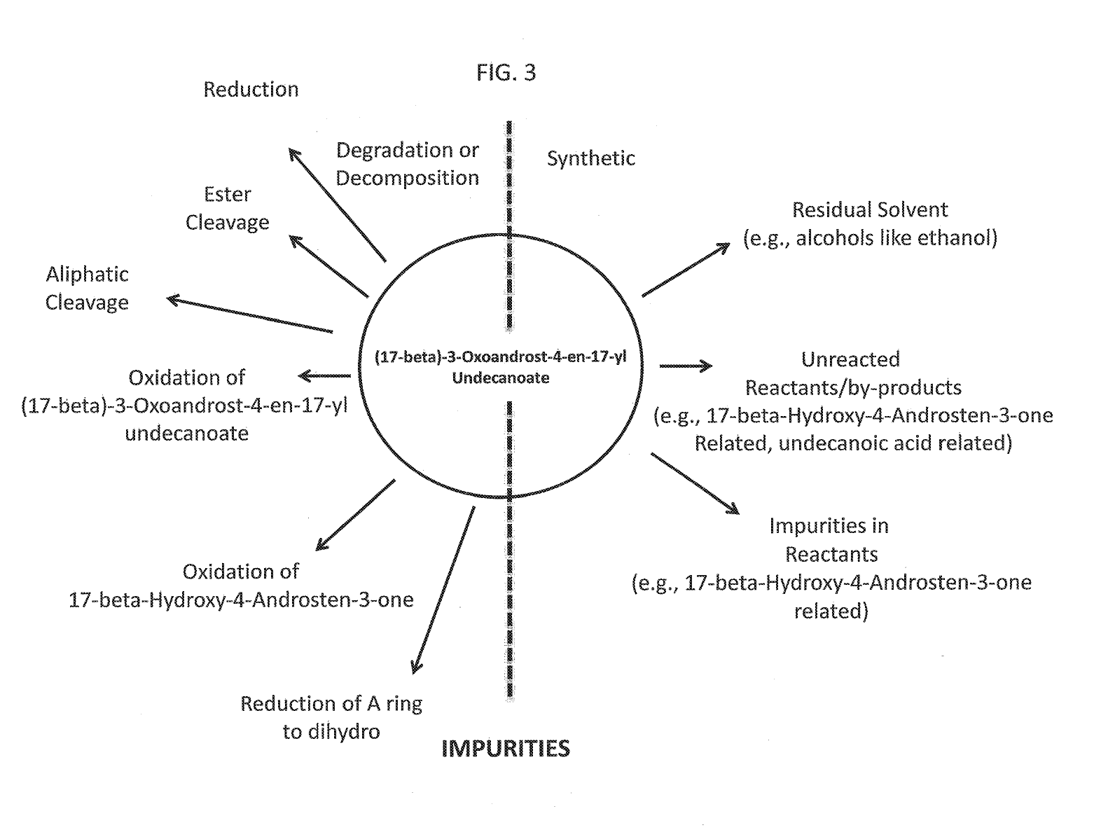

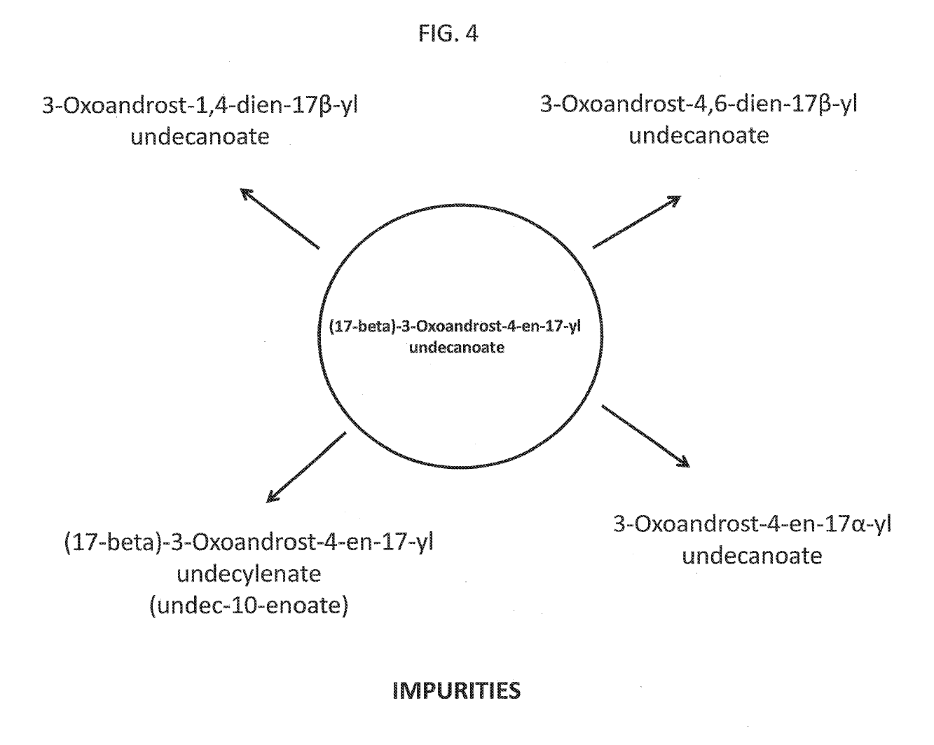

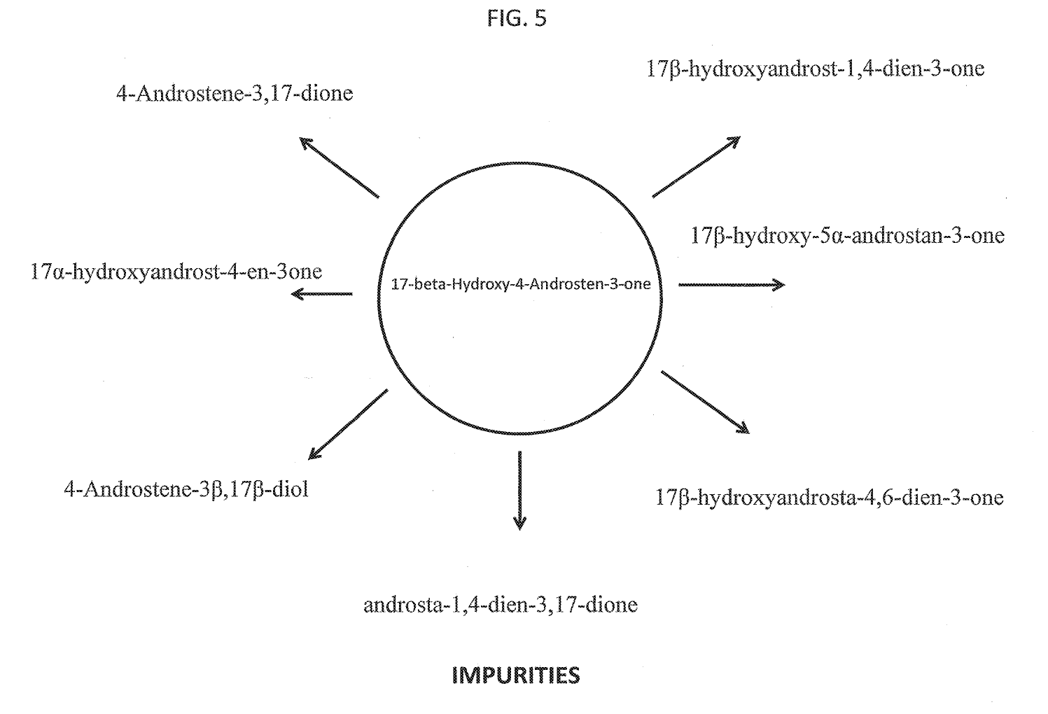

Provided herein are compositions containing (17-β)-3-Oxoandrost-4-en-17-yl undecanoate substantially free of synthetic impurities. Synthetic impurities of compositions containing (17-β)-3-Oxoandrost-4-en-17-yl undecanoate include, but are not limited to, residual solvents, unreactedto reactants, unreacted impurities in reactants, reaction products of impurities in reactants, and impuritieseated during synthesis, work up, or both, See FIG. 3.

In one embodiment, a composition having substantially pure (17-β)-3-Oxoandrost-4-en-17-yl undecanoate is provided. According to an aspect of this embodiment, the composition comprises at least 95% substantially pure (17-β)-3-Oxoandrost-4-en-17-yl undecanoate. In a more specific aspect, the composition comprises at least 97% substantially pure (17-β)-3-Oxoandrost-4-en-17-yl undecanoate. In another more specific aspect, the composition comprises at least 98% substantially pure (17-β)-3-Oxoandrost-4-en-17-yl undecanoate. In another more specific aspect, the composition comprises at least 99% substantially (17-β)-3-Oxoandrost-4-en-17-yl undecanoate. In another more specific aspect, the composition comprises at least 99.5% substantially pure (17-β)-3-Oxoandrost-4-en-17-yl undecanoate. In a specific aspect, the API is a crystalline or amorphous powder. in one aspect, a substantially pure (17-β)-3-Oxoandrost-4-en-17-yl undecanoate substantially free of synthetic impurity (A): 17-β-Hydroxy-4-Androsten-3-one or a synthetic reaction by-product thereof is provided. In one aspect, a substantially pure (17-β)-3-Oxoandrost-4-en-17-yl undecanoate substantially free of synthetic impurity (B): undecanoic acid, undecanoate, undecanoyl chloride or a non-API synthetic reaction by-product thereof is provided. In one aspect, a substantially pure (17-β)-3-Oxoandrost-4-en-17-yl undecanoate substantially free of synthetic impurity (C): Ethanol, pyridine, heptanes, heptanes, or a combination thereof is provided. In one aspect, a substantially pure (17-β)-3-Oxoandrost-4-en-17-yl undecanoate substantially free of synthetic impurity (D): (17-β)-3-Oxoandrost-4-en-17-yl undecanoate is provided. In one aspect, a substantially pure (17-β)-3-Oxoandrost-4-en-17-yl undecanoate substantially free of synthetic impurity (E): (17-β)-3-Oxoandrost-4-en-17-yl decanoate is provided. In one aspect, a substantially pure (17-β)-3-Oxoandrost-4-en-17-yl undecanoate substantially free of synthetic impurity (F): (17-β)-3-Oxoandrost-4-en-17-yl dodecanoate is provided. In one aspect, a substantially pure (17-β)-3-Oxoandrost-4-en-17-yl undecanoate substantially free of synthetic impurity (E): (17-β)-3-Oxoandrost-4-en-17-yl tetradecanoate is provided. According to some aspect, substantially free of an impurity in this paragraph refers to less than 10%, 7%, 5%, 3%, 3%, 1%, 0.5%, 0.1%, 0.05%, or 0.01% of any specific impurity referred to this paragraph or their combined total. In a specific aspect, the sum total of all these Impurities (Synthetic Impurity A, B, C, I3, or E) compared to the amount of (17-β)-3-Oxoandrost-4-en-17-yl undecanoate in a sample as determined by HPLC (e.g., at about 240 nm or another appropriate wavelength) is no more than 20%, 15%, 10%, 5%, 4%, 3%, 2%, 1%, 0.75%, 0.50%, 0,25%, 0.20%, 0.10%, or 0.50%. In another specific aspect, the total of any one of these synthetic impurity A, B, C, or E as described above as compared to the amount of (17-β)-3-Oxoandrost-4-en-17-yl undecanoate in a sample as determined by HPLC (e.g., at about 240 nm or 242 nrn another appropriate wavelength) is no more than 2%, 1.5%, 1.0%, 0.50%, 0.40%, 0.30%, 0.20%, 0.10%, 0.075%, 0.050%, 0.025%, 0.020%, 0.010%, or 0.0050%.

Decomposition or Degradation (17-β)-3-Oxoandrost-4-en-17-ylUndecanoate or Incompatibility Thereof with Carrier of the Composition or Related Impurities

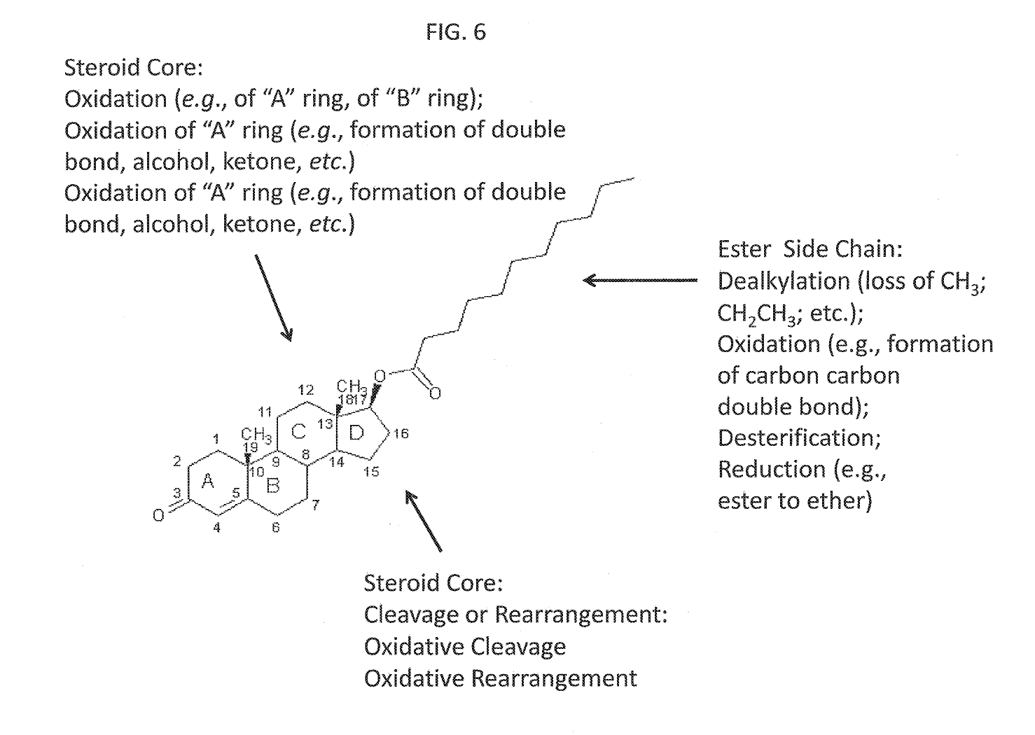

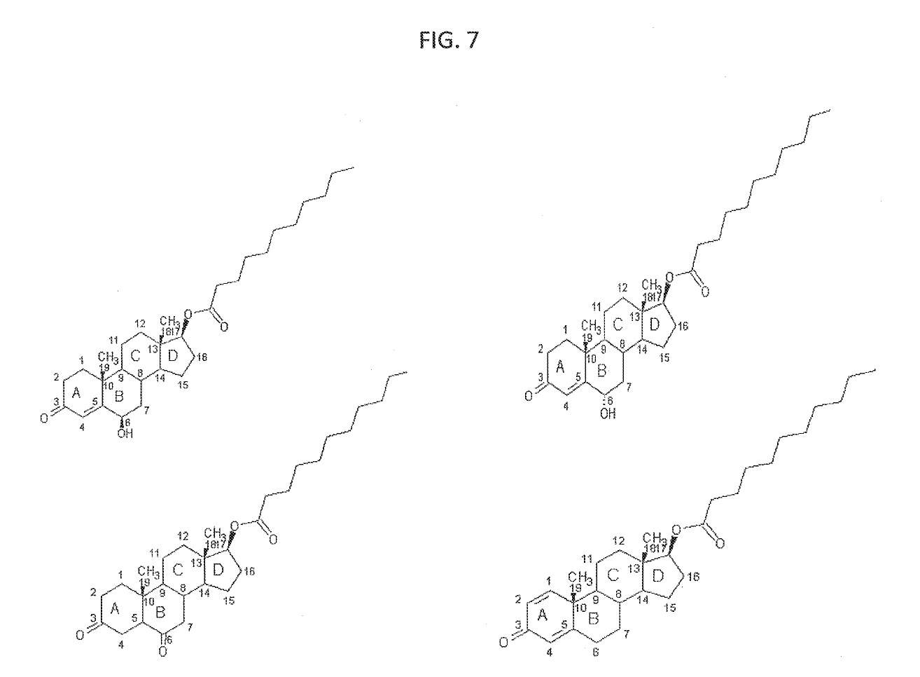

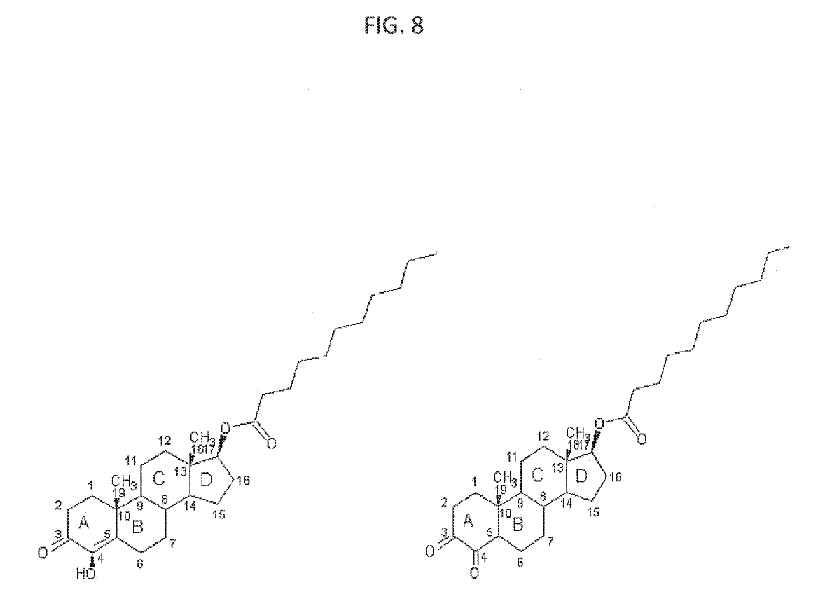

Provided herein are compositions containing substantially pure (17-β)-3-Oxoandrost-4-en-17-yl undecanoate substantially free of decomposition impurities. Decomposition impurities of compositions containing (17-β)-3-Oxoandrost-4-en-17-yl undecanoate include, but are not limited to, decomposition of (17-β)-3-Oxoandrost-4-en-17-yl undecanoate in e.g., bulk (17-β)-3-Oxoandrost-4-en-17-yl undecanoate, pharmaceutical compositions or formulations, or in unit dosage forms. Decomposition impurities include, but are not limited to impurities resulting from cleavage of the ester moiety, oxidation of functional groups on the steroid polycyclic core, dealkylation of the ester, etc. In some aspects, stabilized compositions are provided herein. Such stabilized compositions can be e.g., compositions stored under specific environmental conditions, have stabilizing agents (e.g., an antioxidant), or both. The stabilized composition can be (17-β)-3-Oxoandrost-4-en-17-yl undecanoate, a composition such as a pharmaceutical composition comprising (17-β)-3-Oxoandrost-4-en-17-yl undecanoate and a pharmaceutically acceptable carrier, a unit dosage form having (17-β)-3-Oxoandrost-4-en-17-yl undecanoate and a pharmaceutically acceptable carrier and the such. In one embodiment, substantially pure (17-β)-3-Oxoandrost-4-en-17-yl undecanoate is provided substantially free of Decomposition Impurity (1): (17-β)-Hydroxy-4-Androsten-3-one. In one aspect, substantially pure (17-β)-3-Oxoandrost-4-en-17-yl undecanoate is provided that is substantially free of Decomposition Impurity (2): hydroxylated at position 6 (out of the plane) of the FIG. 6 (one of top structures of FIG. 7 also). In one aspect, substantially pure (17-β)-3-Oxoandrost-4-en-17-yl undecanoate is provided that is substantially free of Decomposition Impurity (3): hydroxylated at position 6 (in the plane) of the FIG. 6 (one of top structures of FIG. 7 also). In one aspect, a substantially pure (17-β)-3-Oxoandrost-4-en-17-yl undecanoate substantially free of Decomposition Impurity (1), (2), or (3) or a combination thereof as described above is provided. In a specific aspect, the sum total of all these Decomposition Impurities (Decomposition Impurity 1, 2, and 3) compared to the amount of (17-β)-3-Oxoandrost-4-en-17-yl undecanoate in a sample as determined by HPLC (e.g., at about 240 or 242 nm or another appropriate wavelength) is no more than 20%, 15%, 10%, 5%, 4%, 3%, 2%, 1%, 0.75%, 0.50%, 0.25%, 0.20%, 0.10%, or 0.50%. In another specific aspect, the total of any one of Decomposition Impurities (1), (2), and (3) described above as compared to the amount of (17-β)-3-Oxoandrost-4-en-17-yl undecanoate in a sample as determined by HPLC (e.g., at about 240 or 242 nm or another appropriate wavelength) is no more than 2%, 1.5%, 1.0%, 0.50%, 0.40%, 0.30%, 0.20%, 0.10%, 0.075%, 0,050%, 0.025%, 0.020%, 0.010%, or 0.0050%. In a specific aspect, the sum total of all impurities compared to the amount of (17-β)-3-Oxoandrost-4-en-17-yl undecanoate in a sample as determined by HPLC (e.g., at about 240 or 242 nm or another appropriate wavelength) is no more than 20%, 15%, 10%, 5%, 4%, 3%, 2%, 1%, 0.75%, 0.50%, 0.25%, 0.20%, 0.10%, or 0.50%. In another specific aspect, the total of any one of these impurities as identified in the Figures compared to the amount of (17-β)-3-Oxoandrost-4-en-17-yl undecanoate in a sample as determined by HPLC (e.g., at about 240 nm or another appropriate wavelength) is no more than 2%, 1.5%, 1.0%, 0.50%, 0.40%, 0.30%, 0.20%, 0.10%, 0.075%, 0,050%, 0.025%, 0.020%, 0.010%, or 0.0050%. It was found that inclusion of a stabilizing agent in these formulations like ascorbyl palmitate prevents or substantially inhibits the formation of impurities (e.g., decomposition impurity (2) and (3) and others) for samples stored over amounts of time e.g., 1 month, 3 months, 6 months, 12 months, 2 years.

Determination of Impurities and Potency

Impurities in the composition can be determined by any method suitable for identifying such impurities. Typical methods for determining impurities depend on the nature of the starting composition. For example, techniques suitable for examining bulk (17-β)-3-Oxoandrost-4-en-17-yl undecanoate may be different or involve different condition as compared to examining impurities in a pharmaceutical composition or unit dosage form. The potency of bulk API, pharmaceutical compositions or unit dosage forms may also be determined via some of these techniques. Exemplary, non-limiting techniques are described in more detail below.

High Performance Liquid Chromatography (HPLC)

HPLC is a technique commonly used to identify impurities or levels thereof in substances (and may also be used to determine potency). HPLC can be used to quantitatively or to qualitatively assess impurities in samples (e.g., bulk (17-β)-3-Oxoandrost-4-en-17-yl undecanoate or a composition containing (17-β)-3-Oxoandrost-4-en-17-yl undecanoate) and potency. HPLC methods can also be used for assessing the potency of a particular sample. The ordinary skilled artisan is familiar with and capable of performing HPLC techniques. Typically, a chromatogram from known standard samples is compared to that of an unknown sample. The purity of the unknown can be estimated by comparing the area under the curve for the peak that corresponds to (17-β)-3-Oxoandrost-4-en-17-yl undecanoate to other peaks in the chromatogram. Several appropriate HPLC techniques are described in the Examples. Other methods can be adapted to analyze purity or impurities of the compositions described herein (See e.g., Gonzalo-Lumbreras, et al. J. Pharm. Biomed. Anal. Volume 38, Issue 4, 15 July 2005, Pages 757-762; Pozo et al. Biomed. Chrofn. Volume 23, Issue 8, pages 873-880, August 2009).

Nuclear Magnetic Resonance (NMR)

NMR is a technique commonly used to identify impurities or levels thereof in substances and potency. NMR can be used to quantitatively or to qualitatively assess impurities in samples (e.g., bulk (17-β)-3-Oxoandrost-4-en-17-yl undecanoate or a composition containing (17-β)-3-Oxoandrost-4-en-17-yl undecanoate) and potency. NMR methods can also be used for assessing the potency of a particular sample. The ordinary skilled artisan is familiar with and capable of performing NMR techniques. Typically, proton NMR, ¹³C NMR, or both are used for assessing (17-β)-3-Oxoandrost-4-en-17-yl undecanoate compositions for purity or potency (quantitative NMR). Webster et al. Appl Spectrosc. 2010 May;64(5):537-42.

Other techniques are also useful for analyzing (17-β)-3-Oxoandrost-4-en-17-yl undecanoate for purity and potency such as x-ray powder diffraction, mass spectrometry, Fourier-transform infrared spectroscopy, Raman spectroscopy, etc. See Bugay Adv Drug Deliv Rev. 2001 May 16;48(1):43-65.

Stabilized Compositions

Stabilized compositions are provided herein. Instability of bulk (17-β)-3-Oxoandrost-4-en-17-yl undecanoate and composition containing (17-β)-3-Oxoandrost-4-en-17-yl undecanoate can result in undesirable changes in performance (e.g., dissolution or bioavailability), changes in physical appearance, product failures, safety, toxicity, etc. Stabilization of compositions having (17-β)-3-Oxoandrost-4-en-17-yl undecanoate depends on the nature of the composition and the nature of what type of stabilization is desired. For example, stabilization of bulk (17-β)-3-Oxoandrost-4-en-17-yl undecanoate may involve a specific set of conditions (“stabilizing condition”) e.g., storage and handling conditions that are different (although not necessarily) than for a pharmaceutical composition or a unit dosage form. Furthermore, stabilization of certain compositions can involve preventing or mitigating certain things in one composition whereas those same certain things may be desirable in another composition. For example, crystalline substantially pure and substantially free of impurity (17-β)-3-Oxoandrost-4-en-17-yl undecanoate may be desirable for stabilization of (17-β)-3-Oxoandrost-4-en-17-yl undecanoate, whereas in other compositions like a pharmaceutical compositions or unit dosage forms crystalline (17-β)-3-Oxoandrost-4-en-17-yl undecanoate may not he desirable.

Thus, in one embodiment, substantially pure (17-β)-3-Oxoandrost-4-en-17-yl undecanoate is provided that is stabilized. In one aspect of this embodiment, the stabilized substantially pure (17-β)-3-Oxoandrost-4-en-17-yl undecanoate is stabilized by its high degree of purity in solid fort (e.g., crystalline, amorphous, or a combination thereof), In one aspect, the (17-β)-3-Oxoandrost-4-en-17-yl undecanoate is recrystallized from a solvent. In one aspect, the (17-β)-3-Oxoandrost-4-en-17-yl undecanoate is recrystallized from a solvent which is an alcohol, alkane, oil, fatty acid or other solvent. In one aspect, the (17-β)-3-Oxoandrost-4-en-17-yl undecanoate is recrystallized from a solvent which is an alcohol, alkane, oil, fatty acid or other solvent to provide substantially pure stabilized (17-β)-3-Oxoandrost-4-en-17-yl undecanoate pharmaceutical ingredient. In another aspect, the substantially pure (17-β)-3-Oxoandrost-4-en-17-yl undecanoate is stabilized in a composition e.g., pharmaceutical composition or unit dosage form that has a stabilizing agent. In a specific aspect, the stabilizing agent is an antioxidant, In another specific aspect, the stabilizing agent is ascorbate or a derivative thereof In another specific aspect, the stabilizing agent is a fatty acid ester of ascorbate. In another specific aspect, the stabilizing agent is ascorbyl palmitate. in some aspects, compositions are provided (e.g., pharmaceutical composition, formulation or unit dosage form) having or prepared from substantially pure (17-β)-3-Oxoandrost-4-en-17-yl undecanoate and ascorbyl palmitate. In one aspect of this embodiment, the stabilized substantial pure (17-β)-3-Oxoandrost-4-en-17-yl undecanoate is substantially free of impurities.

Bulk API can also be stabilized by maintaining the substantially pure (17-β)-3-Oxoandrost-4-en-17-yl undecanoate under stabilizing conditions which include, but are not limited to, temperature (e.g., less than 0, 5, 10, 15, 20, 25, 30, 35, 40, 45, 50, 55, 60° C). ; relative humidity (e.g., less than 90, 80, 70, 60, 50, 40, 30, 20, 10 or 5%); light (e.g., controlling or minimizing exposure to light including UV, visible, or IR light); and oxidation (e.g., preventing or minimizing exposure to oxidizing agents or oxidizing conditions). In one aspect, bulk substantially pure (17-β)-3-Oxoandrost-4-en-17-yl undecanoate in an amount of greater than 1 g, 5 g, 10 g, 50 g, 100 g, 500 g, 1 kg, 5 kg, 10 kg, 50 kg, 100 kg, 500 kg, 1000 kg, 5000 kg, or 10,000 kg is maintained under stabilizing conditions. In an aspect, the substantially pure (17-β)-3-Oxoandrost-4-en-17-yl undecanoate is crystalline API. In a related aspect, methods of stabilizing (17-β)-3-Oxoandrost-4-en-17-yl undecanoate are also provided herein. The method can depend on the composition (e.g., bulk substantially pure (17-β)-3-Oxoandrost-4-en-17-yl undecanoate, pharmaceutical composition, formulation, or unit dosage form) that is to be stabilized. In one aspect, the method comprises storing substantially pure (17-β)-3-Oxoandrost-4-en-17-yl undecanoate under conditions that prevent decomposition of the (17-β)-3-Oxoandrost-4-en-17-yl undecanoate. According to a specific aspect, the condition is high purity (e.g., greater than 95%, greater than 98% or greater than 99% by total weight of the composition), maintaining the composition at a temperature of less than 60° C., less than 50° C., less than 40° C., less than 30° C., less than 20° C., less than 15° C., less than 10° C., less than 5° C., or less than 0° C., maintaining the composition in a non-oxidizing environment or minimal oxidizing environment. In yet another aspect, the method of stabilizing comprises combining (17-β)-3-Oxoandrost-4-en-17-yl undecanoate with a carrier or agent (e.g., fatty acid ester of ascorbate (i.e., ascorbyl palmitate)) that prevents or minimizes decomposition of (17-β)-3-Oxoandrost-4-en-17-yl undecanoate. These methods are suitable for providing substantially pure (17-β)-3-Oxoandrost-4-en-17-yl undecanoate or a unit dosage form or pharmaceutical composition prepared from or comprising (17-β)-3-Oxoandrost-4-en-17-yl undecanoate wherein there is less than 5%, 2%, or 1% total API related impurities after storage for a period of time of 1, 2, 3, 4, 5, 6, 7, 8, 9, 10, 12, 15, 17, 20, 25, or 30 months or longer. According to some aspects, the composition has a potency of 100% or 99.5% or more, 99% or more, 98% or more, 97% or more, 95% or more, or 90% or 80% or more after storage for a period of time. According to some aspects, the composition has a purity of 100% or 99.5% or more, 99% or more, 98% or more, 97% or more, 95% or more, or 90% or more after storage for a period of time.

In certain embodiments, the pharmaceutical compositions are substantial free of peroxides. For example, in some embodiments the pharmaceutical composition or components from which it is prepared have a peroxide of less than 100, 90, 80, 70, 60, 50, 40, 30, 25, 20, 15, 10 or 5.

Oral Pharmaceutical Compositions Having Substantially Pure (17-β)-3-Oxoandrost-4-en-17-yl Undecanoate

The pharmaceutical compositions and dosage forms (e.g. capsule or tablet) described herein prepared from or comprising substantially pure (17-β)-3-Oxoandrost-4-en-17-yl undecanoate (as described in the embodiments above) can include a variety of pharmaceutically acceptable carriers or additives known in the art. Non-limited examples of components that can be included as components of the pharmaceutical carrier include lipophilic surfactants, hydrophilic surfactants, triglycerides, fatty acid (C8 to C22), fatty acid glycerides (mono-, di-, tri-, or a combination thereof), or a combination thereof.

In one embodiment, the pharmaceutical composition or dosage form comprises, or is prepared with, substantially pure (17-β)-3-Oxoandrost-4-en-17-yl undecanoate as described herein. In one embodiment, the pharmaceutical composition or unit dosage form is characterized by its dissolution or release profile (e.g., at least 75% in 4 hours). In one aspect, the oral pharmaceutical composition or unit dosage form can be formulated as a tablet or capsule (e.g., hard gel or soft gel). According to this embodiment, a pharmaceutical composition or unit dosage form having a particular amount of substantially pure (17-β)-3-Oxoandrost-4-en-17-yl undecanoate (e.g., 3 mg or more, 4 mg or more, 5 mg or more, 10 mg or more, 15 mg or more, 20 mg or more, 30 mg or more, 40 mg or more, 50 mg or more, 75 mg or more, 100 mg or more, 125 mg or more, 150 mg or more, 175 mg or more, 200 mg or more, 225 mg or more, 250 mg or more, 275 mg or more, or 300 mg or more).

In one embodiment, the pharmaceutical composition or unit dosage form having (or prepared from) substantially pure (17-β)-3-Oxoandrost-4-en-17-yl undecanoate has a release profile (e.g., a profile comprising 2, 3, 4, 5, or 6 or more time points each at least 5, 10, or 15 minutes apart or a single time point) of (17-β)-3-Oxoandrost-4-en-17-yl undecanoate that does not change substantially as a function of storage time using a USP type 2 apparatus in about 1000 mL 8% Triton X100 solution in water at a specific temperature (e.g., 20.0, 37.0 or 40.0° C. (±0.5) at 100 rpm. In one aspect, the release profile does not substantially change over a period of 1, 2, 3, 4, 5, 6, 7, 8, 9, 10, 11, 12, 13, 14, 15, or 16 weeks. In one aspect, the release profile does not substantially change over a period of 1, 2, 3, 4, 5, 6, 7, 8, 9, 10, 11, 12, 13, 14, 15, or 16 months.

In one embodiment, the pharmaceutical composition or unit dosage form having (or prepared from) substantially pure (17-β)-3-Oxoandrost-4-en-17-yl undecanoate has a release profile (e.g., a profile comprising 2, 3, 4, 5, or 6 or more time points each at least 5, 10, or 15 minutes apart or a single time point) of (17-β)-3-Oxoandrost-4-en-17-yl undecanoate using a USP type 2 apparatus in about 1000 mL 8% Triton X100 solution in water at a specific temperature (e.g., 20.0, 37.0 or 40.0° C. (±0.5) at 100 rpm that release at least 10, 20, 30, 40, 50, 60, 70, 75, 80, 85, 90, 95, 96, 97, 98, or 99% at 15, 20, 30, 40, 45, 50, 60, 90, 120, 180, 240, or 300 minutes.

In one embodiment, the pharmaceutical composition or unit dosage form having (or prepared from) substantially pure (17-β)-3-Oxoandrost-4-en-17-yl undecanoate has a release profile (e.g., a profile comprising 2, 3, 4, 5, or 6 or more time points each at least 5, 10, or 15 minutes apart or a single time point) of (17-β)-3-Oxoandrost-4-en-17-yl undecanoate using a USP type 2 apparatus in about 1000 mL 8% Triton X100 solution in water at a specific temperature (e.g., 20.0, 37.0 or 40.0° C. (±0.5) at 100 rpm that release less than 10, 20, 30, 40, 50, 60, 70, 75, 80, 85, 90, 95, 96, 97, 98, or 99% at 15, 20, 30, 40, 45, 50, 60, 90, 120, 180, 240, or 300 minutes.

In some embodiments, the pharmaceutically acceptable carrier of the composition (e.g., pharmaceutical composition, formulation or unit dosage form) can include a lipophilic additive.

In some embodiments, the lipophilic additive can comprise at least about 10, 20, 30, 40, 50, 60, 70, 80, 90 or 95 wt % of the pharmaceutically acceptable carrier. Non-limiting examples of lipophilic additives can include lipophilic surfactants, triglycerides, tocopherol, tocopherol derivatives and combinations thereof. In one embodiment, the lipophilic additive can include a fatty acid or fatty acid glyceride. In another embodiment, lipophilic additive can include the fatty acid glyceride, and the fatty acid glyceride can be a monoglyceride, a diglyceride, or mixtures thereof. In one aspect, the fatty acid is oleic acid, stearic acid or a combination thereof. In one aspect, the fatty acid glyceride is glyceryl palmitostearate. Non-limiting examples of fatty acid glycerides that can be used in the oral pharmaceutical compositions and dosage forms of the present invention include monoglycerides and/or diglycerides derived from sources such as maize oil, poppy seed oil, safflower oil, sunflower oil, borage seed oil, peppermint oil, coconut oil, palm kernel oil, castor oil, and mixtures thereof. In one embodiment, the pharmaceutical composition or dosage form thereof comprises 50%, 40%, 30%, 20%, 15%, 10%, 5% by weight or less of a triglyceride. In a specific embodiment, the pharmaceutical composition or dosage form thereof, comprises less than 50% by weight of castor oil. In another embodiment, the composition includes 10 wt % or less of triglycerides. In a further embodiment, the composition includes 5 wt % or less of triglycerides. In a still a further embodiment, the composition includes about 3 wt % or less of triglycerides. In still a further embodiment, the composition includes about 1 wt % or less of triglycerides. In another embodiment, the composition is free or substantially free of triglycerides. In another embodiment, the composition and dosage forms are free of phytosterols and phytosterol fatty acid esters.

In another embodiment, the lipophilic additive can include a lipophilic surfactant.

As used herein a surfactant is considered to be a lipophilic surfactant when it has an HLB value of 10 or less. Various lipophilic surfactants can be used including, but not limited to mono-, di-glycerides of fatty acids like glyceryl monolinoleate (e.g. Maisine® 35-1), mono- and di glycerides of caprylic, capric acid (e.g. Capmul® MCM), glyceryl monooleate, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil® M 2125 CS), PEG-6 almond oil (e.g. Labrafil®M 1966 CS), PEG-6 apricot kernel oil (e.g. Labrafil®M 1944 CS), PEG-6 olive oil (e.g.Labrafil®M 1980 CS), PEG-6 peanut oil (e.g. Labrafil®M 1969 CS), PEG-6 hydrogenated palm kernel oil (e.g. Labrafil®. M 2130 BS), PEG-6 palm kernel oil (e.g. Labrafil® M 2130 CS), PEG-6 triolein (e.g. Labrafil® M 2735 CS), PEG-8 corn oil (e.g. Labrafil® WL 2609 BS), PEG-20 corn glycerides (e.g. Crovol® M40), PEG-20 almond glycerides (e.g. Crovol® A40), lipophilic polyoxyethylene-polyoxypropylene block co-polymers (e.g. Pluronic® L92, L101, L121 etc.); propylene glycol fatty acid esters, such as propylene glycol monolaurate (e.g. Lauroglycol FCC), propylene glycol ricinoleate (e.g. Propymuls), propylene glycol monooleate (e.g. Myverol P-06), propylene glycol dicaprylate/dicaprate (e.g. Captex® 200), and propylene glycol dioctanoate (e.g. Captex® 800), propylene glycol mono-caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate ; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. It is important to note that some lipophilic surfactants may also function as the solubilizer component of the compositions and oral dosage forms.

In one embodiment, the lipophilic surfactant can be selected from the group consisting of glyceryl monolinoleate (e.g. Maisine® 35-1), mono- and di glycerides of caprylic, capric acid (e.g. Capmul® MCM), glyceryl monooleate, propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (e.g. Arlacel 20), sorbitan monopalmitate, sorbitan monooleate , sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof. In some embodiments, the lipophilic surfactants can comprise at least about 10, 20, 30, 40, 50, 60, 70, 80, 90 or 95 wt % of the total pharmaceutically acceptable carrier. It should be noted that the combinations of two or more lipophilic surfactants from the same or different classes therein are also within the scope of this invention and are together can be referred to as the lipophilic surfactant, unless otherwise stated.

In some embodiments of the present invention, the oral pharmaceutical compositions or dosage forms (e.g. capsule or tablet) can include a hydrophilic additive. In one embodiment, hydrophilic additive is a selected from the group consisting of hydrophilic surfactant, celluloses—such as hydroxypropyl celluloses low molecular weight, low viscosity types (e.g. Methocel® E5, E6, E15, LV100 etc. grades) and hydroxypropyl celluloses having higher molecular weight, medium to high viscosity (e.g. Methocel® K4M, K15M, K100M etc); polyvinylpyrrolidones (e.g. Kollidon k17, K30 etc); polyvinyl acetates and combinations thereof

In one embodiment, the hydrophilic additive can be a hydrophilic surfactant. A surfactant is considered to be a hydrophilic surfactant when it has an HLB value of greater than 10. Non-limiting examples of hydrophilic surfactants include non-ionic surfactants, ionic surfactants and zwitterionic surfactants. Specifically the hydrophilic surfactants suitable for the current invention include, but not limited to alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono-and di-ester mixtures; polysorbates, polyethylene glycol derivatives of tocopherol and the like It should be noted that the combinations of two or more hydrophilic surfactants from the same or different classes are within the scope of this invention and are together can be referred to as the hydrophilic surfactant unless explicitly specified. In one embodiment, the hydrophilic additive can be a hydrophilic surfactant. Non-limiting examples of hydrophilic surfactants can include PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, and mixtures thereof

In some embodiments, surfactants utilized in the pharmaceutical compositions described herein include sterols and derivatives of sterols. In various embodiments, these surfactants are hydrophilic or lipophilic. Examples of hydrophilic sterol surfactants are lanosterol PEG-24 cholesterol ether (e.g. Solulan C-24, Amerchol), PEG-30 soya sterol (e.g. Nikkol BPS-30, from Nikko), PEG-25 phytosterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko).

In one embodiment, the oral pharmaceutical composition or the dosage form comprises or is prepared from substantially pure (17-β)-3-Oxoandrost-4-en-17-yl undecanoate and a pharmaceutically acceptable carrier, wherein substantially pure (17-β)-3-Oxoandrost-4-en-17-yl undecanoate comprises about 0.5 wt % to about 50 wt %, 5 wt % to about 45 wt %, 15 wt % to about 40 wt %, 25 wt % to about 35 wt %, or 26 wt % to about 32 wt % of the composition or dosage form. In another embodiment, the compositions or the dosage form of the current invention includes substantially pure (17-β)-3-Oxoandrost-4-en-17-yl undecanoate and a pharmaceutically acceptable carrier, wherein substantially pure (17-β)-3-Oxoandrost-4-en-17-yl undecanoate comprises about 0.5 wt % to about 50 wt %, 5 wt % to about 45 wt %, 15 wt % to about 40 wt %, 25 wt % to about 35 wt %, or 26 wt % to about 32 wt % of the composition or dosage form, and wherein the carrier includes at least 50 wt % of the composition or the dosage form and wherein the substantially pure (17-β)-3-Oxoandrost-4-en-17-yl undecanoate is not solubilized at 30° C., or above 30° C., or at a temperature range above 30° C., including 30° C. to about 40° C. In an additional more specific embodiment, the substantially pure (17-β)-3-Oxoandrost-4-en-17-yl undecanoate is not fully dissolved in the carrier at human body temperature. In one aspect, the substantially pure (17-β)-3-Oxoandrost-4-en-17-yl undecanoate is substantially free of impurities. In one aspect, the pharmaceutical composition or unit dosage form is formulated as a tablet or capsule (e.g., soft gel or hard gel). In one aspect, the pharmaceutical composition or unit dosage form further comprises one or more additives (e.g., 2, 3, 4, 5, 6, 7, 8, 9, or 10). In one aspect, the pharmaceutical composition or unit dosage form comprises a particular amount of substantially pure (17-β)-3-Oxoandrost-4-en-17-yl undecanoate (e.g., 3 mg or more, 4 mg or more, 5 mg or more, 10 mg or more, 15 mg or more, 20 mg or more, 30 mg or more, 40 mg or more, 50 mg or more, 75 mg or more, 100 mg or more, 125 mg or more, 150 mg or more, 175 mg or more, 200 mg or more, 225 mg or more, 250 mg or more, 275 mg or more, or 300 mg or more) and typically less than 600 mg. In one aspect, the substantially pure (17-β)-3-Oxoandrost-4-en-17-yl undecanoate is substantially free of impurities specifically disclosed herein or in any of the Figures.

In another embodiment, the compositions or the dosage forms includes or is prepared from substantially pure (17-β)-3-Oxoandrost-4-en-17-yl undecanoate and a pharmaceutically acceptable carrier, wherein the substantially pure (17-β)-3-Oxoandrost-4-en-17-yl undecanoate comprises about 0.5 wt % to about 50 wt %, 5 wt % to about 45 wt %, 15 wt % to about 40 wt %, 25 wt % to about 35 wt %, or 26 wt % to about 32 wt % of the composition or the dosage form, and wherein the carrier includes about 50 wt % to about100 wt % of lipophilic surfactant and 0 wt % to about 50 wt % of hydrophilic surfactant. In a further embodiment, the substantially pure (17-β)-3-Oxoandrost-4-en-17-yl undecanoate is not solubilized at 30° C., or above 30° C., or at a temperature range above 30° C., including 30° C. to about 40° C. In an additional more specific embodiment, the substantially pure (17-β)-3-Oxoandrost-4-en-17-yl undecanoate is not fully dissolved in the carrier at human body temperature. In one aspect, the pharmaceutical composition or unit dosage form is formulated as a tablet or capsule (e.g., soft gel or hard gel). In one aspect, the pharmaceutical composition or unit dosage form further comprises one or more additives (e.g., 2, 3, 4, 5, 6, 7, 8, 9, or 10). In one aspect, the pharmaceutical composition or unit dosage form comprises a particular amount of substantially pure (17-β)-3-Oxoandrost-4-en-17-yl undecanoate (e.g., 3 mg or more, 4 mg or more, 5 mg or more, 10 mg or more, 15 mg or more, 20 mg or more, 30 mg or more, 40 mg or more, 50 mg or more, 75 mg or more, 100 mg or more, 125 mg or more, 150 mg or more, 175 mg or more, 200 mg or more, 225 mg or more, 250 mg or more, 275 mg or more, or 300 mg or more) and typically less than 600 mg. In one aspect, the substantially pure (17-β)-3-Oxoandrost-4-en-17-yl undecanoate is substantially free of impurities specifically disclosed herein or in any of the Figures.

In another specific embodiment, the composition or the dosage form includes or is prepared from substantially pure (17-β)-3-Oxoandrost-4-en-l7-yl undecanoate and a pharmaceutically acceptable carrier, wherein substantially pure (17-β)-3-Oxoandrost-4-en-17-yl undecanoate comprises about 0.5 wt % to about 50 wt %, 5 wt % to about 45 wt %, 15 wt % to about 40 wt %, 25 wt % to about 35 wt %, or 26 wt % to about 32 wt % of the composition or the dosage form, and the carrier includes about 50 wt % to about 95 wt % a lipophilic surfactant and a hydrophilic surfactant 5 wt % to about 30 wt %. In one aspect, the lipophilic additive is a C16 to C18 fatty acid (saturated or having 1, 2, or 3 unsaturations), or a mono-, di-, or triglyceride thereof. In one aspect, mono-, di-, or tri glyceride is glyceryl palmitostearate. In one aspect, the hydrophilic component is a hydrogenated oil. In one aspect, the hydrophilic component is a polyoxylated hydrogenated oil. In a further more specific embodiment, the substantially pure (17-β)-3-Oxoandrost-4-en-17-yl undecanoate is not solubilized at 30° C., or above 30° C., or at a temperature range above 30° C., including 30° C. to about 40° C. In an additional more specific embodiment, the ester is not fully dissolved in the carrier at human body temperature. In another more specific embodiment, the composition or the dosage form can optionally contain about 10 wt % or less of ethyl alcohol. In an additional more specific embodiment, the substantially pure (17-β)-3-Oxoandrost-4-en-17-yl undecanoate is not fully dissolved in the carrier at human body temperature. In one aspect, the pharmaceutical composition or unit dosage form is formulated as a tablet or capsule (e.g., soft gel or hard gel). In one aspect, the pharmaceutical composition or unit dosage form further comprises one or more additives (e.g., 2, 3, 4, 5, 6, 7, 8, 9, or 10). In one aspect, the pharmaceutical composition or unit dosage form comprises a particular amount of substantially pure (17-β)-3-Oxoandrost-4-en-17-yl undecanoate (e.g., 3 mg or more, 4 mg or more, 5 mg or more, 10 mg or more, 15 mg or more, 20 mg or more, 30 mg or more, 40 mg or more, 50 mg or more, 75 mg or more, 100 mg or more, 125 mg or more, 150 mg or more, 175 mg or more, 200 mg or more, 225 mg or more, 250 mg or more, 275 mg or more, or 300 mg or more) and typically less than 600 mg. In one aspect, the substantially pure (17-β)-3-Oxoandrost-4-en-17-yl undecanoate is substantially free of impurities specifically disclosed herein or in any of the Figures.

In one embodiment, the hydrophilic surfactant can comprise at least about 20% of the total pharmaceutical carrier. In another embodiment, the hydrophilic surfactant can comprise at least about 5 wt % of the carrier. In another embodiment, the hydrophilic surfactant can comprise less than 5, 4, 3, 2, or 1 wt % of the carrier.

In another embodiment, the composition or the dosage form includes or is prepared from substantially pure (17-β)-3-Oxoandrost-4-en-17-yl undecanoate, wherein the substantially pure (17-β)-3-Oxoandrost-4-en-17-yl undecanoate comprises about 0.5 wt % to about 50 wt %, 5 wt % to about 45 wt %, 15 wt % to about 40 wt %, 25 wt % to about 35 wt %, or 26 wt % to about 32 wt % of the composition or the dosage form, and wherein the composition includes about 50 wt % to about 100 wt % of lipophilic additive and 0 wt % to about 50 wt % of hydrophilic additive. In a specific embodiment, the lipophilic additive can be lipophilic surfactant and the hydrophilic additive can be hydrophilic surfactant. In one aspect, the lipophilic additive is a C16 to C18 fatty acid (saturated or having 1, 2, or 3 unsaturations), or a mono-, di-, or triglyceride thereof. In one aspect, mono-, di-, or tri glyceride is glyceryl palmitostearate. In one aspect, the hydrophilic component is a hydrogenated oil. In one aspect, the hydrophilic component is a polyoxylated hydrogenated oil. In a further embodiment, the substantially pure (17-β)-3-Oxoandrost-4-en-17-yl undecanoate is not solubilized at 30° C., or above 30° C., or at a temperature range above 30° C., including 30° C. to about 40° C. In an additional more specific embodiment, the substantially pure (17-β)-3-Oxoandrost-4-en-17-yl undecanoate is not fully dissolved in the lipophilic additive or the composition at human body temperature. In an additional more specific embodiment, the substantially pure (17-β)-3-Oxoandrost-4-en-17-yl undecanoate is not fully dissolved in the carrier at human body temperature. In one aspect, the pharmaceutical composition or unit dosage form is formulated as a tablet or capsule (e.g., soft gel or hard gel). In one aspect, the pharmaceutical composition or unit dosage form further comprises one or more additives (e.g., 2, 3, 4, 5, 6, 7, 8, 9, or 10). In one aspect, the pharmaceutical composition or unit dosage form comprises a particular amount of substantially pure (17-β)-3-Oxoandrost-4-en-17-yl undecanoate (e.g., 3 mg or more, 4 mg or more, 5 mg or more, 10 mg or more, 15 mg or more, 20 mg or more, 30 mg or more, 40 mg or more, 50 mg or more, 75 mg or more, 100 mg or more, 125 mg or more, 150 mg or more, 175 mg or more, 200 mg or more, 225 mg or more, 250 mg or more, 275 mg or more, or 300 mg or more) and typically less than 600 mg. In one aspect, the substantially pure (17-β)-3-Oxoandrost-4-en-17-yl undecanoate is substantially free of impurities specifically disclosed herein or in any of the Figures.

In one embodiment, the hydrophilic surfactant can comprise at least about 20% of the composition. In another embodiment, the hydrophilic surfactant can comprise at least about 5 wt % of the composition. In another embodiment, the hydrophilic surfactant can comprise less than 5 wt % of the composition.

In some embodiments, the oral pharmaceutical composition or the dosage form can include both a lipophilic surfactant and hydrophilic surfactant. In one embodiment, the lipophilic surfactant and hydrophilic surfactant can be present in amounts such that the ratio of amount (wt %) of lipophilic surfactant to amount (wt %) of hydrophilic surfactant is greater than 2:1. In another embodiment, the lipophilic surfactant and hydrophilic surfactant can be present in amounts such that the ratio of amount (wt %) of lipophilic surfactant to amount (wt %) of hydrophilic surfactant is greater than 2.5:1. In another embodiment, the lipophilic surfactant and hydrophilic surfactant can be present in amounts such that the ratio of amount (wt %) of lipophilic surfactant to amount (wt %) of hydrophilic surfactant is greater than 3.5:1. In still another embodiment, the lipophilic surfactant and hydrophilic surfactant can be present in amounts such that the ratio of amount (wt %) of lipophilic surfactant to amount (wt %) of hydrophilic surfactant is at least 6.5:1.

In one embodiment, the pharmaceutical composition has, on a weight to weight basis, from about 10% to about 50% of (17-β)-3-Oxoandrost-4-en-17-yl undecanoate and about 10% to about 90% of (a) octadecanoic acid, (9Z)-octadec-9-enoic acid, hexadecanoic acid or a combination thereof and (b) a mono-, di-, tri-propane-1,2,3-triol ester thereof, a combination of mono-, di-, or tri-propane-1,2,3-triol esters thereof or (c) a combination thereof. The pharmaceutical composition can be encapsulated e.g., soft gel or hard gel. The pharmaceutical composition may include a stabilizing agent, optional additive, or both.

Typically the fatty acids discussed herein, and their esters (e.g., of glycerides (a mono-, di-, tri-propane-1,2,3-triol ester) are “C8 to C22 fatty acid” and as referred to herein refers to both saturated fatty acids (e.g., having no carbon-carbon double bonds) and unsaturated fatty acids (e.g., having one or multiple carbon-carbon double bonds (e.g., 2, 3, 4, or 5 or more) wherein the molecule or moiety has from 8 to 22 carbons. Sub-ranges are also specified herein, for example, C16-C18 fatty acid refers to 16, 17, 18 carbon fatty acids. Examples of saturated C8 to C22 fatty acids include, but are not limited to and can be chosen from octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, and tetracosanoic acid. Examples of unsaturated C8 to C22 fatty acids can be chosen from, 9Z-octadecenoic acid, 5Z,8Z,11Z,14Z-eicosatetraenoic acid, 2Z-octenoic acid, 2E-octenoic acid, 3Z-octenoic acid, 3E-octenoic acid, 4Z-octenoic acid, 5Z-octenoic acid, 5E-octenoic acid, 6Z-octenoic acid, 6E-octenoic acid, 2Z-nonenoic acid, 3-nonenoic acid, 8-nonenoic acid, 2-decenoic acid, 3-decenoic acid, 4E-decenoic acid, 8-decenoic acid, 9-decenoic acid, 2-undecenoic acid, 9-undecenoic acid, 10-undecenoic acid, 2-dodecenoic acid, 4-dodecenoic acid, 6-dodecenoic acid, 7-dodecenoic acid, 9-dodecenoic acid, 10-dodecenoic acid, 11-dodecenoic acid, 2-tridecenoic acid, 11-tridecenoic acid, 12-tridecenoic acid, 2-tetradecenoic acid, 4-tetradecenoic acid, 8Z-tetradecenoic acid, 9Z-tetradecenoic acid, 2-pentadecenoic acid, 14-pentadecenoic acid, 2-hexadecenoic acid, 7-hexadecenoic acid, 9Z-hexadecenoic acid, 9E-hexadecenoic acid, 10Z-hexadecenoic acid, 2-heptadecenoic acid, 9Z-heptadecenoic acid, 2Z-octadecenoic acid, 2Z-octadecenoic acid, 3-octadecenoic acid, 4-octadecenoic acid, 5E-octadecenoic acid, 6Z-octadecenoic acid, 6E-octadecenoic acid, 7Z-octadecenoic acid, 7E-octadecenoic acid, 8Z-octadecenoic acid, 8E-octadecenoic acid, 9E-octadecenoic acid, 10Z-octadecenoic acid, 10E-octadecenoic acid, 11Z-octadecenoic acid, 11E-octadecenoic acid, 12Z-octadecenoic acid, 12E-octadecenoic acid, 15E-octadecenoic acid, 16E-octadecenoic acid, 17Z-octadecenoic acid, 2-nonadecenoic acid, 9Z-eicosenoic acid, 11Z-eicosenoic acid, 11E-eicosenoic acid, 14Z-eicosenoic acid, 11Z-docosenoic acid, 13Z-docosenoic acid, 13E-docosenoic acid, 22-tricosenoic acid, 15Z-tetracosenoic acid, 15E-tetracosenoic acid, 2E,4Z-decadienoic acid, 2E,4E-decadienoic acid, 2Z,6Z-decadienoic acid, 2E,6Z-decadienoic acid, 2E,6E-decadienoic acid, 4E,6E-decadienoic acid, 9,12-hexadecadienoic acid, 5Z,12Z-otadecadienoic acid, 5Z,12E-otadecadienoic acid, 5E,12Z-octadecadienoic acid, 5E,12E-octadecadienoic acid, 6, 8-octadecadienoic acid, 8E,10E-octadecadienoic acid, 8Z,11Z-octadecadienoic acid, 9Z,11Z-octadecadienoic acid, 9Z,11E-octadecadienoic acid, 9E,11E-octadecadienoic acid, 9Z,12Z-octadecadienoic acid, 9Z,12E-octadecadienoic acid, 9E,12Z-octadecadienoic acid, 9E,12E-octadecadienoic acid, 10Z,12Z-octadecadienoic acid, 10E,12Z-octadecadienoic acid, 10E,12E-octadecadienoic acid, 10Z,13Z-octadecadienoic acid, 10Z,13Z-nonadecadienoic acid, 11,14-eicosadienoic acid, 5,13-docosadienoic acid, 13,16-docosadienoic acid, 17,20-hexacosadienoic acid, 4,7,10-hexadecatrienoic acid, 5E,8E,11E-hexadecatrienoic acid, 6,9,12-hexadecatrienoic acid, 6,10,14-hexadecatrienoic acid, 7Z,10Z,13Z-hexadecatrienoic acid, 9,12,15-hexadecatrienoic acid, 3E,9Z,12Z-octadecatrienoic acid, 6Z,9Z,12Z-octadecatrienoic acid, 6,10,14-octadecatrienoic acid, 8Z,10E,12Z-octadecatrienoic acid, 8E,10E,12Z-octadecatrienoic acid, 8E,10E,12E-octadecatrienoic acid, 9Z,11E,13Z-octadecatrienoic acid, 9Z,11E,13E-octadecatrienoic acid, 9E,11E,13E-octadecatrienoic acid, 9,12,14-octadecatrienoic acid, 9Z,12Z,15Z-octadecatrienoic acid, 9E,12E,15E-octadecatrienoic acid, 10,12,14-octadecatrienoic acid, 10E,12E,14E-octadecatrienoic acid, 10,12,15-octadecatrienoic acid, 5,8,11-eicosatrienoic acid, 8Z,11Z,14Z-eicosatrienoic acid, 11,14,17-eicosatrienoic acid, 7,10,13-docosatrienoic acid, 8,11,14-docosatrienoic acid, 4Z,7Z,10Z,13Z-hexadecatetraenoic acid, 4,7,11,14-hexadecatetraenoic acid, 4,8,12,16-hexadecatetraenoic acid, 6,9,12,15-hexadecatetraenoic acid, 3E,9Z,12Z,15Z-octadecatetraenoic acid, 6,9,12,15-octadecatetraenoic acid, 9E,11E,13E,15E-octadecatetraenoic acid, 9,12,15,17-octadecatetraenoic acid, 4,8,12,16-eicosatetraenoic acid, 6,10,14,18-eicosatetraenoic acid, 8,11,14,17-eicosatetraenoic acid, 4,7,10,13-docosatetraenoic acid, 7Z,10Z,13Z,16Z-docosatetraenoic acid, 8,12,16,19-docosatetraenoic acid, 4,8,12,15,18-eicosapentaenoic acid, 4,7,10,13,16-docosapentaenoic acid, 4,8,12,15,19-docosapentaenoic acid, 7,10,13,16,19-docosapentaenoic acid, 4Z,7Z,10Z,13Z,16Z,19Z-docosahexaenoic acid, 4,8,12,15,19,21-tetracosahexaenoic acid, 5-(2-cyclopentenyl)-pentanoic acid, 7-(2-cyclopentenyl)-heptanoic acid, 9-(2-cyclopentenyl)-nonanoic acid, 3Z-decenoic acid, 4Z-decenoic acid, 2,3-decadienoic acid, 2,5-decadienoic acid, 2E,7E-decadienoic acid, 2E,7Z-decadienoic acid, 2Z,6E-decadienoic acid, 3,4-decadienoic acid, 3,5-decadienoic acid, 3E,5Z-decadienoic acid, 3Z,5E-decadienoic acid, 4,8-decadienoic acid, 4E,9-decadienoic acid, 5E,9-decadienoic acid, 5E,8E-decadienoic acid, 6E,8E-decadienoic acid, 7,9-decadienoic acid, 2E,6E,8E-decatrienoic acid, 2E,6Z,8E-decatrienoic acid, 2Z-undecenoic acid, 3E-undecenoic acid, 6Z-undecenoic acid, 8Z-undecenoic acid, 9Z-undecenoic acid, 2E,4E-undecadienoic acid, 10Z-dodecenoic acid, 2Z-dodecenoic acid, 5E-dodecenoic acid, 5Z-dodecenoic acid, 6Z-dodecenoic acid, 7Z-dodecenoic acid, 9Z-dodecenoic acid, 2E,4E-dodecadienoic acid, 2E,6Z-dodecadienoic acid, 2E,8E-dodecadienoic acid, 2E,8Z-dodecadienoic acid, 2Z,8E-dodecadienoic acid, 2Z,8Z-dodecadienoic acid, 5E,7E-dodecadienoic acid, 7Z,9E-dodecadienoic acid, 8E,10E-dodecadienoic acid, 8Z,10E-dodecadienoic acid, 2E,4E,8Z,10E-dodecatetraenoic acid, 2E,6E,8E,10E-dodecatetraenoic acid, 2E,6E,8Z,10E-dodecatetraenoic acid, 3,5,7,9,11-dodecapentaenoic acid, 3E,5E-tridecadienoic acid, 3Z,5E-tridecadienoic acid, 3E-tetradecenoic acid, 4Z-tetradecenoic acid, 5Z-tetradecenoic acid, 7Z-tetradecenoic acid, 9E-tetradecenoic acid, 10Z,12E-tetradecadienoic acid, 2E,4E-tetradecadienoic acid, 3,4-tetradecadienoic acid, 3Z,5E-tetradecadienoic acid, 3Z,5Z-tetradecadienoic acid, 5,8-tetradecadienoic acid, 5Z,8Z-tetradecadienoic acid, 5,7,9,11,13-tetradecapentaenoic acid, 10Z-pentadecenoic acid, 10-hexadecenoic,11-hexadecenoic acid, 11Z-hexadecenoic acid, 13-hexadecenoic acid, 13Z-hexadecenoic acid, and 2Z-hexadecenoic acid. It is noted that the term C8 to C22 is intended to include both branched chain versions, cyclic versions, as well as straight versions as long as the total number of carbons of the fatty acid is in the range of from 8 carbons to 22 carbons. Preferred amongst the C8 to C22 fatty acids described in this paragraph are those that are pharmaceutically acceptable. Even more preferred amongst the C8 to C22 fatty acids described in this paragraph are those that are pharmaceutically acceptable to one or more regulatory agencies such as the United States Food and Drug Agency (e.g., inactive ingredients in approved drug products; or 1. determination by FDA that the substance is “generally recognized as safe” (GRAS) pursuant to Title 21, U.S. Code of Federal Regulations, Parts 182, 184 or 186 (21 CFR 182, 184 & 186), 2. approval of a food additive petition as set forth in 21 CFR 171; or 3. the excipient is referenced in, and part of, an approved new drug application (NDA) for a particular function in that specific drug product), the European Medicines Agency, the Japanese Pharmaceuticals and Medical Devices, or is listed in a Pharmacopoeia (e.g., European, United States, Japan, International, or one in the World Health Organization Index of Pharmacopoeias).

In one aspect, preferred C8 to C22 fatty acids are chosen from C14 to C20 fatty acids. In one aspect, preferred C8 to C22 fatty acids are selected from octadecanoic acid, (9Z)-octadec-9-enoic acid, hexadecanoic, (9Z,12Z)-9,12-octadecadienoic acid, or hexadec-9-enoic acid. In one aspect, preferred C8 to C22 fatty acids are selected from octadecanoic acid, (9Z)-octadec-9-enoic acid, hexadecanoic or hexadec-9-enoic acid.

As used herein, “a mono-, di-, tri-propane-1,2,3-triol ester” refers to a fatty acid ester or fatty acid esters (e.g., two or more (combination of esters or mixtures of esters)) of propane-1,2,3-triol. It is noted that the same propane triol can have different ester moieties (derived from chemically distinct fatty acids). In this context fatty acid is as defined above e.g., C8 to C22 fatty acids. In a specific definition, C8 to C22 fatty acids that form the ester moiety or moieties of the mono-, di-, propane triol ester are selected from octadecanoic acid, (9Z)-octadec-9-enoic acid, hexadecanoic acid, (9Z,12Z)-9,12-octadecadienoic acid, or hexadec-9-enoic acid. In another specific definition, the mono-, di-, tri-propane-1,2,3-triol ester is 1-octadecanoyl-2-hexadecanoyl-glycerol, 1-octadecanoyl-3-hexadecanoyl-glycerol, 1-hexadecanoyl-2-octadecanoyl-glycerol, 1,2-dioctadecanoyl-glycerol, 1,3-dioctadecanoyl-glycerol, 1,2-dihexadecanoyl-glycerol, 1,3-dihexadecanoyl-glycerol, 1, 2, 3-trihexadecanoyl-glycerol, 1, 2, 3-trioctadecanoyl-glycerol, 1,2-dioctadecanoyl-3-hexadecanoyl-glycerol, 1,3-dioctadecanoyl-2-hexadecanoyl-glycerol, 1,2-dihexadecanoyl-3-octadecanoyl-glycerol, or 1,3-dihexadecanoyl-2-octadecanoyl-glycerol. In another specific aspect, at least 50%, 60%, 70%, 80%, or 85% of the total content of mono-, di-, tri-propane-1,2,3-triol ester is 1-octadecanoyl-2-hexadecanoyl-glycerol, 1-octadecanoyl-3-hexadecanoyl-glycerol, 1-hexadecanoyl-2-octadecanoyl-glycerol, 1,2-dioctadecanoyl-glycerol, 1,3-dioctadecanoyl-glycerol, 1,2-dihexadecanoyl-glycerol, 1,3-dihexadecanoyl-glycerol, 1, 2, 3-trihexadecanoyl-glycerol, 1, 2, 3-trioctadecanoyl-glycerol, 1,2-dioctadecanoyl-3-hexadecanoyl-glycerol, 1,3-dioctadecanoyl-2-hexadecanoyl-glycerol, 1,2-dihexadecanoyl-3-octadecanoyl-glycerol, 1,3-dihexadecanoyl-2-octadecanoyl-glycerol, or a combination thereof. In another specific definition the combination of mono-, di-, tri-propane-1,2,3-triol ester are esters of octadecanoic acid, hexadecanoic acid, or a combination thereof. In another specific definition the combination of mono-, di-, tri-propane-1,2,3-triol ester are esters of octadecanoic acid, hexadecanoic acid, or a combination thereof with a triglyceride content of from 30% to 60%, a diglyceride content of 25% to 55% and a monoglyceride content of 4% to 15%.

In specific embodiments and compositions described herein, free fatty acids are employed (e.g., as a carrier or solvent for API). When such compounds are referred to herein, it is understood that such compounds often are not 100% one particular free fatty acid. Thus, reference to a specific free fatty acid refers to a compound that is at least 50% of a specific free fatty acid by weight, preferably at least 55%, 60%, 65%, 70%, 75%, 80%, 85% or 90%. In one specific, the fatty acid is (9Z)-octadec-9-enoic acid which is at least 55%, 60%, 65%, 70%, 75%, 80%, 85% or 90% (9Z)-octadec-9-enoic acid. For example, at least 65% or at least 85% (9Z)-octadec-9-enoic acid. In one specific aspect, (9Z)-octadec-9-enoic acid is about 63%-100% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In another specific aspect, (9Z)-octadec-9-enoic acid is about 70%-100% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In one specific aspect, (9Z)-octadec-9-enoic acid is about 80%-100% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In one specific aspect, (9Z)-octadec-9-enoic acid is about70%-95% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In one specific aspect, (9Z)-octadec-9-enoic acid is about 75%-95% (9Z)-octadec-9-enoic acid, less than 4% tetradecanoic acid, less than 14% hexadecanoic acid, less than 6% (9Z)-hexadec-9-enoic acid, less than 4% octadecanoic acid, less than 16% (9Z,12Z)-9,12-octadecadienoic acid, less than 4% linolenic acid and less than 3% fatty acid with chain length greater than 18 carbons. In a specific aspect, (9Z)-octadec-9-enoic acid has one or more of the following, more than 0.1% tetradecanoic acid, more than 0.1% hexadecanoic acid, more than 0.1% (9Z)-hexadec-9-enoic acid, more than 0.1% octadecanoic acid, more than 0.1% (9Z,12Z)-9,12-octadecadienoic acid, more than 0.1% linolenic acid and more than 0.1% fatty acid with chain length greater than 18 carbons. In a specific aspect, (9Z)-octadec-9-enoic acid is greater than 80 or 85% (9Z)-octadec-9-enoic acid has one or more of the following, 0.1-5% tetradecanoic acid, 0.1-16%% hexadecanoic acid, 0.1-8% (9Z)-hexadec-9-enoic acid, 0.1-6% octadecanoic acid, more than 0.118% (9Z,12Z)-9,12-octadecadienoic acid, 0.1-4% linolenic acid and 0.1-4% fatty acid with chain length greater than 18 carbons. In a specific aspect, (9Z)-octadec-9-enoic acid has a melting point in the range of about 4-14° C. or about 6-12° C. In a specific aspect, (9Z)-octadec-9-enoic acid is from a vegetable source. In a specific aspect, (9Z)-octadec-9-enoic acid is from an edible source.

It has been found that (9Z)-octadec-9-enoic acid preparation can affect the stability and performance of oral pharmaceutical composition comprising this carrier. Thus, the formulation, compositions and dosage forms described herein that contain (9Z)-octadec-9-enoic acid preferably contain a preparation as described in the Table below and subsequent two paragraphs.

TABLE
Different percent content of
components of (9Z)-octadec-9-enoic acid
(ratio of (9Z)-Octadec-9-enoic acid to other fatty acid in parentheses)

| | | | | (9Z)- | | | | |
|---|---|---|---|---|---|---|---|---|
| | (9Z)- | Tetra- | Hexa- | Hexadec- | | (9Z,12Z)- | | C18+ |
| | Octadec- | decanoic | decanoic | 9-enoic | Octa- | 9,12-Octa- | Linolenic | Fatty |
| | 9-enoic | acid | acid | acid | decanoic | decadienoic | acid | Acid |
| | acid % | % | % | (%) | acid % | acid % | % | % |
| 1 | 50 | 5 | 2 | 5 | 20 | 10 | 5 | 3 |
| | | | | | (2.5:1) | | | |
| 2 | 55 | 4 | 10 | 6 | 15 | 5 | 3 | 2 |
| | | | | | (3.67:1) | | | |
| 3 | 60 | 3 | 23 | 2 | 2 | 5 | 3 | 2 |
| | | | (2.6:1) | | | | | |
| 4 | 65 | 3 | 7 | 5 | 10 | 4 | 3 | 3 |
| | | | | | (6.5:1) | | | |
| 5 | 75 | 1 | 2 | 3 | 15 (5:1) | 2 | 1 | 1 |
| 6 | 65 | 3 | 8 | 3 | 10 | 4 | 3 | 4 |
| | | | | | (6.5:1) | | | |
| 7 | 65 | 3 | 10 | 4 | 3 | 7 | 2 | 1 |
| 8 | 75 | 2 | 3 | 4 | 1 | 9 | 3 | 3 |
| 9 | 80 | 3 | 3 | 1 | 6 | 2 | 4 | 1 |
| 10 | 85 | 2 | 2 | 3 | 4 | 2 | 1 | 1 |
| 11 | 86 | 2 | 2 | 2 | 2 | 2 | 2 | 2 |
| 12 | 88 | 1 | 1 | 1 | 6 | 1 | 1 | 1 |
| 13 | 90 | 1 | 1 | 2 | 3 | 1 | 1 | 1 |
| 14 | 93 | 1 | 1 | 1 | 1 | 1 | 1 | 1 |
| 15 | 95 | 0 | 1 | 1 | 1 | 1 | 1 | 0 |
| 16 | 70 | 0 | 0 | 0 | 0 | 23 (3:1) | 7 | 0 |
| | | | | | | | (10:1) | |
| 17 | 68 | 0 | 0 | 0 | 0 | 22 (3.1:1) | 10 | 0 |
| | | | | | | | (6.8:1) | |

In the Table above, numbers 7-15 are considered to have desirable properties whereas numbers 1-6 are considered to have undesirable properties. Undesirable properties include, but are not limited to, undesirable dissolution or release profiles (or stability thereof) of API from formulations containing (9Z)-octadec-9-enoic acid, undesirable stability of API in formulation containing (9Z)-octadec-9-enoic acid, undesirable solubility of API in formulations containing (9Z)-octadec-9-enoic acid, undesirable manufacturing properties for formulations containing (9Z)-octadec-9-enoic acid or a combination thereof.

In some embodiments, it was also found that particular ratios of components of (9Z)-octadec-9-enoic acid are desirable or undesirable (e.g., having desirable or undesirable properties such as those described in the paragraph above). For example, a ratio (9Z)-octadec-9-enoic acid:Octadecanoic acid of 10:1 or greater is desirable; a ratio (9Z)-octadec-9-enoic acid:hexadecanoic acid of 4:1 or greater is desirable; a ratio of unsaturated:unsaturated C14-C18 fatty acid of greater than 2:1 is desirable. In related examples, a ratio of (9Z)-octadec-9-enoic acid:octanoic acid of greater than 4:1 is desirable; a ratio of (9Z)-octadec-9-enoic acid:octanoic acid of less than 4:1 is undesirable; a ratio of (9Z)-octadec-9-enoic acid:hexanoic acid of greater than 4:1 is desirable; a ratio of (9Z)-octadec-9-enoic acid:decanoic acid of greater than 4:1 is desirable. It has also been found that higher ratios of (9Z)-Octadec-9-enoic acid to (9Z,12Z)-9,12-Octa-decadienoic acid, linolenic acid or both are desirable as increased levels of components lead to undesirable properties such as chemical instability of API, physical instability of the composition or unit dosage form, undesirable dissolution or release profiles (or stability thereof) of API from formulations or a combination thereof. For example, a ratio of (9Z)-octadec-9-enoic acid: (9Z,12Z)-9,12-Octa-decadienoic acid of less than 4:1, 3.8:1, or 3.5:1 is undesirable. For example, a ratio of (9Z)-octadec-9-enoic acid:linolenic acid of less than 20:1, 17:1, or 15:1 is undesirable. Thus, Examples 16 and 17 in the Table above are undesirable.

In one embodiment, the (17-β)-3-Oxoandrost-4-en-17-yl undecanoate as described in this paragraph is further part of a composition having one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; H—(O—CH₂—CH₂)ₙ—OH where n is an integer from 3 to 900; a branched; star, or comb analog of H—(O—CH₂—CH₂)ₙ—OH where n is an integer from 3 to 900. In a more specific aspect of this embodiment, the (17-β)-3-Oxoandrost-4-en-17-yl undecanoate as described in this paragraph are further part of a composition having one or more components chosen from the following:

(A) octadecanoic acid, (9Z)-octadec-9-enoic acid, (9Z,12Z)-9,12-octadecadienoic acid, or hexadecanoic acid;

(B) a mono-, di-, or tri-propane-1,2,3-triol ester of (A);

(C)a combination of mono-, di-, or tri-propane-1,2,3-triol esters of (A);

(D) a combination of one or more of (A)-(C);

(E) (2S,5R)-2-Isopropyl-5-methylcyclohexanone, acetic acid [(1R,2S,5R)-2-isopropyl-5-methylcyclohexyl] ester; (1R,2S,5R)-2-isopropyl-5-methylcyclohexanol or a combination thereof;

(F) 2-isopropyl-5-methylcyclohexanol, 2-Isopropyl-5-methylcyclohexanone, acetic acid [(2-isopropyl-5-methylcyclohexyl] ester or a combination thereof.

(G) Polyoxyethylated oil;

(H) Polyoxyethylated hydrogenated vegetable oil;

(I) Polyoxyethylated hydrogenated vegetable oil;

(J) polyoxyethylated hydrogenated castor oil;

(K) H—(O—CH₂—CH₂)ₙ—OH where n is an integer from 5 to 600;

(L) a branched; star, or comb analog (in this specific context analog refers to a molecule having the same molecular weight or average molecular weight) of H—(O—CH₂—CH₂)ₙ-—OH where n is an integer from 5 to 600; and

(M) polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-poly(propylene oxide)-poly(ethylene oxide), polyvinyl alcohol, polystyrenesulfonic acid, polyvinylpyrrolidone-co-polyvinyl acetate, polyether polyol, carboxymethylcellulose, methylcellulose, hydroxyethyl cellulose, hydroxypropylmethyl cellulose phthalate, hydroxypropylmethyl cellulose acetate succinate, or a combination thereof. The pharmaceutical composition has ((17-β)-3-Oxoandrost-4-en-17-yl undecanoate at greater than 23%, 24%, 25%, 26%, 27%, 28%, or 29% w/w drug loading (and less than 50%, 40%, 35%, 33%, or 32%). For example, daily doses sufficient to provide substantial therapeutic effects in hypogonadal males e.g., serum (8R,9S,10R,13S,14S,17S)-17-Hydroxy-10,13-dimethyl-1,2,6,7,8,9,11,12,14,15,16,17-dodecahydrocyclopenta[a]phenanthren-3-one levels (e.g., Cavgl-24) of greater than 300 ng/dL, can be achieved by the administration of 1, 2, 3, 4, 5, or 6 unit dosage forms as described herein. The unit dosage forms described herein can have 125 mg or more, 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg. The formulations also are release profile stable and have a suitable release profile (e.g., at least 50% in two hours). The formulations may also include an optional additive as described below in the following paragraphs. In one aspect, the composition is formulated as a capsule e.g., soft gel or hard gel.

In some embodiments, the composition described herein comprises (17-β)-3-Oxoandrost-4-en-17-yl undecanoate (at greater than 23%, 24%, 25%, 26%, 27%, 28%, or 29% w/w drug loading (and less than 50%, 40%, 35%, 33%, or 32%) and one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; H—(O—CH₂—CH₂)ₙ—OH where n is an integer from 3 to 900; a branched; star, or comb analog of H—(O—CH₂—CH₂)ₙ—OH where n is an integer from 3 to 900 when tested in a USP type 2 paddle apparatus having 1000 mL of 8% Octoxynol-9 (Triton-X100) in water at 37° C. (±0.5) (a) releases 80% or more of the active pharmaceutical ingredient at 12, 11, 10, 9, 8, 7, 6, 5, 4, 3, 2, 1, 0.5, or 0.25 hours (b) releases less than 100% at 12, 11, 10, 9, 8, 7, 6, 5, 4, 3, 2, 1, 0.5, or 0.25 hours (c) releases about 100% at 12, 11, 10, 9, 8, 7, 6, 5, 4, 3, 2, 1, 0.5, or 0.25 hours or (d) a combination of one, two, or three of (a)-(c). In one aspect, the composition of this embodiment releases (a) at least 80% or more at 0.25, 0.5, 1, 2, 3, or 4 hours; (b) less than 100% at 6, 5, 4, 3, 2, 1, 0.5, or 0.25 hours; (c) about 100% at 8, 7, 6, 5, 4, 3, 2, 1, 0.5, or 0.25 hours or (d) a combination of one, two, or three of (a)-(c). In one aspect, the composition of this embodiment releases (a) at least 80% or more at 0.25, 0.5, 1, or 2 hours; (b) less than 100% at 3, 2, 1, 0.5, or 0.25 hours; (c) about 100% at 4, 3, 2, 1, 0.5, or 0.25 hours or (d) a combination of one, two, or three of (a)-(c). In one aspect, the composition of this embodiment releases (a) at least 75% or more at 2 hours and (b) less than 95% or 90% at 0.25 hours. In certain aspects of this embodiment, the composition comprises from about 23% to about 35% (17-β)-3-Oxoandrost-4-en-17-yl undecanoate active pharmaceutical ingredient and from 20% to 77% of one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; H—(O—CH₂—CH₂)ₙ—OH where n is an integer from 3 to 900; a branched; star, or comb analog of H—(O—CH₂—H₂)ₙ—OH where n is an integer from 3 to 900. In a specific aspect, of this paragraph, the composition comprises 23%-35% (0713)-3-Oxoandrost-4-en-17-yl undecanoate active pharmaceutical ingredient and 20%-77% one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; H—(O—CH₂—CH₂)ₙ—OH where n is an integer from 3 to 900; a branched; star, or comb analog of H—(O—CH₂—CH₂)ₙ—OH where n is an integer from 3 to 900; polyoxyethylated oil; polyoxyethylated hydrogenated vegetable oil; polyoxyethylated hydrogenated vegetable oil; and polyoxyethylated hydrogenated castor oil. In another specific aspect, the composition comprises 23%-35% of the active pharmaceutical ingredient and 20%-77% one or more components chosen from the following a C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid. In another specific aspect, the composition comprises 23%-35% lipophilic tetracyclic active pharmaceutical ingredient and 20%-77% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C14 to C20 fatty acid. In another specific aspect, the composition comprises 23%-35% the active pharmaceutical ingredient and 20%-77% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C4 to C20 fatty acid. In another specific aspect, the composition comprises 25%-35% of the active pharmaceutical ingredient and 25%-75% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C14 to C20 fatty acid. In another specific aspect, the composition comprises 25%-35% the active pharmaceutical ingredient and 30%-75% one or more components chosen from the following a C14 to C20 fatty acid; a mono-, di-, tri-propane-1,2,3-triol ester of a C14to C20 fatty acid; a combination (e.g., mixture) of mono-, di-, tri-propane-1,2,3-triol esters of a C14 to C20 fatty acid. In another specific aspect, the composition comprises 25%-35% of the active pharmaceutical ingredient and 40%-75% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C14 to C20 fatty acid. In another specific aspect, the composition comprises 23%-35% of the active pharmaceutical ingredient and 20%-77% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 23%-35% of the active pharmaceutical ingredient and 25%-77% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 25%-75% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 30%-75% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 40%-75% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 40%-75% one or more components chosen from the following (A) octadecanoic acid, (9Z)-octadec-9-enoic acid, hexadecanoic or a combination thereof; (B) a mono-, di-, tri-propane-1,2,3-triol ester of (A); (C)a combination of mono-, di-, or tri-propane-1,2,3-triol esters of (A); and (D) combination of one or more of (A)-(C). According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil; or a combination thereof and is present in an amount (w/w) of from 0% to 25%, 1% to 25%, 0% to 15%, 1% to 15%, 0% to 10%, 1% to 10%, 0% to 8%, 1% to 8%, less than 5%, less than 4%, less than 3%, or less than 2%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In one embodiment, the composition comprises (17-β)-3-Oxoandrost-4-en-17-yl undecanoate (at greater than 23%, 24%, 25%, 26%, 27%, 28%, or 29% w/w drug loading (and less than 50%, 40%, 35%, 33%, or 32%) and one or a combination of mono-, di-, or tri-fatty acid esters of propane-1,2,3-triol. In a specific aspect, the one or a combination of mono-, di-, or tri-fatty acid esters of propane-1,2,3-triol are esters of one or more of octadecanoic acid or hexadecanoic acid. In one aspect, one or combination of mono-, di-, or tri-fatty acid esters of propane-1,2,3-triol is an additive that allows for loading of the API in the formulation above its solubility limit without substantially compromising release profile, release profile stability, bioavailability or a combination thereof. In one aspect, the total mono-ester content is from about 0% to about 50%. In one aspect, the total mono-ester content is from about 2% to about 50%. In one aspect, the total mono-ester content is from about 3% to about 40%. In one aspect, the total mono-ester content is from about 4% to about 35%. In one aspect, the total di-ester content is from about 0% to about 90%. In one aspect, the total di-ester content is from about 10% to about 90%. In one aspect, the total di-ester content is from about 20% to about 80%. In one aspect, the total di-ester content is from about 25% to about 75%. %. In one aspect, the total tri-ester content is from about 0% to about 90%. In one aspect, the total tri-ester content is from about 5% to about 80%. In one aspect, the total tri-ester content is from about 15% to about 70%. In one aspect, the total tri-ester content is from about 15% to about 60%. In one aspect, the total tri-ester content is from about 15% to about 50%. In one aspect, the ester content is from about 10% to about 90% octadecanoic acid. In one aspect, the ester content is from about 20% to about 80% octadecanoic acid. In one aspect, the ester content is from about 25% to about 75% octadecanoic acid. In one aspect, the ester content is from about 30% to about 70% octadecanoic acid. In one aspect, the ester content is from about 10% to about 90% hexadecanoic acid. In one aspect, the ester content is from about 20% to about 80% hexadecanoic acid. In one aspect, the ester content is from about 25% to about 75% hexadecanoic acid. In one aspect, the ester content is from about 30% to about 70% hexadecanoic acid. In one aspect, the ester content is from about 30% to about 70% hexadecanoic acid; about 30% to about 70% octadecanoic acid; mono-ester content is from about 4% to about 35%; di-ester content is from about 25% to about 75%; and tri-ester content is from about 15% to about 50%. In one aspect, the melting point of the combination of mono-, di-, or tri-fatty acid esters of propane-1,2,3-triol is in the range of from about 30° C. to 100° C. In another aspect, the melting point of the combination of mono-, di-, or tri-fatty acid esters of propane-1,2,3-triol is in the range of from about 35° C. to 90° C. In another aspect, the melting point of the combination of mono-, di-, or tri-fatty acid esters of propane-1,2,3-triol is in the range of from about 40° C. to 80° C. In another aspect, the melting point of the combination of mono-, di-, or tri-fatty acid esters of propane-1,2,3-triol is in the range of from about 40° C. to 70° C. In another aspect, the melting point of the combination of mono-, di-, or tri-fatty acid esters of propane-1,2,3-triol is in the range of from about 45° C. to 65° C. Thus, according to one aspect of this embodiment, a composition is provided that has from about 0.1% to about 25% w/w of a combination of mono-, di-, or tri-fatty acid esters of propane-1,2,3-triol ; from about 10% to about 40% w/w active pharmaceutical ingredient; from about 20% to about 75% fatty acid; and optionally, one or more pharmaceutically acceptable excipients. According to another aspect of this embodiment, a composition is provided that has from about 1% to about 20% w/w of a combination of mono-, di-, or tri-fatty acid esters of propane-1,2,3-triol ; from about 20% to about 40% w/w active pharmaceutical ingredient; from about 20% to about 75% fatty acid; and optionally, one or more pharmaceutically acceptable excipients. According to yet another aspect of this embodiment, a composition is provided that has from about 1% to about 20% w/w of a combination of mono-, di-, or tri-fatty acid esters of propane-1,2,3-triol ; from about 25% to about 40% w/w active pharmaceutical ingredient; from about 20% to about 75% fatty acid; and optionally, one or more pharmaceutically acceptable excipients. According to again another aspect of this embodiment, a composition is provided that has from about 1% to about 20% w/w of a combination of mono-, di-, or tri-fatty acid esters of propane-1,2,3-triol ; from about 25% to about 35% w/w pharmaceutical ingredient; from about 30% to about 75% fatty acid; and optionally, one or more pharmaceutically acceptable excipients. In an alternative aspect, the fatty acid is octadecanoic acid, (9Z)-octadec-9-enoic acid, (9Z,12Z)-9,12-octadecadienoic acid or hexadecanoic acid. In another alternative aspect, the fatty acid is a C16-C18 fatty acid. According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil;

or a combination thereof and is present in an amount (w/w) of from 0% to 25%, 0% to 15%, 0% to 10% or 1% to about 8%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In some embodiments, the composition described herein has a compound of formula: H—(O—CH₂—CH₂)ₙ—OH where n is an integer from 5 to 2000 and comprises (17-β)-3-Oxoandrost-4-en-17-yl undecanoate (at greater than 23%, 24%, 25%, 26%, 27%, 28%, or 29% w/w drug loading (and less than 50%, 40%, 35%, 33%, or 32%). The compound of formula: H—(O—CH₂—CH₂)ₙ—OH cans serve as a stabilizing agent that prevents or inhibits crystallization of API, allows for increased loading of API without substantially compromising the release profile, release profile stability, bioavailability or a combination thereof. In some aspects of this embodiment, n is an integer from 9 to 1000. In some aspects of this embodiment, n is an integer from 9 to 1000. In some aspects of this embodiment, n is an integer from 9 to 500. In some aspects of this embodiment, n is an integer from 9 to 500. In some aspects, the compound of formula: H—(O—CH₂—CH₂)ₙ—OH is characterized as having an average molecular weight of about 100 to about 50,000 gram/mol. In some aspects, the compound of formula: H—(O—CH₂—CH₂)ₙ—OH is characterized as having an average molecular weight of about 200 to about 30,000 gram/mol. In some aspects, In some aspects, the melting point of from about 30 C to about 100 C is characterized as having an average molecular weight of about 300 to about 20,000 gram/mol. In some aspects, the compound of formula: H—(O—CH₂—CH₂)ₙ—OH is characterized as having an average molecular weight of about 400 to about 20,000 gram/mol. In some aspects, the compound of formula: H—(O—CH₂—CH₂)ₙ—OH is characterized as having an average molecular weight of about 600 to about 15,000 gram/mol. In some aspects, the compound of formula: H—(O—CH₂—CH₂)ₙ—OH has a melting point of from about 4° C. to about 150° C. In some aspects, the melting point of from about 10° C. to about 100° C. In some aspects, the compound of formula: H—(O—CH₂—CH₂)ₙ—OH has a melting point of from about 20° C. to about 100° C. In some aspects, the compound of formula: H—(O—CH₂—CH₂)ₙ—OH has a melting point of from about 25° C. to about 100° C. In some aspects, the compound of formula: H—(O—CH₂—CH₂)ₙ—OH has a melting point of from about 20° C. to about 70° C. In some aspects, the compound of formula: H—(O—CH₂—CH₂)ₙ—OH has a melting point of from about 25° C. to about 60° C. In some aspects, the compound of formula: H—(O—CH₂—CH₂)ₙ—OH has a melting point of from about 30° C. to about 60 ° C. Thus, according to one aspect of this embodiment, a composition is provided having 0.1% to about 30% of a compound of formula: H—(O—CH₂—CH₂)ₙ—OH where n is an integer from 5 to 2000; from about 20% to about 40% w/w active pharmaceutical ingredient; from about 20% to about 75% fatty acid (e.g., C16-C18), from about 0% to about 20% w/w of a mono-, di-, or tri-fatty acid esters of propane-1,2,3-triol or a combination thereof; and optionally, one or more pharmaceutically acceptable excipients. In a specific aspect, the fatty acid is a C16 to C18 fatty acid. In a specific aspect, the fatty acid is octadecanoic acid, (9Z)-octadec-9-enoic acid, or hexadecanoic acid. In a specific aspect, the mono-, di-, or tri-fatty acid esters of propane-1,2,3-triol or a combination thereof are esters of octadecanoic acid, (9Z)-octadec-9-enoic acid, or hexadecanoic acid. In a specific aspect, the compound of formula: H—(O—CH₂—CH₂)ₙ—OH has a molecular weight of about 800 to 12000 gram/mol. In one aspect of this embodiment the active pharmaceutical ingredient is present in the composition in an amount of from about 10% to about 40% w/w. In another aspect of this embodiment the active pharmaceutical ingredient is present in the composition in an amount of from about 20% to 40% w/w. In again another aspect of this embodiment, the active pharmaceutical ingredient is present in the composition in an amount of from about 25% to 35% w/w. According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil; or a combination thereof and is present in an amount (w/w) of from 0% to 25%, 0% to 15%, 0% to 10% or 1% to about 8%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In some embodiments, the composition described herein comprises (17-β)-3-Oxoandrost-4-en-17-yl undecanoate (at greater than 23%, 24%, 25%, 26%, 27%, 28%, or 29% w/w drug loading (and less than 50%, 40%, 35%, 33%, or 32%) and one or more of 2-isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof. Accordingly, in a specific aspect, the composition comprises 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof, in an amount ranging from about 5% to about 40% (w/w). In another specific aspect, the pharmaceutical composition (e.g., unit dosage form, formulation, or pharmaceutical composition) comprises from about 5% to about 40% (w/w) of a composition having 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof, where 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof, is at least 10%, 20%, or 25% (1R,2S,5R)-2-isopropyl-5-methylcyclohexanol and at least 5%, 8%, or 12% (2S,5R)-2-Isopropyl-5-methylcyclohexanone. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In one embodiment, the composition has, on a weight to weight basis, from about 20% to about 50%, 23% to 35%, or 26% to 32% of (17-β)-3-Oxoandrost-4-en-17-yl undecanoate and about 10% to about 90% of (a) one or more pharmaceutically acceptable excipients. According to this embodiment, the pharmaceutically acceptable excipients and amounts of (17-β)-3-Oxoandrost-4-en-17-yl undecanoate are selected such that they allow for once or twice daily dosing of one, two, or three unit dosage forms to provide Cavg1-24 serum testosterone values in hypogonadal males of about 300 ng/dL, 350 ng/dL, or 450 ng/dL or more. In some aspects of this embodiment, the composition is formulated such that Cavg1-24 values are at least 500 ng/dL, 550 ng/dL, 600 ng/dL or 650 ng/dL. In some aspects of this embodiment, the composition is formulated such that it minimizes supraphysiological serum testosterone levels. In a specific aspect, minimizes supraphysiological serum testosterone levels refers to greater than 2500 ng/dL in 1% or less of a population of patients, greater than 1800 ng/dL and less than 2500 ng/dL in less than 5% of a population of patients, or less than 1500 ng/dL in greater than 85% of a population of patients. As used herein a population can refer to 10-20, 20-50, or 50-100 individuals. In a specific aspect of this embodiment, the composition is formulated as a unit dosage forms and has greater than about 150 mg of (17-β)-3-Oxoandrost-4-en-17-yl undecanoate. In another specific aspect, the composition is formulated as a unit dosage form and has greater than about 160 mg, 170 mg, 160 mg, 180 mg, 190 mg, 200 mg, 210 mg, 220 mg, 230 mg, 240 mg, 250 mg, 260 mg, 270 mg, 280 mg, 290 mg or 300 mg of (17-β)-3-Oxoandrost-4-en-17-yl undecanoate. In another specific aspect, the composition is formulated as a unit dosage form and has less than about 600 mg, 500 mg, 475 mg, 450 mg, 440 mg, 430 mg, 420 mg, 410 mg, 400 mg, 390 mg, 380 mg, 370 mg, 360 mg, 350 mg, 340 mg or 330 mg of (17-β)-3-Oxoandrost-4-en-17-yl undecanoate. In another specific aspect, the composition is formulated as a unit dosage form and has greater than about 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 360 mg, 380 mg, 390 mg, 400 mg, 410 mg, 420 mg, 430 mg, 440 mg, 450 mg, 460 mg, 470 mg, 480 mg, 490 mg or 500 mg of pharmaceutically acceptable excipient(s). In another specific aspect, the composition is formulated as a unit dosage form and has less than about 1000 mg, 950 mg, 900 mg, 850 mg, 800 mg, 750 mg, 700 mg, 650 mg, 600 mg, or 550 mg of pharmaceutically acceptable excipients. In one aspect of this embodiment, the composition is not a liquid at 5° C.; a gel, paste, or semi-solid at 15° C.; a liquid at 25, 30, 35 or 40° C.; or a combination thereof. In one aspect of this embodiment, one or more of the pharmaceutically acceptable excipients are (a) octadecanoic acid, (9Z)-octadec-9-enoic acid or hexadecanoic acid or (b) a mono-, di-, tri-propane-1,2,3-triol ester thereof, a combination of mono-, di-, or tri-propane-1,2,3-triol esters thereof or (c) any combination thereof. In some aspects, the pharmaceutically acceptable excipient may also be selected from or include 2-Isopropyl-5-methylcyclohexanone; (2S, 5R)-2-Isopropyl-5-methylcyclohexanone; acetic acid [(1R,2S, 5R)-2-isopropyl-5-methylcyclohexyl] ester; acetic acid [(2-isopropyl-5-methylcyclohexyl] ester; (1R,2S, 5R)-2-isopropyl-5-methylcyclohexanol; 2-isopropyl-5-methylcyclohexanol; or a combination thereof. In another aspect, the pharmaceutically acceptable excipient may also be chosen from a polyoxyethylated oil; a polyoxyethylated hydrogenated vegetable oil; a polyoxyethylated hydrogenated castor oil; a compound of formula H—(O—CH₂—CH₂)ₙ—OH where n is an integer from 5 to 600; or a combination thereof. The formulations also are release profile stable. The formulations may also include an optional additive as described above. In one aspect, the composition is formulated as a capsule e.g., soft gel or hard gel.

The composition or dosage form as described herein can optionally include any of the following additives (some of which as the skilled artisan recognizes will act as the stabilizing agent or additive that allows for the production of compositions having the beneficial properties described herein) in the following seven paragraphs as long as the properties of the composition or dosage form are consistent with those as described herein.

Exemplary Soft Gel Capsule Formulations

Provided in this section are exemplary soft gel capsules or soft gel capsule fill formulations having from about 20% to about 50%w/w, 23% to 35% w/w, or 26% to 32% w/w, an active pharmaceutical ingredient which is (17-β)-3-Oxoandrost-4-en-17-yl undecanoate and a pharmaceutically acceptable carrier which is a carrier for the active pharmaceutical ingredient. More specifically, the carrier is a solvent for the active pharmaceutical ingredient. Typically, these formulations are non-solid at room temperature. Desirably, the carrier allows for high concentrations of the active pharmaceutical ingredient without substantially compromising bioavailability. In some specific aspects, the carrier also includes one or more pharmaceutically acceptable additives. In one aspect, the carrier is a liquid at 40° C. or more. In another aspect, the carrier is a liquid at 38° C. or more. In yet another aspect, the carrier is a liquid at 36° C. or more. In yet another aspect, the carrier is a liquid at 34° C. or more. In yet another aspect, the carrier is a liquid at 32° C. or more. In yet another aspect, the carrier is a liquid at 30° C. or more. In yet another aspect, the carrier is a liquid at 27° C. or more. In yet another aspect, the carrier is a liquid at 24° C. or more. In yet another aspect, the carrier is a liquid at 20° C. or more. In yet another aspect, the carrier is a liquid at 16° C. or more. In yet another aspect, the carrier is a liquid at 12° C. or more. In one aspect of this embodiment, the active pharmaceutical ingredient has a solubility of greater than 5 mg/mL in the carrier at a temperature of 30-40° C. In one aspect of this embodiment, the active pharmaceutical ingredient has a solubility of greater than 25 mg/mL in the carrier at a temperature of 30-40° C. In one aspect of this embodiment, the active pharmaceutical ingredient has a solubility of greater than 50 mg/mL in the carrier at a temperature of 30-40° C. In one aspect of this embodiment, the active pharmaceutical ingredient has a solubility of greater than 75 mg/mL in the carrier at a temperature of 30-40° C. In one aspect of this embodiment, testosterone undecanoate has a solubility of greater than 100 mg/mL in the carrier at a temperature of 30-40° C. In one aspect of this embodiment, the active pharmaceutical ingredient has a solubility of greater than 130 mg/mL in the carrier at a temperature of 30-40° C. In one aspect of this embodiment, the active pharmaceutical ingredient has a solubility of greater than 160 mg/mL C in the carrier at a temperature of 30-40° C. In one aspect of this embodiment, the active pharmaceutical ingredient has a solubility of greater than 190 mg/mL in the carrier at a temperature of 30-40° C. In one aspect of this embodiment, the active pharmaceutical ingredient has a solubility of greater than 220 mg/mL in the carrier at a temperature of 30-40° C. In one aspect of this embodiment, the active pharmaceutical ingredient has a solubility of greater than 230 mg/mL in the carrier at a temperature of 30-40° C. In one aspect of this embodiment, the active pharmaceutical ingredient has a solubility of greater than 240 mg/mL in the carrier at a temperature 30-40° C. In one aspect of this embodiment, the active pharmaceutical ingredient has a solubility of greater than 250 mg/mL in the carrier at a temperature of 30-40° C. In one aspect of this embodiment, the active pharmaceutical ingredient has a solubility of greater than 260 mg/mL in the carrier at a temperature of 30-40° C. In one aspect of this embodiment, the active pharmaceutical ingredient has a solubility of greater than 270 mg/mL in the carrier at a temperature of 30-40° C. In one aspect of this embodiment, the active pharmaceutical ingredient has a solubility of greater than 280 mg/mL in the carrier at a temperature of 30-40° C. In one aspect of this embodiment, the active pharmaceutical ingredient has a solubility of greater than 290 mg/mL in the carrier at a temperature of 30-40° C. In one aspect of this embodiment, the active pharmaceutical ingredient has a solubility of greater than 300 mg/mL in the carrier at a temperature of 30-40° C. Typically, the solubility of the active pharmaceutical ingredient will be less than 600, 550, 500, 450, 400, 350 or 300 mg/mL. In one aspect of this embodiment, the carrier has 50% or less triglyceride. In one aspect of this embodiment, the carrier has 40% or less triglyceride. In one aspect of this embodiment, the carrier has 30% or less triglyceride. In one aspect of this embodiment, the carrier has 20% or less triglyceride. In one aspect of this embodiment, the carrier has 10% or less triglyceride. In one aspect of this embodiment, the carrier has 5% or less triglyceride. In one aspect of this embodiment, the carrier has 3% or less triglyceride. In one aspect of this embodiment, the carrier has 1% or less triglyceride. In one aspect of this embodiment, the carrier is substantially free of added triglyceride. In this context, substantially free of added triglyceride allows a level of triglyceride that is present as a minor or trace amount of another carrier that is used in the composition. For example, some monoglyceride carriers may also have an amount of triglyceride also as a component. Such a monoglyceride carrier if used in the composition described herein would be substantially free of added triglyceride. Conversely, if this same monoglyceride is used and a triglyceride is added to the carrier e.g., castor oil, this carrier is not considered substantially free of added triglyceride. In one aspect of this embodiment, one or more of the pharmaceutically acceptable carrier or excipients are (a) octadecanoic acid, (9Z)-octadec-9-enoic acid, hexadecanoic acid or (9Z,12Z)-9,12-Octadecadienoic acid (b) a mono-, di-, tri-propane-1,2,3-triol ester thereof, a combination of mono-, di-, or tri-propane-1,2,3-triol esters thereof or (c) any combination thereof. In one aspect, the pharmaceutically acceptable excipients or carriers can include polyethylene glycol, mono- or di-glyceride of stearic acid, palmitic acid, or a combination thereof, polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-poly(propylene oxide)-poly(ethylene oxide), polyvinyl alcohol, polystyrenesulfonic acid, polyvinylpyrrolidone-co-polyvinyl acetate, polyether polyol, carboxymethylcellulose, methylcellulose, hydroxyethyl cellulose, hydroxypropylmethyl cellulose phthalate, or hydroxypropylmethyl cellulose acetate succinate

In one embodiment, the pharmaceutical composition or dosage form can have an additive. According to this embodiment, the additive can be any pharmaceutically acceptable additive. In a specific aspect, the pharmaceutical acceptable additive has the function of providing the (17-β)-3-Oxoandrost-4-en-17-yl undecanoate in a form that provides sufficient bioavailability to a subject when administered orally. In one aspect, the additive allows for loading of the API in the carrier at levels above the solubility of the API in the carrier while not substantially compromising properties of the formulation. For example, in one aspect, the additive prevents or reduces or inhibits the amount of crystallization of the (17-β)-3-Oxoandrost-4-en-17-yl undecanoate. In one aspect, the additive increases the viscosity of the composition. In another aspect, the additive provides dissolution stability. In yet another aspect, the additive prevents or reduces degradation of (17-β)-3-Oxoandrost-4-en-17-yl undecanoate. In one aspect, the compositions have from about 20% to about 50% w/w, 23% to 35% w/w, or 26% to 32% w/w of API. In a specific aspect, the additive is a polyethylene glycol. In a specific aspect, the polyethylene glycol has an average molecular weight of less 1000. In another specific aspect, the polyethylene glycol has an average molecular weight of less than 800. In yet another specific aspect, the polyethylene glycol has an average molecular weight of less than 500. In one aspect, the polyethylene glycol has a melting point of less than 55° C. In one aspect, the polyethylene glycol has a melting point of less than 45° C. In one aspect, the polyethylene glycol has a melting point of less than 35° C. In one aspect, the polyethylene glycol has a melting point of less than 25° C. In one aspect, the polyethylene glycol has a melting point of less than 15° C. In one aspect, the polyethylene glycol has a melting point of less than 10° C. In another aspect, the additive is a mono- or di-glyceride of stearic acid, palmitic acid, or a combination thereof. In one aspect, the additive is polvinylpyrrolidone. In one aspect, the additive is hydroxypropyl methylcellulose. In one aspect, the additive is hydroxypropyl cellulose. In one aspect, the additive is cellulose acetate phthalate. In one aspect, the additive is polyvinyl acetate phthalate. In one aspect, the additive is polyethylene oxide. In one aspect, the additive is poly(acrylic acid). In one aspect, the additive is polymethyacrylate. In one aspect, the additive is poly(ethylene oxide)-poly(propylene oxide)-poly(ethylene oxide). In one aspect, the additive is polyvinyl alcohol. In one aspect, the additive is polystyrenesulfonic acid. In one aspect, the additive is polyvinylpyrrolidone-co-polyvinyl acetate. In one aspect, the additive is polyether polyol. In one aspect, the additive is carboxymethylcellulose. In one aspect, the additive is methylcellulose. In one aspect, the additive is hydroxyethyl cellulose. In one aspect, the additive is hydroxypropylmethyl cellulose phthalate. In one aspect, the additive is hydroxypropylmethyl cellulose acetate succinate.

Exemplary Hard Gel Capsule Formulations

Provided in this section are exemplary hard gel capsule fill formulations having from about 20% to about 50% w/w, 23% to 35% w/w, or 26% to 32% w/w, an active pharmaceutical ingredient which is (17-β)-3-Oxoandrost-4-en-17-yl undecanoate and a pharmaceutically acceptable carrier which is a carrier for the active pharmaceutical ingredient. Typically, these formulations are non-liquid at room temperature. More specifically, the carrier is a solvent for the active pharmaceutical ingredient. Desirably, the carrier allows for high concentrations of the active pharmaceutical ingredient without substantially compromising bioavailability. In some specific aspects, the carrier also includes one or more pharmaceutically acceptable additives (e.g., stabilizing agent). In one aspect, the carrier is a liquid at 40° C. or more. In another aspect, the carrier is a liquid at 38° C. or more. In yet another aspect, the carrier is a liquid at 36° C. or more. In yet another aspect, the carrier is a liquid at 34° C. or more. In yet another aspect, the carrier is a liquid at 32° C. or more. In yet another aspect, the carrier is a liquid at 30° C. or more. In yet another aspect, the carrier is a liquid at 27° C. or more. In yet another aspect, the carrier is a liquid at 24° C. or more. In yet another aspect, the carrier is a liquid at 20° C. or more. In yet another aspect, the carrier is a liquid at 16° C. or more. In yet another aspect, the carrier is a liquid at 12° C. or more. In one aspect of this embodiment, the active pharmaceutical ingredient has a solubility of greater than 5 mg/mL in the carrier at a temperature of 30-40° C. In one aspect of this embodiment, the active pharmaceutical ingredient has a solubility of greater than 25 mg/mL in the carrier at a temperature of 30-40° C. In one aspect of this embodiment, the active pharmaceutical ingredient has a solubility of greater than 50 mg/mL in the carrier at a temperature of 30-40° C. In one aspect of this embodiment, the active pharmaceutical ingredient has a solubility of greater than 75 mg/mL in the carrier at a temperature of 30-40° C. In one aspect of this embodiment, testosterone undecanoate has a solubility of greater than 100 mg/mL in the carrier at a temperature of 30-40° C. In one aspect of this embodiment, the active pharmaceutical ingredient has a solubility of greater than 130 mg/mL in the carrier at a temperature of 30-40° C. In one aspect of this embodiment, the active pharmaceutical ingredient has a solubility of greater than 160 mg/mL C in the carrier at a temperature of 30-40° C. In one aspect of this embodiment, the active pharmaceutical ingredient has a solubility of greater than 190 mg/mL in the carrier at a temperature of 30-40° C. In one aspect of this embodiment, the active pharmaceutical ingredient has a solubility of greater than 220 mg/mL in the carrier at a temperature of 30-40° C. In one aspect of this embodiment, the active pharmaceutical ingredient has a solubility of greater than 230 mg/mL in the carrier at a temperature of 30-40° C. In one aspect of this embodiment, the active pharmaceutical ingredient has a solubility of greater than 240 mg/mL in the carrier at a temperature 30-40° C. In one aspect of this embodiment, the active pharmaceutical ingredient has a solubility of greater than 250 mg/mL in the carrier at a temperature of 30-40° C. In one aspect of this embodiment, the active pharmaceutical ingredient has a solubility of greater than 260 mg/mL in the carrier at a temperature of 30-40° C. In one aspect of this embodiment, the active pharmaceutical ingredient has a solubility of greater than 270 mg/mL in the carrier at a temperature of 30-40° C. In one aspect of this embodiment, the active pharmaceutical ingredient has a solubility of greater than 280 mg/mL in the carrier at a temperature of 30-40° C. In one aspect of this embodiment, the active pharmaceutical ingredient has a solubility of greater than 290 mg/mL in the carrier at a temperature of 30-40° C. In one aspect of this embodiment, the active pharmaceutical ingredient has a solubility of greater than 300 mg/mL in the carrier at a temperature of 30-40° C. Typically, the solubility of the active pharmaceutical ingredient will be less than 600 mg/mL. In one aspect of this embodiment, the carrier has 50% w/w or less triglyceride. In one aspect of this embodiment, the carrier has 40% w/w or less triglyceride. In one aspect of this embodiment, the carrier has 30% w/w or less triglyceride. In one aspect of this embodiment, the carrier has 20% w/w or less triglyceride. In one aspect of this embodiment, the carrier has 10% w/w or less triglyceride. In one aspect of this embodiment, the carrier has 5% w/w or less triglyceride. In one aspect of this embodiment, the carrier has 3% or less triglyceride. In one aspect of this embodiment, the carrier has 1% w/w or less triglyceride. In one aspect of this embodiment, the carrier is substantially free of added triglyceride. In this context, substantially free of added triglyceride allows a level of triglyceride that is present as a minor or trace amount of another carrier that is used in the composition. For example, some monoglyceride carriers may also have an amount of triglyceride also as a component. Such a monoglyceride carrier if used in the composition described herein would be substantially free of added triglyceride. Conversely, if this same monoglyceride is used and a triglyceride is added to the carrier e.g., castor oil, this carrier is not considered substantially free of added triglyceride. In one aspect of this embodiment, one or more of the pharmaceutically acceptable carrier or excipients are (a) octadecanoic acid, (9Z)-octadec-9-enoic acid or hexadecanoic acid (b) a mono-, di-, tri-propane-1,2,3-triol ester thereof, a combination of mono-, di-, or tri-propane-1,2,3-triol esters thereof or (c) any combination thereof. In one aspect, the pharmaceutically acceptable excipients or carriers can include polyethylene glycol, mono- or di-glyceride of stearic acid, palmitic acid, or a combination thereof, polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-poly(propylene oxide)-poly(ethylene oxide), polyvinyl alcohol, polystyrenesulfonic acid, polyvinylpyrrolidone-co-polyvinyl acetate, polyether polyol., carboxymethylcellulose, methylcellulose, hydroxyethyl cellulose, hydroxypropylmethyl cellulose phthalate, or hydroxypropylmethyl cellulose acetate succinate.

The composition or dosage form as described herein can optionally include any of the following additives (some of which as the skilled artisan recognizes will act as the stabilizing agent or additive that allows for the production of compositions having the beneficial properties described herein) in the following seven paragraphs as long as the properties of the composition or dosage form are consistent with those as described herein.

Suitable additives utilized in various embodiments described herein include, by way of non-limiting example, adsorbing agents, anti-adherents, anticoagulants, antifoaming agents, antioxidants, anti-caking agents, anti-static agents, binders, bile acids, bufferants, bulking agents, chelating agents, coagulants, colorants, co-solvent, opaquants, congealing agents, coolants, cryoprotectants, diluents, dehumidifying agents, desiccants, desensitizers, disintegrants, dispersing agents, enzyme inhibitors, glidants, fillers, hydrating agent, super disintegrants, gums, mucilages, hydrogen bonding agents, enzymes, flavorants, humectants, humidifying agents, lubricant oils, ion-exchange resins, lubricants, plasticizers, pH modifying agents, preservatives, solidifying agent, solvents, solubilizers, spreading agent sweeteners, stabilizers, surface area enhancing agents, suspending agent, thickeners, viscosity increasing agents, waxes and mixtures thereof

Some non-limiting examples of the additives suitable for the present disclosure may be: alcohols and/or polyols (e.g. ethanol, isopropanol, butanol, benzyl alcohol, ethylene glycol, propylene glycol, glycerol, sorbitol, mannitol, dimethyl isosorbide, polyethylene glycol, fatty acid alcohol, vinyl alcohol polypropylene glycol, polyvinylalcohol, tocopherols, cellulose cyclodextrins, other derivatives, forms, mixtures thereof, or the like); ethers of polyethylene glycols having an average molecular weight of about 200 to about 20,000 (e.g. tetrahydrofurfuryl alcohol PEG ether, methoxy PEG, or the like); amides (e.g. 2-pyrrolidone, 2-piperidone, 8-caprolactam, N-alkylpyrrolidone, N-hydroxyalkylpyrrolidone, N-alkylpiperidone, N-alkylcaprolactam, dimethylacetamide, polyvinylpyrrolidone and the like.); esters (e.g. ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, 8-caprolactone and isomers thereof, 6-valerolactone and isomers thereof, gamma-butyrolactone and isomers thereof; and other additives known in the art, such as dimethyl acetamide, dimethyl isosorbide, N-methylpyrrolidones, monooctanoin, diethylene glycol monoethyl ether, or the like); amino acids (e.g. p-aminobenzamidine, sodium glycocholate) mesylate; amino acids and modified amino acids (e.g. aminoboronic acid derivatives and n-acetylcysteine; peptides and modified peptides (e.g. bacitracin, phosphinic acid dipeptide derivatives, pepstatin, antipain, leupeptin, chymostatin, elastin, bestatin, phoshporamindon, puromycin, cytochalasin potatocarboxy peptidase inhibitor, amastatin, or the like); polypeptide protease inhibitors; mucoadhesive polymers (e.g. polyacrylate derivatives, chitosan, cellulosics, chitosan-EDTA, chitosan-EDTA-antipain, polyacrylic acid, carboxymethyl cellulose etc.) or the like; or combinations thereof.

Some more examples of suitable additives for compositions and/or dosage forms described herein include, by way of non-limiting example, talc, magnesium stearate, silica (e.g. fumed silica, micronized silica, magnesium aluminum silicate etc.) and/or derivatives, polyethylene glycols, surfactants, waxes, oils, cetyl alcohol, polyvinyl alcohol, stearic acid, stearic acid salts, stearic acid derivatives, starch, hydrogenated vegetable oils, hydrogenated castor oils, sodium benzoate, sodium acetate, leucine, PEG, alkyl sulfate salts; acetylated monoglycerides; long-chain alcohols; silicone derivatives; butylated hydroxy toluene (BHT), butylated hydroxyl anisole (BHA), gallic acid, propyl gallate, ascorbic acid, ascorbyl palmitate, 4-hydroxymethyl-2,6-di-tert-butyl phenol, dry starch, dry sugars, polyvinyl pyrrolidones, starch paste, methacrylic copolymers, bentonite, sucrose, polymeric cellulose derivatives, shellac, sugar syrup; corn syrup; polysaccharides, acacia, tragacanth, guar gum, xanthan gums; alginates; gelatin; gelatin hydrolysate; agar; sucrose; dextrose; PEG, vinyl pyrrolidone copolymers, poloxamers; pregelatinized starch, sorbitol, glucose); acetic acid, hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, vinegar, pharmaceutically acceptable bases, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamin; salt of a pharmaceutically acceptable cation and an anion; EDTA and EDTA salts; titanium dioxide, food dyes, lakes, natural vegetable colorants, iron oxides, silicates, sulfates, magnesium hydroxide and aluminum hydroxide; halogenated hydrocarbons, trichloroethane, trichloroethylene, dichloromethane, fluorotrichloromethane, diethylether, trehalose, phosphates, citric acid, tartaric acid, gelatin, dextran and mannitol, lactose, mannitol, sodium chloride, potassium chloride, spray-dried lactose, hydrolyzed starches, directly compressible starch, microcrystalline cellulose, cellulosic derivatives, sorbitol, sucrose, sucrose-based materials, calcium sulfate, dibasic calcium phosphate, dextrose, croscarmellose sodium, starch, starch derivatives, clays, gums, cellulose, cellulose derivatives, alginates, crosslinked polyvinylpyrrolidone, sodium starch glycolate and microcrystalline cellulose, magnesium oxide, magnesium carbonates; desensitizers, spray-dried flavors, essential oils, ethyl vanillin, styrene/divinyl benzene copolymers, quaternary ammonium compounds, polyethylene glycol, citrate esters (such as triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl sebacate, ascorbic acid, boric acid, sorbic acid, benzoic acid, and salts thereof, parabens, phenols, benzyl alcohol, and quaternary ammonium compounds; alcohols, ketones, esters, chlorinated hydrocarbons water; sweeteners (e.g. maltose, sucrose, glucose, sorbitol, glycerin and dextrins, aspartame, saccharine, saccharine salts, glycyrrhizin), viscosity modifiers, sugars, polyvinylpyrrolidone, cellulosics, polymers, gums and/or alginates.

In one embodiment, additives may also be materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum Arabic); spermaceti; natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based polymers (e.g., ethyl cellulose, methyl cellulose, microcrystalline cellulose, sodium carboxymethyl cellulose, hydroxypropylmethyl cellulose, ethyl cellulose, hydroxypropyl cellulose, HPMC acid succinates, cellulose acetate, cellulose nitrate, cellulose acetate butyrate, cellulose acetate trimellitate, carboxymethylethyl cellulose, hydroxypropylmethyl cellulose phthalate), shellacs; inorganics, such as dicalcium phosphate, hydroxyapatite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar. Non-limiting examples of compounds (e.g., additives) that can be used as at least a part of the pharmaceutically acceptable carrier include without limitation celluloses; dextrins, gums, carbomers, methacrylates, sugars, lactoses, inorganic carbonates, oxides, chlorides, sulphates and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

In one embodiment, the additive may include at least one component selected from celluloses, dextrins, gums, carbomers, methacrylates, inorganic carbonates, salts of calcium, salts of magnesium, fatty acids, fatty acid esters, gelatin, lactoses, polyethylene glycol, polyethylene oxide co-polymers, silicates, partially hydrogenated vegetable oils, fully hydrogenated vegetable oils, waxes, antioxidants, tocopherol, sugar stearates, starches, shellac, resins, proteins, and combinations thereof.

In another embodiment, the additive may include at least one component selected from celluloses, dextrins, gums, carbomers, methacrylates, sugars, lactoses, inorganic carbonates, salts of calcium, salts of magnesium, salts of fatty acids, inorganic and organic acids, bases and salts, propylene glycol, glycerols, fatty acids, fatty alcohols, fatty acid esters, glycerol esters, mono-glycerol esters of fatty acids, di-glycerol esters of fatty acids, mixtures of mono-glycerol and di-gylcerol esters of fatty acids, omega oils, waxes, alcohols, gelatin, polyethylene glycol, polyethylene oxide co-polymers, silicates, antioxidants, tocopherol, sugar stearates, starches, shellac, resins, proteins, acrylates, methyl copolymers, polyvinyl alcohol, starch, phthalates, and combinations thereof.

Non-limiting examples of additives as release modulators that may be used include lipophilic resins; ethyl cellulose (EC), methylethyl cellulose (MEC), carboxymethyl ethylcellulose (CMEC), hydroxyethyl cellulose (HEC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose phthalate (HPMCP), hydroxypropyl methyl cellulose acetate succinate (HPMCAS), hydroxypropyl methyl cellulose acetate trimellitate (HPMCAT), ion-exchange resin; poloxamers; and ethylhydroxy ethylcellulose (EHEC) tocopherol; shellac; and combinations thereof. Non-limiting examples of lipidic lipophilic release modulators include fatty acids; mono-, di-, tri-esters of fatty acids with glycerol; sucrose esters with fatty acids; cetyl alcohol; stearic acid; glyceryl monostearate; glyceryl distearate; glyceryl tristearate; glyceryl palmitostearate; hydrogenated castor oil; butyl and glycol esters of fatty acids; oleic acid; cetyl alcohol; stearyl alcohol; cetostearyl alcohol; hydrogenated vegetable oil; waxes; bees wax; lard; omega fatty acid esters; hydrogenated soybean oil; hydrogenated vegetable oil; hydrogenated cottonseed and castor oil; partially hydrogenated soybean oil; partially hydrogenated castor oil; partially soy and cottonseed oil; phospholipids; hydrogenated oils, and their derivatives and combinations thereof.

Exemplary Formulations Embodiments

Provided in this section are formulations.

TABLE 1
Drug + Carriers

| | Compositions (w/w %) | | Ratio of API: Carrier in |
|---|---|---|---|
| | Testosterone | | a pharmaceutical |
| Composition | undecanoate* | Carrier | composition |
| A | 10-15 | 85-90 | 1:5.7-1:9.0 |
| B | 15-20 | 80-85 | 1:4.0-1:5.7 |
| C | 20-30 | 70-80 | 1:2.3-1:4.0 |
| D | 30-40 | 60-70 | 1:1.5-1:2.3 |
| E | 40-50 | 50-60 | 1:1.0-1:1.5 |

*As an active ingredient, it can be untreated, sieved (PS <450 micron), milled (PS <150 micron), micronized (1 micron < PS < 25 micron), or nanosized (PS <1 micron).

TABLE 2
Carrier Components

| | | Carrier No. | | | | | | | | | | | | |
|---|---|---|---|---|---|---|---|---|---|---|---|---|---|---|
| Component | | I | II | III | IV | V | VI | VII | VIII | IX | X | XI | XII | XIII |
| Solubilizer | Propylene | Y | — | — | — | — | — | — | — | — | — | — | — | — |
| | glycol mono | | | | | | | | | | | | | |
| | or di-laurate | | | | | | | | | | | | | |
| | Propylene | — | Y | — | — | — | — | — | — | — | — | — | — | — |
| | glycol mono | | | | | | | | | | | | | |
| | or di-caprylate | | | | | | | | | | | | | |
| | Corn | — | — | Y | — | — | — | — | — | — | — | — | — | — |
| | glycerides | | | | | | | | | | | | | |
| | (e.g. Glyceryl | | | | | | | | | | | | | |
| | mono or di- | | | | | | | | | | | | | |
| | linoleate) | | | | | | | | | | | | | |
| | Vegetable | — | — | — | Y | — | — | — | — | — | — | — | — | — |
| | glycerides | | | | | | | | | | | | | |
| | (e.g. Glyceryl | | | | | | | | | | | | | |
| | mono or di- | | | | | | | | | | | | | |
| | oleate) | | | | | | | | | | | | | |
| | Glyceryl mono | — | — | — | — | Y | — | — | — | — | — | — | — | — |
| | or di-stearate | | | | | | | | | | | | | |
| | Glyceryl | | | | | | | | | | | | | |
| | palmito- | — | — | — | — | — | Y | — | — | — | — | — | — | — |
| | stearate | | | | | | | | | | | | | |
| | (9Z)-Octadec- | — | — | — | — | — | — | Y | — | — | — | — | — | — |
| | 9-enoic acid | | | | | | | | | | | | | |
| | Octadecanoic | — | — | — | — | — | — | — | Y | — | — | — | — | — |
| | acid | | | | | | | | | | | | | |
| | (9Z,12Z)-9,12- | — | — | — | — | — | — | — | — | Y | — | — | — | — |
| | Octa- | | | | | | | | | | | | | |
| | decadienoic | | | | | | | | | | | | | |
| | acid | | | | | | | | | | | | | |
| | Peppermint oil | — | — | — | — | — | — | — | — | — | Y | — | — | — |
| | Omega-3 | — | — | — | — | — | — | — | — | — | — | Y | — | — |
| | EPA/DHA | | | | | | | | | | | | | |
| | Vitamin E | — | — | — | — | — | — | — | — | — | — | — | Y | — |
| | Combinations | — | — | — | — | — | — | — | — | — | — | — | — | Y |
| | * | | | | | | | | | | | | | |
| Hydro- | Cremophor, | Y | Y | Y | Y | Y | Y | Y | Y | Y | Y | Y | Y | Y |
| philic | Tween, SLS, | | | | | | | | | | | | | |
| Add. | Poloxamer, | | | | | | | | | | | | | |
| | Polymer, | | | | | | | | | | | | | |
| | and/or | | | | | | | | | | | | | |
| | combinations | | | | | | | | | | | | | |
| Other | Anti-oxidant, | q.s. | q.s. | q.s. | q.s. | q.s. | q.s. | q.s. | q.s. | q.s. | q.s. | q.s. | q.s. | q.s. |
| Add. | solidifier, flow | | | | | | | | | | | | | |
| | agent, solvent, | | | | | | | | | | | | | |
| | and/or | | | | | | | | | | | | | |
| | combinations | | | | | | | | | | | | | |

* Combinations of solubilizers can be a combination of 2 or more solubilizers that are listed in this table as well as include propylene glycol, polyethylene glycol, glycerol, sorbitol, DMA, and so on.
Add. = additive.
Y = Yes.

Carrier I. Compositions composed of solubilizer (Propylene glycol mono or di-laurate),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | % Release in |
| | | Cremophor² RH | | | | | 8% Triton |
| | | 40, Tween³ 80, | | | | | Aqueous |
| | Propylene | SLS, Poloxamer⁴ | | | | | Media |
| | glycol | 407, Polymer⁵, | Other additives⁷ | | | | ≥50% in 2 hrs |
| Comp. | mono or | and/or | Anti- | Solidify | | Total % | and ≥75% in |
| I No. | di-laurate | combinations⁶) | oxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| | | 30-40 | 0-1 | 0-10 | 0-10 | 100 | Yes |
| g | 50-80 | 20-30 | 0-1 | 0-10 | 0-10 | 100 | Yes |
| | | 10-20 | 0-1 | 10-20 | 10-20 | 100 | No |

¹Hydrophilic additives can be, but are not limited to ones listed in this table, e.g., hydrophilic surfactants having an HLB value of greater than 10, which are PEG-8 caprylic/capric glycerides (Labrasol), lauroyl macrogol-32 glyceride (Gelucire 44/14), stearoyl macrogol glyceride (Gelucire 50/13), sodium dioctyl sulfosuccinate, polyethylene glycol fatty acids mono- and di- ester mixtures, polyethylene glycol 1000 tocopherol succinate, phytosterols, phytosterol fatty acid esters, lanosterol PEG-24 cholesterol ether, PEG-30 soya sterol, PEG-25 phytosterol, PEG-30 cholestanol, and so on.
2Cremophor includes, but is not limited to, Cremophor RH 40, but Cremophor EL, RH 40, and RH 60.
³Tween includes, but is not limited to, Tween 80, but Tween 20, 60, and 80.
⁴Poloxamer includes, but is not limited to Poloxamer 407, but Poloxamer 124, 188, 234, 335, and 407.
⁵Polymer includes, but is not limited to, Polyethylene glycol, Hydroxypropyl cellulose, Hydroxypropylmethyl cellulose, Hydroxypropylmethyl cellulose acetate succinate, Polyvinylpyrrolidone, Polyvinyl acetate, Polylactic-co-glycolic acid, Polyvinyl caprolactame, Carbomer, and a combination thereof
⁶Combinations of hydrophilic additives can be 2 or more hydrophilic additives.
⁷Other additives can be, but are not limited to ones listed in this table, e.g., adsorbing agents, anti-adherents, anticoagulants, antifoaming agents, anti-caking agents, anti-static agents, binders, bile acids, bufferants, bulking agents, chelating agents, coagulants, colorants, opaquants, coolants, cryoprotectants, diluents, dehumidifying agents, desiccants, desensitizers, disintegrants, dispersing agents, enzyme inhibitors, fillers, hydrating agent, super disintegrants, gums, mucilages, hydrogen bonding agents, enzymes, flavorants, humectants, humidifying agents, ion-exchange resins, lubricants, plasticizers, pH modifying agents, preservatives, organic solvents, spreading agent, stabilizers, suspending agent, thickeners, viscosity increasing agents, waxes, and so on.
⁸Anti-oxidant can be, but is not limited to, ascorbyl palmitate, ascorbic acid, butylated hydroxyanisole (BHA), butylated hydroxytoluene (BHT), propyl gallate, cysteine, sodium metabisulfite (SMB), thiol derivatives, alpha-tocopherol, and so on.
⁹Solidifying (solidify) agent can be, but is not limited, to PEG 3350, PEG 4000, PEG 6000, PEG 8000, Poloxamer 188, Poloxamer 407, cetyl esters, wax, beeswax, glyceryl monostearate, glyceryl distearate, glyceryl palmitostearate, stearic acid, and so on.
¹⁰Combinations of other additives can be 2 or more other additives.

Descriptions for from ¹ to ¹⁰ are applied to tables of all carrier compositions (from Carrier I to Carrier XIII tables) shown below.

Carrier II. Compositions composed of solubilizer (Propylene glycol mono or di-caprylate),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | % Release in |
| | | Cremophor² RH | | | | | 8% Triton |
| | Propylene | 40, Tween³ 80, | | | | | Aqueous |
| | glycol | SLS, Poloxamer⁴ | | | | | Media |
| | mono or | 407, Polymer⁵, | Other additives⁷ | | | | ≥50% in 2 hrs |
| Comp. | di- | and/or | Anti- | Solidify | | Total % | and ≥75% in |
| II No. | caprylate | combinations⁶) | oxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier III. Compositions composed of solubilizer (Corn glycerides: e.g. Glyceryl mono
or di-linoleate), hydrophilic additives, and other additives for Composition A to E

| | Carrier Compositions (w/w %) | | | | | | |
|---|---|---|---|---|---|---|---|
| | | Hydrophilic | | | | | |
| | Corn | additives¹ (e.g. | | | | | % Release in |
| | glycerides | Cremophor² RH | | | | | 8% Triton |
| | (e.g. | 40, Tween³ 80, | | | | | Aqueous |
| | Glyceryl | SLS, Poloxamer⁴ | | | | | Media |
| | mono or | 407, Polymer⁵, | Other additives⁷ | | | | ≥50% in 2 hrs |
| Comp. | di- | and/or | Anti- | Solidify | | Total % | and ≥75% in |
| III No. | linoleate) | combinations⁶) | oxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier IV. Compositions composed of solubilizer (Vegetable glycerides: e.g. Glyceryl
mono or di-oleate), hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | % Release in |
| | Vegetable | Cremophor² RH | | | | | 8% Triton |
| | glycerides | 40, Tween³ 80, | | | | | Aqueous |
| | (e.g. | SLS, Poloxamer⁴ | | | | | Media |
| | Glyceryl | 407, Polymer⁵, | Other additives⁷ | | | | ≥50% in 2 hrs |
| Comp. | mono or | and/or | Anti- | Solidify | | Total % | and ≥75% in |
| IV No. | di-oleate) | combinations⁶) | oxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier V. Compositions composed of solubilizer (Glyceryl mono or di-stearate),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | % Release in |
| | | Cremophor² RH | | | | | 8% Triton |
| | | 40, Tween³ 80, | | | | | Aqueous |
| | | SLS, Poloxamer⁴ | | | | | Media |
| | Glyceryl | 407, Polymer⁵, | Other additives⁷ | | | | ≥50% in 2 hrs |
| Comp. | mono or | and/or | Anti- | Solidify | | Total % | and ≥75% in |
| V No. | di-stearate | combinations⁶) | oxidant⁸ | agent⁹ | Combinations¹⁰ | in Carrier | 4 hrs |
| a | 90-100 | — | 0-1 | 0-10 | 0-10 | 100 | No |
| b | 45-99 | 1-40 | 0-1 | 0-15 | 0-15 | 100 | No |
| | | 1-25 | 0-1 | 15-20 | 15-20 | 100 | No |
| c | 85-99 | 1-4.5 | 0-1 | 0-10 | 0-10 | 100 | No |
| d | 80-95 | 5-10 | 0-1 | 0-10 | 0-10 | 100 | No |
| e | 70-90 | 10-20 | 0-1 | 0-10 | 0-10 | 100 | No |
| f | 60-80 | 20-30 | 0-1 | 0-10 | 0-10 | 100 | No |
| | | 10-20 | 0-1 | 10-20 | 10-20 | 100 | No |
| g | 50-80 | 30-40 | 0-1 | 0-10 | 0-10 | 100 | No |
| | | 20-30 | 0-1 | 0-10 | 0-10 | 100 | No |
| | | 10-20 | 0-1 | 10-20 | 10-20 | 100 | No |

Carrier VI. Compositions composed of solubilizer (Glyceryl palmito-stearate),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | | 40, Tween³ 80, | | | | | 8% Triton |
| | | SLS, Poloxamer⁴ | | | | | Aqueous Media |
| | Glyceryl | 407, Polymer⁵, | Other additives⁷ | | | | ≥50% in 2 hrs |
| Comp. | palmito- | and/or | Anti- | Solidify | Combi- | Total % | and ≥75% in |
| VI No. | stearate | combinations⁶) | oxidant⁸ | agent⁹ | nations¹⁰ | in Carrier | 4 hrs |
| a | 90-100 | — | 0-1 | 0-10 | 0-10 | 100 | No |
| b | 45-99 | 1-40 | 0-1 | 0-15 | 0-15 | 100 | No |
| | | 1-25 | 0-1 | 15-20 | 15-20 | 100 | No |
| c | 85-99 | 1-4.5 | 0-1 | 0-10 | 0-10 | 100 | No |
| d | 80-95 | 5-10 | 0-1 | 0-10 | 0-10 | 100 | No |
| e | 70-90 | 10-20 | 0-1 | 0-10 | 0-10 | 100 | No |
| f | 60-80 | 20-30 | 0-1 | 0-10 | 0-10 | 100 | No |
| | | 10-20 | 0-1 | 10-20 | 10-20 | 100 | No |
| g | 50-80 | 30-40 | 0-1 | 0-10 | 0-10 | 100 | No |
| | | 20-30 | 0-1 | 0-10 | 0-10 | 100 | No |
| | | 10-20 | 0-1 | 10-20 | 10-20 | 100 | No |

Carrier VII. Compositions composed of solubilizer ((9Z)-Octadec-9-enoic
acid), hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | | 40, Tween³ 80, | | | | | 8% Triton |
| | (9Z)- | SLS, Poloxamer⁴ | | | | | Aqueous Media |
| | Octadec- | 407, Polymer⁵, | Other additives⁷ | | | | ≥50% in 2 hrs |
| Comp. | 9-enoic | and/or | Anti- | Solidify | Combi- | Total % | and ≥75% in |
| VII No. | acid | combinations⁶) | oxidant⁸ | agent⁹ | nations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier VIII. Compositions composed of solubilizer (octadecanoic acid),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | | 40, Tween³ 80, | | | | | 8% Triton |
| | | SLS, Poloxamer⁴ | | | | | Aqueous Media |
| | Octa- | 407, Polymer⁵, | Other additives⁷ | | | | ≥50% in 2 hrs |
| Comp. | decanoic | and/or | Anti- | Solidify | Combi- | Total % | and ≥75% in |
| VIII No. | acid | combinations⁶) | oxidant⁸ | agent⁹ | nations¹⁰ | in Carrier | 4 hrs |
| a | 90-100 | — | 0-1 | 0-10 | 0-10 | 100 | No |
| b | 45-99 | 1-40 | 0-1 | 0-15 | 0-15 | 100 | No |
| | | 1-25 | 0-1 | 15-20 | 15-20 | 100 | No |
| c | 85-99 | 1-4.5 | 0-1 | 0-10 | 0-10 | 100 | No |
| d | 80-95 | 5-10 | 0-1 | 0-10 | 0-10 | 100 | No |
| e | 70-90 | 10-20 | 0-1 | 0-10 | 0-10 | 100 | No |
| f | 60-80 | 20-30 | 0-1 | 0-10 | 0-10 | 100 | No |
| | | 10-20 | 0-1 | 10-20 | 10-20 | 100 | No |
| g | 50-80 | 30-40 | 0-1 | 0-10 | 0-10 | 100 | No |
| | | 20-30 | 0-1 | 0-10 | 0-10 | 100 | No |
| | | 10-20 | 0-1 | 10-20 | 10-20 | 100 | No |

Carrier IX. Compositions composed of solubilizer ((9Z,12Z)-9,12-Octadecadienoic
acid), hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | (9Z,12Z)- | 40, Tween³ 80, | | | | | 8% Triton |
| | 9,12- | SLS, Poloxamer⁴ | | | | | Aqueous Media |
| | Octadeca- | 407, Polymer⁵, | Other additives⁷ | | | | ≥50% in 2 hrs |
| Comp. | dienoic | and/or | Anti- | Solidify | Combi- | Total % | and ≥75% in |
| IX No. | acid | combinations⁶) | oxidant⁸ | agent⁹ | nations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier X. Compositions composed of solubilizer (Peppermint oil),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | | 40, Tween³ 80, | | | | | 8% Triton |
| | | SLS, Poloxamer⁴ | | | | | Aqueous Media |
| | | 407, Polymer⁵, | Other additives⁷ | | | | ≥50% in 2 hrs |
| Comp. | Peppermint | and/or | Anti- | Solidify | Combi- | Total % | and ≥75% in |
| X No. | oil | combinations⁶) | oxidant⁸ | agent⁹ | nations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | Yes |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | Yes |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | Yes |

Carrier XI. Compositions composed of solubilizer (Omega-3 EPA/DHA),
hydrophilic additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | | 40, Tween³ 80, | | | | | 8% Triton |
| | | SLS, Poloxamer⁴ | | | | | Aqueous Media |
| | | 407, Polymer⁵, | Other additives⁷ | | | | ≥50% in 2 hrs |
| Comp. | Omega-3 | and/or | Anti- | Solidify | Combi- | Total % | and ≥75% in |
| XI No. | EPA/DHA | combinations⁶) | oxidant⁸ | agent⁹ | nations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier XII. Compositions composed of solubilizer (Vitamin E), hydrophilic
additives, and other additives for Composition A to E

| | Carrier Compositions (w/w %) | | | | | | |
|---|---|---|---|---|---|---|---|
| | | Hydrophilic | | | | | |
| | | additives¹ (e.g. | | | | | |
| | | Cremophor² RH | | | | | % Release in |
| | | 40, Tween³ 80, | | | | | 8% Triton |
| | | SLS, Poloxamer⁴ | | | | | Aqueous Media |
| | | 407, Polymer⁵, | Other additives⁷ | | | | ≥50% in 2 hrs |
| Comp. | | and/or | Anti- | Solidify | Combi- | Total % | and ≥75% in |
| XII No. | Vitamin E | combinations⁶) | oxidant⁸ | agent⁹ | nations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | Yes |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | Yes |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | Yes |

Carrier XIII. Compositions composed of a combination of solubilizers,
hydrophilic additives, and other additives for Composition A to E

| | Carrier Compositions (w/w %) | | | | | | |
|---|---|---|---|---|---|---|---|
| | Combination of | Hydrophilic | | | | | |
| | solubilizers^{†} | additives¹ (e.g. | | | | | % Release in |
| | (e.g. oleic acid | Cremophor² RH 40, | | | | | 8% Triton |
| | and GDS, oleic | Tween³ 80, SLS, | | | | | Aqueous Media |
| | acid and pepper- | Poloxamer⁴ 407, | Other additives⁷ | | | | ≥50% in 2 hrs |
| Comp. | mint oil, maisine | Polymer⁵, and/or | Anti- | Solidifying | Combi- | Total % | and ≥75% in |
| XIII No. | 35-1 and GMC, etc) | combinations⁶) | oxidant⁸ | agent⁹ | nations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

^{†}Combination of solubilizers includes, not limited to oleic acid and glyceryl distearate, oleic acid and peppermint oil, or maisine 35-1 and glyceryl monocaprylate, but 2 or more solubilizers that are listed in Table 2.

In one aspect, the composition of this embodiment in the Tables above releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c) when tested using USP type 2 apparatus in about e.g., 1000 mL 8% Triton X100 solution in water at a specific temperature (e.g., 37° C.) at 100 rpm. In one aspect, the composition of this embodiment releases (a) at least 75% or more at 2 hours and (b) less than 95% or 90% at 0.25 hours.

Methods of Use

Subjects that can be treated by pharmaceutical compositions and unit dosage forms of the present disclosure (e.g., prepared from or comprising substantially pure (17-β)-3-Oxoandrost-4-en-17-yl undecanoate substantially free of impurities) can be any mammal (e.g., male or female) in need thereof In particular, in one embodiment, the human male is at least 14, 16, or 18 years of age.

In another embodiment, the human male is at least age 20, 21, 25 or 30. In a further embodiment, the subject is an adult male of at least age 40 or 50. In yet a further embodiment, the subject can be an adult male of at least age 60. Subjects that can be treated by pharmaceutical compositions and unit dosage forms of the present disclosure (e.g., prepared from or comprising substantially pure (17-β)-3-Oxoandrost-4-en-17-yl undecanoate substantially free of impurities) can be any human male in need thereof. In particular, in one embodiment, the he man female may be at least 14 years of age. In some embodiments, the human subject is a female. For example, in another embodiment, the human female is an adult of at least 20 or 30 years of age. In a further embodiment, the subject can be an adult female of at least age 40 or 50. In a further embodiment, the subject can be an adult female who is deficient in the endogenous serum testosterone levels. In a. further embodiment, the subject can be an adult female who has undergone unilateral or bilateral oophorectomy. In yet a further embodiment, the subject can be an adult female who has undergone unilateral or bilateral oophorectomy. In yet another embodiment, the subject can be a post-menopausal woman.

As discussed above, a method of treating a human subject in need of testosterone therapy is provided. The method can include the steps of administering any of the pharmaceutical to compositions or dosage forms (e.g., capsule or tablet) disclosed herein. The pharmaceutical compositions and the dosage forms of the present invention can be used to treat any condition associated with testosterone deficiency, including complete absence, of endogenous testosterone in male or female subjects. Examples of conditions associated with testosterone deficiency that can be treated using the dosage forms (e.g., capsule or tablet) or compositions described herein include, but are not limited to congenital or acquired primary hypogonadism, hypogonadotropic hypogonadism, cryptorchidism, bilateral torsion, orchitis, vanishing testis syndrome, orchidectomy, Klinefelter's syndrome, post castration, eunuchoidism, hypopituitarism, endocrine impotence, infertility due to spermatogenic disorders, impotence, male sexual dysfunction (MSD) including conditions such as premature ejaculation, erectile dysfunction, decreased libido, and the like, micropenis and constitutional delay, penile enlargement, appetite stimulation, testosterone deficiency associated with chemotherapy, testosterone deficiency associated with toxic damage from alcohol, testosterone deficiency associated with toxic damage from heavy metal, osteoporosis associated with androgen deficiency, or a combination thereof.

Other conditions that can be treated by the compositions and dosage forms disclosed herein include idiopathic gonadotropin, LHRH deficiency, or pituitary hypothalamic injury from tumors, trauma, or radiation. Typically, these subjects have low serum testosterone levels but have gonadotropins in the normal or low range. In one embodiment, the composition or oral dosage form can be used to stimulate puberty in carefully selected males with clearly delayed puberty not secondary to pathological disorder. In another embodiment, the composition or oral dosage form can be used in female-to-male transsexuals in order to maintain or restore male physical and sexual characteristics including body muscle mass, muscle tone, bone density, body mass index (BMI), enhanced energy, motivation and endurance, restoring psychosexual activity etc. In some embodiments, pharmaceutical composition or unit dosage form of the present disclosure (e.g., prepared from or comprising substantially pure (17-β)-3-Oxoandrost-4-en-17-yl undecanoate substantially free of impurities) can be useful in providing hormonal male contraception. In some embodiments, the pharmaceutical composition or unit dosage form of the present disclosure (e.g., prepared from or comprising substantially pure (17-β)-3-Oxoandrost-4-en-17-yl undecanoate substantially free of impurities) can be used to provide treatment of one or more symptoms associated with female sexual dysfunction, anorgasmia, osteoaithritis, hormonal male contraception. Additionally, the pharmaceutical composition or unit dosage form of the present disclosure (e.g., prepared from or comprising substantially pure (17-β)-3-Oxoandrost-4-en-17-yl undecanoate substantially free of impurities) can be used to treat and/or improve the patient related outcomes including the quality of life or wellbeing a subject suffering from deficiency of endogenous testosterone or who can otherwise benefit from the treatment. In some embodiments, the pharmaceutical composition or unit dosage form of the present disclosure (e.g., prepared from or comprising (17-β)-3-Oxoandrost-4-en-17-yl undecanoate substantially free of impurities) can be used to treat or improve one or more symptoms of a subject suffering from conditions such as decreased libido, diminishing memory, anemia due to marrow failure, renal failure, chronic respiratory or cardiac failure, steroid-dependent autoimmune disease, muscle wasting associated with various diseases such as AIDS, preventing attacks of hereditary angioedema or urticaria; andropause, and palliating cancer. In some situations, certain biomarkers such as for example, increased SHBG levels, can be used to diagnose a subject who may be in need of testosterone therapy. These biomarkers can be associated with conditions/disease states such as anorexia nervosa, hyperthyroidism, hypogonadism, androgen insensitivity/deficiency, alcoholic hepatic cirrhosis, primary biliary cirrhosis, and the like.

Methods of Using Substantially Pure (17-β)-3-Oxoandrost-4-en-17-yl Undecanoate and Products Derived Therefrom

In one embodiment, a pharmaceutical composition prepared by synthesizing (17-β)-3-Oxoandrost-4-en-17-yl undecanoate to produce substantially pure (17-β)-3-Oxoandrost-4-en-17-yl undecanoate solid and mixing the substantially pure (17-β)-3-Oxoandrost-4-en-17-yl undecanoate with one or more pharmaceutically acceptable carriers to provide a pharmaceutical composition which is substantially free of impurities. In a related embodiment, a pharmaceutical composition is prepared by providing substantially pure (17-β)-3-Oxoandrost-4-en-17-yl undecanoate solid and mixing the substantially pure (17-β)-3-Oxoandrost-4-en-17-yl undecanoate with one or more pharmaceutically acceptable carriers to provide a pharmaceutical composition which is substantially free of impurities.

The compositions and unit dosage forms can be prepared by any suitable method known to the skilled artisan or developed in view of the teachings herein.

In one specific aspect, the carrier(s) and API are brought to or maintained at a temperature at which they are flowable (e.g., above 10° C., 20° C., 25° C., 30° C., 35° C., or 40° C.). In one aspect, the mixture of carrier and API is a clear solution at a specified temperature (e.g., above 10° C., 20° C., 25° C., 30° C., 35° C., or 40° C.). In one aspect, the mixture of carrier and API is a cloudy or hazy solution at a specified temperature (e.g., below 10° C., 20° C., 25° C., 30° C., 35° C., or 40° C.

In one example, the composition is prepared by weighing all of the components, except the API into a clean stainless steel container and mixed together at ambient temperature or at elevated temperatures e.g., at about 25° C. to about 30° C., at about 30° C. to about 35° C., at about 35° C. to about 40° C., at about 40° C. to about 45° C., at about 45° C. to about 45° C., or 50° C. to about 70° C., using a stirrer. The API is added and stirred into the mixture of other components until the API dissolves. A predetermined quantity of this “liquid fill material” is disposed into a capsule (for example, hard gelatin capsule) to get the required API dose per dosage unit. The capsules are allowed to cool at room temperature, banded (if required) and packaged in a HDPE bottle and tightly closed with an appropriate lid. It is noted that various capsule sizes (e.g., hard gel or soft gel) are available to the skilled artisan and allow for variations in the amount of loading of API in mg per unit dosage form. Typically, soft gel capsules for oral administration have fill volumes of less than 1.5 mL, 1.3 mL or 1.25 mL with numerous incremental fill volumes in these ranges. Similarly, hard gel capsules typically have fill volumes of less than 1.25 mL, 1.10 mL or 1 mL. Due to the nature of some hard gel capsules, the total fill volume may not be useable. There is a practical limit on the temperature at which capsules can be filled—for example temperature above 40° C. typically melt, deform, or otherwise damage soft gel capsules typically employed in the industry. Hard gel capsules are typically less sensitive to temperature and can be filled at higher temperatures e.g., above 40° C.

In certain embodiments, any pharmaceutical composition described herein, e.g., a can be prepared by (i) combining and heating all ingredients until a molten mixture is obtained (e.g., 50-70° C.); and (ii) encapsulating an amount of molten mixture comprising a select dose (e.g., a therapeutically effective amount or a partial dose of a therapeutically effective amount) API to obtain an oral dosage form. In certain instances, the molten mixture is spray-congealed to obtain beads. In some instances, the molten mixture is sprayed onto inert cores (e.g., sugar spheres) to obtain coated cores. In certain embodiments, such beads, cores, or similar forms are encapsulated or otherwise formulated to provide an oral dosage form. In some instances, the molten mixture is admixed, uniformly dispersed, or granulated over a carrier and compressed into a tablet dosage form. In certain embodiments, prior to compression, the molten mixture/carrier composition is further mixed with one or more pharmaceutical aid including, by way of non-limiting example, glidants, lubricants, binders, or the like. In some embodiments, the carrier is a therapeutically inert carrier such as, by way of non-limiting example, microcrystalline cellulose, starch, lactose, or the like.

In various embodiments, pharmaceutical compositions described herein are formulated as oral dosage forms. Oral dosage forms are prepared by any suitable process including one or more steps of, by way of non-limiting example, agglomeration, air suspension chilling, air suspension drying, balling, coacervation, comminution, compression, pelletization, cryopelletization, encapsulation, extrusion, granulation, homogenization, inclusion complexation, lyophilization, nanoencapsulation, melting, mixing, molding, pan coating, solvent dehydration, sonication, spheronization, spray chilling, spray congealing, spray drying, or the like.

In some embodiments, a pharmaceutical composition described herein is formulated with a substrate to form an oral dosage form. In various embodiments, substrates useful for formulating pharmaceutical compositions described herein as oral dosage forms include or comprise, by way of non-limiting example, a powder or a multiparticulate (e.g., one or more granule, one or more pellet, one or more bead, one or more spherule, one or more beadlet, one or more microcapsule, one or more millisphere, one or more mini capsule, one or more microcapsule, one or more nanocapsule, one or more nanosphere, one or more microsphere, one or more minitablet, one or more tablet, one or more capsule, or one or more combinations thereof). In certain instances, a powder constitutes a finely divided (milled, micronized, nanosized, precipitated) form of an active ingredient or additive molecular aggregates or a compound aggregate of multiple components or a physical mixture of aggregates of an active ingredient and/or additives.

The following examples are provided to promote a more clear understanding of certain embodiments of the present invention, and are in no way meant as a limitation thereon.

### EXAMPLE 1

### Preparation of Substantially Pure Pharmaceutical Ingredient, (17-β)-3-Oxoandrost-4-en-17-yl Undecanoate

1) (17-β)-Hydroxy-4-Androsten-3-one (0.1 mol) is weighed into a 1000 mL 4N RB flask containing a stir bar;

2) Pyridine (160 mL) is added to the flask;

3) The flask is placed in an ice-water bath and fitted with a nitrogen inlet, addition funnel, thermocouple, and stopper. Stirring and nitrogen flow are started;

4) The funnel is charged with a solution of acid chloride (1.56 equiv e.g., acid chloride of undecanoic acid) in heptane (160 mL), then fitted with an adapter connected to a bubbler;

5) The contents of the funnel are added drop wise over 30-40 min (Note: the internal temperature increases 5-7° C. during the addition);

6) When the addition is complete, the bath is removed and stirring is continued;

7) After 1 h, the reaction mixture is transferred to a large separatory funnel and diluted with heptane (1000 mL) (Note: Thin layer chromatography (“TLC”) can be used to monitor the reaction e.g., after one hour);

8) The heptane solution is washed successively with 800 mL portions of: cold water (2×), 0.05 N NaOH, saturated NaHCO₃ (2×), water, brine, then dried over anhydrous Na₂SO₄ (−50 g). Then concentrated to dryness (rotavap/Tbath:S; 30° C.).

### EXAMPLE 2

### Preparation of Substantially Pure API Crystals

A reaction mixture of Example 1 can be transferred to water, ethanol, or methanol (or any other suitable solvent) and allowed to crystallize. The crystalline mass can be filtered by suction, washed with water, dried over phosphorous pentoxide and re-crystallized from another solvent e.g., oleic acid, hexane, heptanes, etc.

### EXAMPLE 3

### Purification of (17-β)-3-Oxoandrost-4-en-17-yl Undecanoate Pharmaceutical Ingredient by Liquid Chromatography

Liquid chromatography can be used to purify or analyze the purity of samples having (17-β)-3-Oxoandrost-4-en-17-yl undecanoate

One exemplary liquid chromatographic method is as follows.

Column: size 0.3 m, diameter 4.0 mm; stationary phase—end-capped octadecylsilyl silica gel for chromatography (5 micron); Temperature 40 C.

Mobile Phase water: acetonitrile (5:95 VAT)

Flow rate 1.0 mL/min

Detector—240 nm

Dissolve sample in mobile phase (e.g., 20 mg in 50 mL).

Injection: 20 uL.

Run time—twice the retention time of (17-β)-3-Oxoandrost-4-en-17-yl undecanoate.

Order of retention time of impurities and (17-β)-3-Oxoandrost-4-en-17-yl undecanoate starting with first eluting is expected as follows:
(1) Undecanoate;
(2) (17-β)-3-Oxoandrost-4-en-3-one;
(3) (17-β)-3-Oxoandrost-4-en-17-yl decanoate;
(4) (17-β)-3-Oxoandrost-4-en-17-yl undecanoate;
(5) (17-β)-3-Oxoandrost-4-en-17-yl dodecanoate;
(6) (17-β)-3-Oxoandrost-4-en-17-yltridecanoate; and
(7) (17-β)-3-Oxoandrost-4-en-17-yltetradecanoate,

According to this procedure, these impurities(and others) can be analyzed and identified (and others described herein in this Specification and Figures). For example, mass spectrometry in conjunction with liquid chromatography can be used to assess or identify impurities in a sample having (17-β)-3-Oxoandrost-4-en-17-yl undecanoate,

### EXAMPLE 4

### Separation of Impurities

Impurities in (17-β)-3-Oxoandrost-4-en-17-yl undecanoate or composition containing (17-β)-3-Oxoandrost-4-en-17-yl undecanoate can be separated and/or identified according to the following procedure

Column: C18

Mobile Phase: acetonitrile; alcohol; and deionized water.

Detector Wavelength: about 240 nm (e.g., 242 nm)

Flow Rate: 1.0 mL per minute

Column Temperature: 29° C.

A sample used derived from a (17-β)-3-Oxoandrost-4-en-17-yl undecanoate formulation/composition stored at selected temperatures (e.g., ambient, 25, 30, 35, 40, 50, 60° C. or more) and for selected amounts of time (e.g., 0, 1, 2, 3, 4, 5, 6, 7, 8, 9, 12, 15, 18 or 24 months can be used to assess impurities (and/or stability) by HPLC. Placebo labeled peaks can be identified from a similar formulations not having (17-β)-3-Oxoandrost-4-en-17-yl undecanoate. The formulation in this Example is as follows:

Composition (99)

| | Weight Percent of | Quantity Fill |
|---|---|---|
| | Fill Pharmaceutical | Material per Hard |
| | Composition (±1%) | Gel Capsule (±1%) |
| Ingredient Name | % w/w | mg |
| API | 15 | 110 |
| Glyceryl Monolinoleate, NF | 63 | 463 |
| Polyoxyl 40 Hydrogenated | 15 | 114 |
| Castor Oil, NF | | |
| Ascorbyl Palmitate, NF | 0.2 | 1.5 |
| Polyethylene Glycol 8000, NF | 6 | 44 |
| Total | 100 | 733.3 |

It is believed that formulations without a stabilizing agent e.g., antioxidant (e.g., ascorbyl palmitate) had the buildup over time of the impurities putatively identified as hydroxylated at position 6 of the compound in FIG. 6 in the “out of the plane”, “in the plane”, or both. For example after two years storage of the composition without ascorbyl palmitate up to about 0.5% of these compounds could form.

### EXAMPLE 5

### Identification of Impurities by LC/MS

Samples related to (17-β)-3-Oxoandrost-4-en-17-yl undecanoate can be analyzed by LC/MS. In one specific example, substantial pure (17-β)-3-Oxoandrost-4-en-17-yl undecanoate is exposed to elevated temperatures for 24 hours or 48 hours. The samples are dissolved in a solvent after exposure to elevated temperatures. The samples are then prepared for the LC/MS experiments by a 1:10,000 dilution into (20:80 acetonitrile:distilled/deionized water) by first dilution into the acetonitrile and then by bringing the volume up to the final volume by dilution with the distilled/deionized water.

### EXAMPLE 6

### Stability of API Described Herein

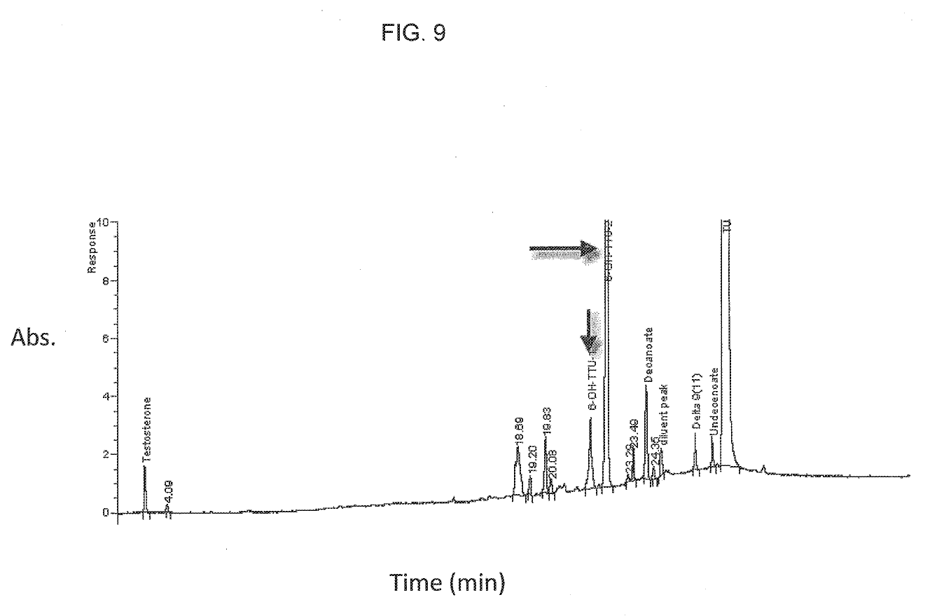

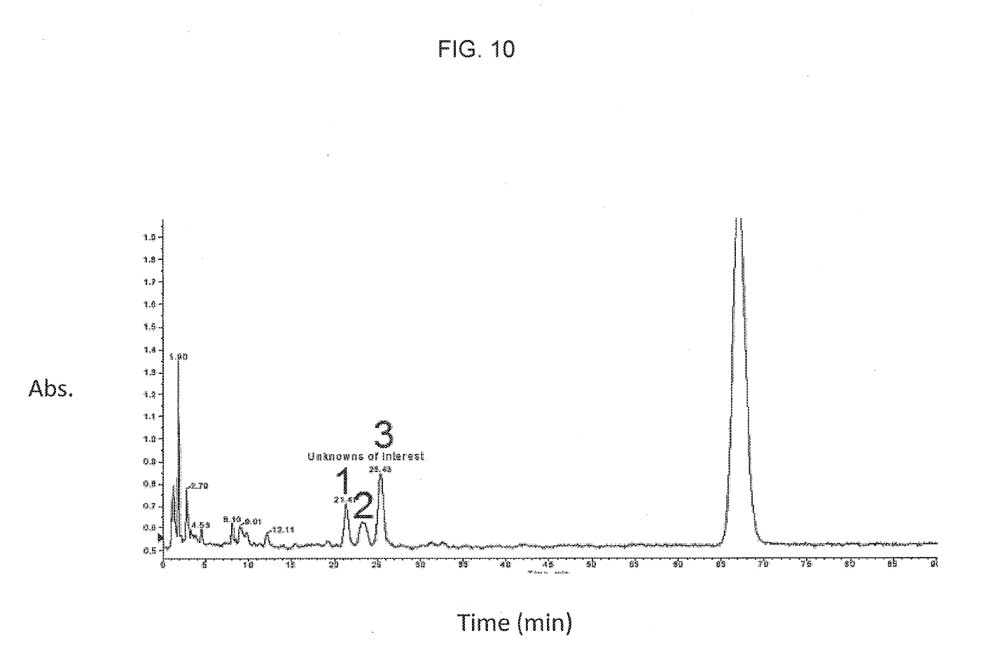

The HPLC traces in FIGS. 9 and 10 are from samples of API that were subjected to forced degradation conditions e.g., high temperature (105° C.), acidic conditions, oxidizing environments etc.

Exemplary HPLC conditions are a C18 column (5 μm), 150×3.9 mm, 90% methano1:10% deionized water at a flow rate of 1.0 mL/min with the column at 25° C. or as described elsewhere.

### EXAMPLE 7

### Release Profile

The compositions, dosage forms described herein containing API can subjected to in vitro dissolution (release) testing using USP type 2 apparatus in about 1000 mL aqueous medium. The composition (e.g., dosage form) is subjected to in vitro dissolution testing using USP type 2 apparatus in about e.g., 1000 mL 8% Triton X100 solution in water at a specific temperature (e.g., 37° C.) at 100 rpm for a specific time (e.g., 1, 2, 3, 4, 5, 10, 15, 30, 45, 60, 75, 90, 120, 180, or 240 minute time point where a sample is withdrawn and analyzed for API content (e.g., via HPLC)).

### EXAMPLE 8

### Release Profile Stability

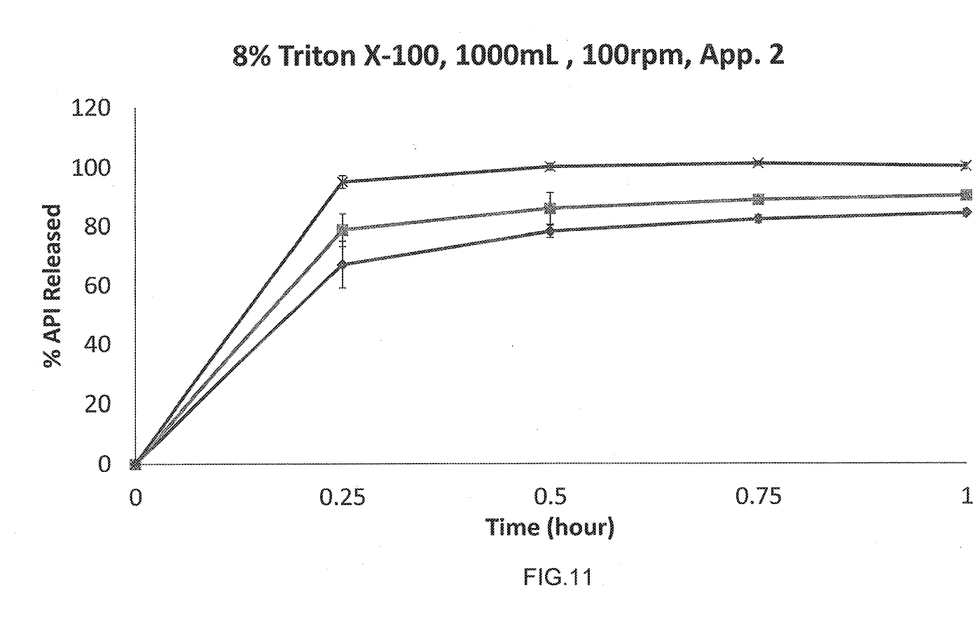

The compositions, dosage forms described herein containing API can subjected to in vitro dissolution (release) testing using USP type 2 apparatus in about 1000 mL aqueous medium as described in the above example after storage for particular amounts of time under specific conditions. FIG. 11 shows the release profile stability of the composition below (e.g., unit dosage form of composition described herein in this example). The center line represents time point 0; 1 month storage at 25° C. and 60% relative humidity and 1 month storage at 40° C. and 75% relative humidity (all three sample overlaid); the bottom line solid line represents 2 month storage at 25° C. and 60% relative humidity; and the top line represents 2 month storage at 40° C. and 75% relative humidity.

Composition (200).

| | Theoretical Qty. | |
|---|---|---|
| | per Capsule | |
| Ingredient Name | % w/w | mg |
| (17-β)-3-Oxoandrost-4-en-17-yl undecanoate | 30.00 | 150.0 |
| Oleic Acid, NF | 53.80 | 269.0 |
| Polyoxyl 40 Hydrogenated Castor Oil, NF | 4.00 | 20.0 |
| Stearic Acid, NF | 4.00 | 20.0 |
| Glyceryl Palmitostearate (Glyceryl Distearate, NF; | 8.00 | 40.0 |
| Precirol ATO 5) | | |
| Ascorbyl Palmitate, NF | 0.20 | 1.0 |
| Total | 100.00 | 500.0 |

Processing temperature target can be 48° C.

Filling temperature target can be 38° C.

The amount of loading of (17-β)-3-Oxoandrost-4-en-17-yl undecanoate can be changed as well as the amount of capsule fill material. These formulations allow for dosing of e.g., 150 mg of API or 225 mg of API in one unit dosage form.

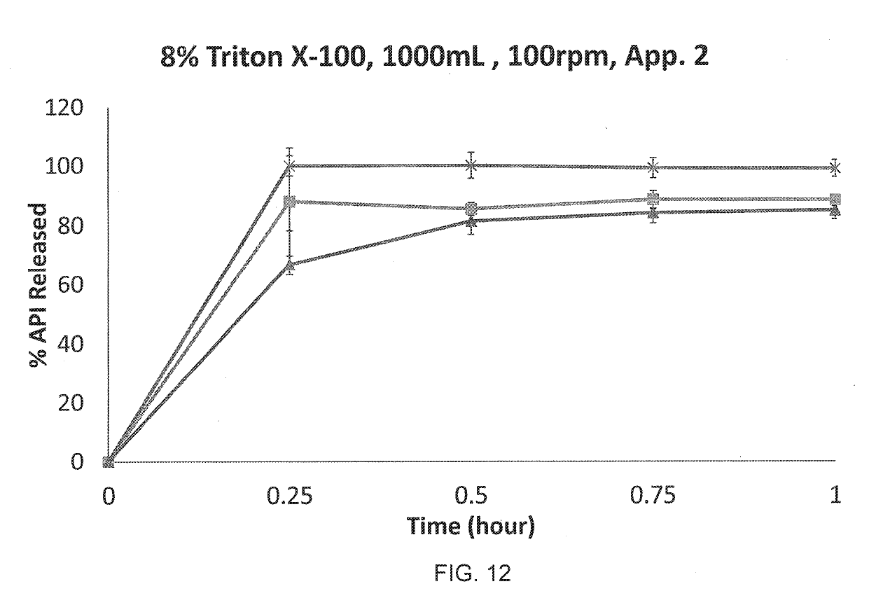

FIG. 12 shows the release profile stability for the composition (e.g., unit dosage form) having 225 mg API at time 0 (middle line), 1 month stored at either 25° C. 60% RH (bottom line) or 40 ° C. 75% RH (top line).

The 150 mg Formulation and 225 mg formulation are as in the formulation above in this Example (Composition (200)).

This table shows the amount of testosterone formed at the indicated time and conditions.

| | | | | T = 1 month | | T = 2 months | |
|---|---|---|---|---|---|---|---|
| Formulation | | TU % | T = 0 | 25° C./ | 40° C./ | 25° C./ | 40° C./ |
| Code | | Load | Ambient | 60% RH | 75% RH | 60% RH | 75% RH |
| 150 | mg API | 30% | 0.05% | 0.08% | 0.48% | 0.12% | 0.82% |
| 225 | mg API | 30% | 0.05% | 0.09% | 0.41% | 0.17% | 0.95% |
| 148.5 | mg | 19.8% | <0.05% | 0.20% | 0.90% | NA | NA |

This Table shows the amount of related compound peaks at the indicated times and conditions as assessed by HPLC. RRT is relative retention time to API.

| | | RC | | T = 1 month | | T = 2 months | |
|---|---|---|---|---|---|---|---|
| Formulation | TU % | Peak | T = 0 | 25° C./ | 40° C./ | 25° C./ | 40° C./ |
| Code | Load | RRT | Ambient | 60% RH | 75% RH | 60% RH | 75% RH |
| 150 mg | 30% | 0.34 | <0.05% | ND * | <0.05% * | <0.05% * | ND * |
| | | 0.38 | ND | <0.05% * | <0.05% * | ND * | ND * |
| | | 0.41 | <0.05% | <0.05% * | 0.05% * | 0.08% * | 0.07% * |
| Total Unknown RC | | | 0.06% | 0.04% * | 0.08% * | 0.10% * | 0.07% * |
| 225 mg | 30% | 0.34 | <0.05% | <0.05% * | <0.05% * | <0.05% * | <0.05% * |
| | | 0.38 | ND | ND * | ND * | ND * | ND * |
| | | 0.41 | <0.05% | 0.05% * | 0.05% * | 0.08% * | 0.08% * |
| Total Unknown RC | | | 0.05% | 0.07% * | 0.07% * | 0.10% * | 0.10% * |
| 148.5 mg | 19.8% | 0.34 | <0.05% | <0.05% | <0.05% | NA | NA |
| | | 0.38 | 0.05% | ND | ND | NA | NA |
| | | 0.41 | 0.11% | 0.14% | 0.17% | NA | NA |

| | | | | | |
|---|---|---|---|---|---|
| Total Unknown RC | 0.19% | 0.18% | 0.21% | NA | NA |

The 148.5 mg composition is as follows.

| | | | Theoretical |
|---|---|---|---|
| | Theoretical Qty. | | Qty. for 100 |
| | per Capsule | | Capsules |
| Ingredients Name | % w/w | mg | Target, g |
| (17-β)-3-Oxoandrost-4-en-17-yl | 19.8 | 148.5 | 14.85 |
| undecanoate | | | |
| Oleic Acid, NF | 51.6 | 387.0 | 38.7 |
| Polyoxyl 40 Hydrogenated Castor Oil, | 16.1 | 120.75 | 12.075 |
| NF | | | |
| Borage Seed Oil, NF | 10.0 | 75.0 | 7.5 |
| Peppermint Oil, NF | 2.5 | 18.75 | 1.875 |
| Beta-hydroxy-toluene (BHT), NF | 0.03 | 0.225 | 0.0225 |
| Total | 100.00 | 750.225 | 75.02 |

The table below represents storage of composition containing API for the indicated time under the indicated compositions. The samples were then assessed by HPLC and the relative retention times of impurities in the samples assessed.

(75 mg API capsule, 15% drug loading, with desiccant)

| | 6 months | | 9 months | |
|---|---|---|---|---|
| Condition | RRT 0.37 | RRT 0.40 | RRT 0.37 | RRT 0.40 |
| 25° C./60% RH | 0.13 | ND | 0.12 | 0.11 |
| 30° C./65% RH | 0.13 | 0.13 | 0.11 | 0.16 |
| 40° C./75% RH | 0.13 | 0.24 | — | — |

(50 mg API capsule, 15% drug loading, without desiccant)

| | 6 months | | 12 months | |
|---|---|---|---|---|
| Condition | RRT 0.37 | RRT 0.40 | RRT 0.37 | RRT 0.40 |
| 25° C./60% RH | 0.14 | ND | 0.29 | 0.13 |
| 30° C./65% RH | 0.27 | 0.14 | 0.39 | 0.27 |
| 40° C./75% RH | 0.7 | 0.44 | — | — |

The composition used in regards to the above 2 tables is as below.

Composition (99A)

| | Weight Percent of |
|---|---|
| | Fill Pharmaceutical |
| | Composition (±1%) |
| Ingredient Name | % w/w |
| API | 15 |
| Glyceryl Monolinoleate, NF | 63 |
| Polyoxyl 40 Hydrogenated Castor Oil, NF | 15 |
| Ascorbyl Palmitate, NF | 0.2 |
| Polyethylene Glycol 8000, NF | 6 |
| Total | 100 |

### EXAMPLE 9

### Pharmaceutical Compositions

Shown below are various compositions suitable for oral administration as described herein. In these Examples the amount of excipient adds up to 100% (does not include the API) and the API weight percent is the final weight percent in the pharmaceutical composition.

| | Composition No. | | | | | | |
|---|---|---|---|---|---|---|---|
| Component (w/w %) | 1 | 2 | 3 | 4 | 5 | 6 | 7 |
| API | 22 | 23 | 24 | 26 | 28 | 30 | 32 |
| Excipient 1 | 35-80 | 35-80 | 35-80 | 35-80 | 35-80 | 35-80 | 35-80 |
| (e.g., liquid | | | | | | | |
| carrier) | | | | | | | |
| Excipient 2 | 1-40 | 1-40 | 1-40 | 1-40 | 1-40 | 1-40 | 1-40 |
| (e.g., additive) | | | | | | | |
| Excipient 3 | 0-20 | 0-20 | 0-20 | 0-20 | 0-20 | 0-20 | 0-20 |
| (e.g., hydrophilic | | | | | | | |
| additive) | | | | | | | |
| Excipient 4 | 0.01-3 | 0.01-3 | 0.01-3 | 0.01-3 | 0.01-3 | 0.01-3 | 0.01-3 |
| (e.g., anti-oxidant) | | | | | | | |
| Additional | qs | qs | qs | qs | qs | qs | qs |
| Excipients | | | | | | | |
| (e.g., other | | | | | | | |
| pharmaceutically | | | | | | | |
| acceptable | | | | | | | |
| excipients ) | | | | | | | |

The API in this example in specific compositions is ((17-β)-3-Oxoandrost-4-en-17-yl undecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid; H—(o—CH₂—CH₂)_{N}—OH where n is an integer from 3 to 900; octadecanoic acid; (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene; or a combination thereof. Excipient 3 in specific compositions is a polyoxylated hydrogenated vegetable oil. Excipient 4 in specific compositions is ascorbyl palmitate. These compositions can be filled into soft gel or hard gel capsules depending on its flowability at the temperatures useful for making these dosage forms.

### EXAMPLE 10

### Pharmaceutical Compositions

In these Examples the amount of excipient adds up to 100% (does not include the API) and the API weight percent is the final weight percent in the pharmaceutical composition.

| | Composition No. | | | | | | | | |
|---|---|---|---|---|---|---|---|---|---|
| Component (%) | 10 | 11 | 12 | 13 | 14 | 15 | 16 | 17 | 18 |
| API | 23 | 24 | 25 | 26 | 27 | 28 | 29 | 30 | 31 |
| Excipient 1 | 40-70 | 30-70 | 40-70 | 40-70 | 40-70 | 40-70 | 40-70 | 30-70 | 40-70 |
| (e.g., C14-C20 | | | | | | | | | |
| fatty acid) | | | | | | | | | |
| Excipient 2 | 0.5-20 | | | 1-20 | | | 1-20 | | |
| (e.g., glyceryl | | | | | | | | | |
| palmitostearate) | | | | | | | | | |
| Excipient 3 | | 0.5-30 | | | 5-35 | | | 10-30 | |
| Excipient 4 | | | 0.5-15 | | | 1-12 | | | 2-11 |
| (e.g., polyethylene | | | | | | | | | |
| glycol (high | | | | | | | | | |
| molecular weight)) | | | | | | | | | |
| Excipient 4 | 0.01-3 | 0.01-3 | 0.01-3 | 0.01-3 | 0.01-3 | 0.01-3 | 0.01-3 | 0.01-3 | 0.01-3 |
| (e.g., anti-oxidant | | | | | | | | | |
| (ascorbyl palmitate) | | | | | | | | | |
| Additional | qs | qs | qs | qs | qs | qs | qs | qs | qs |
| Excipients | | | | | | | | | |

The API in this example in specific compositions is ((17-β)-3-Oxoandrost-4-en-17-yl undecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid or a combination thereof. Excipient 3 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene.

Excipient 4 in specific compositions is H—(O—CH₂—CH₂)ₙ—OH where n is an integer from 3 to 900 (e.g., PEG having an average molecular weight in the range of 2000-12000). These compositions can be filled into soft gel or hard gel capsules depending on its flowability at the temperatures useful for making these dosage forms. These compositions may include a hydrophilic additive.

### EXAMPLE 11

### Pharmaceutical Compositions

In these Examples the amount of excipient adds up to 100% (does not include the API) and the API weight percent is the final weight percent in the pharmaceutical composition.

| | Composition No. | | | | | | | | |
|---|---|---|---|---|---|---|---|---|---|
| Component | 19 | 20 | 21 | 22 | 23 | 24 | 25 | 26 | 27 |
| API | 23 | 24 | 25 | 26 | 27 | 28 | 29 | 30 | 31 |
| Excipient 1 | 40-90 | 40-90 | 40-90 | 40-90 | 40-90 | 40-90 | 40-90 | 40-90 | 40-90 |
| Excipient 2 | 1-20 | | 1-20 | | 1-20 | | 1-20 | | 1-20 |
| Excipient 3 | | 1-10 | | 1-10 | | 1-10 | | 1-10 | |
| Excipient 4 | 0-25 | 0-25 | 0-25 | 0-25 | 0-25 | 0-25 | 0-25 | 0-25 | 0-25 |
| Additional | qs | qs | qs | qs | qs | qs | qs | qs | qs |
| Excipients | | | | | | | | | |

The API in this example in specific compositions is (17-β)-3-Oxoandrost-4-en-17-yl undecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid or a combination thereof. Excipient 3 in specific compositions is H—(O—CH₂—CH₂)ₙ—OH where n is an integer from 3 to 900(e.g., PEG having an average molecular weight in the range of 2000-12000) . Excipient 4 in specific compositions is (1R,2S,5R)-2-isopropyl-5-methylcyclohexanol or a combination of one or more of (1R,2S,5R)-2-isopropyl-5-methylcyclohexanol, (2 S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. These compositions can be filled into soft gel or hard gel capsules depending on its flowability at the temperatures useful for making these dosage forms.

### EXAMPLE 12

### Pharmaceutical Compositions

In these Examples the amount of excipient adds up to 100% (does not include the API) and the API weight percent is the final weight percent in the pharmaceutical composition.

| | # | | | | | | | | |
|---|---|---|---|---|---|---|---|---|---|
| Component | 28 | 29 | 30 | 31 | 32 | 33 | 34 | 35 | 36 |
| API (%) | 23 | 24 | 25 | 26 | 27 | 28 | 29 | 30 | 31 |
| Excipient 1 | 40-90 | 45-90 | 50-90 | 55-90 | 40-90 | 45-90 | 55-90 | 50-90 | 50-90 |
| Excipient 2 | 5-15 | 1-15 | 5-15 | 1-15 | | | | | 5-15 |
| Excipient 3 | | | | | 1-10 | 1-10 | 1-10 | 1-10 | |
| Excipient 4 | 0-25 | 0-25 | 0-25 | 0-25 | 0-25 | 0-25 | 0-25 | 0-25 | 0-25 |
| Additional | qs | qs | qs | qs | qs | qs | qs | qs | qs |
| Excipients | | | | | | | | | |

The API in this example in specific compositions is (17-β)-3-Oxoandrost-4-en-17-yl undecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid, or a combination thereof.

Excipient 3 in specific compositions is H—(O—CH₂—CH₂)ₙ—OH where n is an integer from 3 to 900 (e.g., PEG having an average molecular weight in the range of 2000-12000) . Excipient 4 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. These compositions can be filled into soft gel or hard gel capsules depending on its flowability at the temperatures useful for making these dosage forms.

### EXAMPLE —

### Pharmaceutical Compositions

In these Examples the amount of excipient adds up to 100% (does not include the API) and the API weight percent is the final weight percent in the pharmaceutical composition.

| | Composition No. | | | | | | | | |
|---|---|---|---|---|---|---|---|---|---|
| Component (%) | 37 | 38 | 39 | 40 | 41 | 42 | 43 | 44 | 45 |
| API | 23 | 24 | 25 | 26 | 27 | 28 | 29 | 30 | 31 |
| Excipient 1 | 45-80 | 45-80 | 45-80 | 45-80 | 45-80 | 45-80 | 45-80 | 45-80 | 45-80 |
| (e.g., Fatty | | | | | | | | | |
| acid) | | | | | | | | | |
| Excipient 2 | 1-15 | 1-15 | 1-15 | 1-15 | 1-15 | 1-15 | 1-15 | 1-15 | 1-15 |
| Excipient 3 | 0-10 | 0-10 | 0-10 | 0-10 | 0-10 | 0-10 | 0-10 | 0-10 | 0-10 |
| Excipient 4 | 0.1-0.3 | 0.1-0.3 | 0.1-0.3 | 0.1-0.3 | 0.1-0.3 | 0.1-0.3 | 0.1-0.3 | 0.1-0.3 | 0.1-0.3 |
| Additional | qs | qs | qs | qs | qs | qs | qs | qs | qs |
| Excipients | | | | | | | | | |

The API in this example in specific compositions is (17-β)-3-Oxoandrost-4-en-17-yl undecanoate. Excipient 1 in specific compositions is (9Z)-octadec-9-enoic acid, hexadecanoic acid or a combination thereof. Excipient 2 in specific compositions is a combination of mono-, di-, or tri-propane-1,2,3-triol esters of octadecanoic acid and hexadecanoic acid. Excipient 3 in specific compositions polyoxylated hydrogenated castor oil (Cremophor R40). Excipient 4 in specific compositions is ascorbyl palmitate. These compositions can be filled into soft gel or hard gel capsules depending on its flowability at the temperatures useful for making these dosage forms.

### EXAMPLE 14

### Pharmaceutical Composition, Formulations and Unit Dosage Forms

The API in this example in specific compositions is (17-β)-3-Oxoandrost-4-en-17-yl undecanoate.

These compositions can be made by any suitable method and filled into hard gel or soft gel capsules as appropriate. For example, the one or more of the ingredients are warmed or heated to a temperature that allows for dissolving any solid ingredients, the API is added and mixed until a homogenous mixture is obtained and the capsule can be filled at an appropriate temperature and if needed, allowed to cool to room temperature.

Composition (100)

| | Quantity Fill | |
|---|---|---|
| | Material per Hard | |
| | Shell Capsule | |
| Ingredient Name | % w/w | mg |
| API | 23%-28% | 140-275 |
| Oleic Acid, NF ((9Z)-octadec-9-enoic acid) | 60%-70% | 450-530 |
| Polyoxyl 40 Hydrogenated Castor Oil, NF | 0%-7% | 0-37 |
| Ascorbyl Palmitate, NF | 0.1%-0.3% | 0.5-2.5 |
| Polyethylene Glycol 8000, NF | 3%-9% | 35-55 |
| Total | 100.0 | Adjustable |

Composition (101)

| | Quantity Fill | | Quantity Fill |
|---|---|---|---|
| | Material per Hard | | Material per Soft |
| | Gel Capsule | | Gel Capsule |
| Ingredient Name | % w/w | mg | mg |
| API | 23%-28% | 140-275 | 140-275 |
| Oleic Acid, NF ((9Z)-octadec- | 37%-46% | 280-330 | 532-627 |
| 9-enoic acid) | | | |
| Peppermint Oil, NF | 15-21 | 120-150 | 228-285 |
| Polyoxyl 40 Hydrogenated | 0-7% | 0-35 | 0-67 |
| Castor Oil, NF | | | |
| Ascorbyl Palmitate, NF | 0.20 | 0.5-2.5 | 1.0-4.8 |
| Glyceryl Palmitostearate | 9%-15% | 80-100 | 152-190 |
| (Glyceryl Distearate, NF) | | | |
| Total | 100.0 | Adjustable | Adjustable |

Composition (102)

| | Quantity Fill | | Quantity Fill |
|---|---|---|---|
| | Material per Hard | | Material Soft |
| | Gel Capsule | | Gel Capsule |
| Ingredient Name | % w/w | mg | mg |
| API | 25%-32% | 140-275 | 140-275 |
| Oleic Acid, NF ((9Z)-octadec- | 50%-60% | 340-400 | 646-760 |
| 9-enoic acid) | | | |
| Polyoxyl 40 Hydrogenated | 0%-7% | 21-32 | 0-61 |
| Castor Oil, NF | | | |
| Stearic Acid, NF | 0%-7% | 0-32 | 0-61 |
| Glyceryl Palmitostearate | 3%-13% | 47-58 | 89-110 |
| (Glyceryl Distearate, NF; | | | |
| Precirol ATO 5) | | | |
| Ascorbyl Palmitate, NF | 0.1%-3% | 0.5-2.5 | 1.0-4.8 |
| Total | 100.00 | adjustable | adjustable |

Composition (103)

| | Quantity Fill | |
|---|---|---|
| | Material per Hard | |
| | Gel Capsule | |
| Ingredient Name | % w/w | mg |
| API | 27%-33% | 140-275 |
| Oleic Acid, NF ((9Z)-octadec-9-enoic acid) | 50%-70% | 335-395 |
| Polyoxyl 40 Hydrogenated Castor Oil, NF | 0%-7% | 0-30 |
| Ascorbyl Palmitate, NF | 0.1%-0.3% | 0.5-2.5 |
| Polyethylene Glycol 8000, NF | 3%-9% | 32-42 |
| Total | 100.0 | adjustable |

### EXAMPLE 15

The API in this example in specific compositions is (17-β)-3-Oxoandrost-4-en-17-yl undecanoate.

These compositions can be made by any suitable method and filled into hard gel or soft gel capsules as appropriate. For example, the one or more of the ingredients are warmed or heated to a temperature that allows for dissolving any solid ingredients, the API is added and mixed until a homogenous mixture is obtained and the capsule can be filled at an appropriate temperature and if needed, allowed to cool to room temperature.

Composition (104)

| | Weight Percent of |
|---|---|
| | Fill Pharmaceutical |
| | Composition (±1%) |
| Ingredient Name | % w/w |
| API | 24 |
| Oleic Acid, NF ((9Z)-octadec-9-enoic acid) | 65 |
| Polyoxyl 40 Hydrogenated Castor Oil, NF | 4 |
| Ascorbyl Palmitate, NF | 0.2 |
| Polyethylene Glycol 8000, NF | 6 |
| Total | 100 |

Composition (105)

| | Weight Percent of |
|---|---|
| | Fill Pharmaceutical |
| | Composition (±1%) |
| Ingredient Name | % w/w |
| API | 24 |
| Oleic Acid, NF ((9Z)-octadec-9-enoic acid) | 41 |
| Peppermint Oil, NF | 18 |
| Polyoxyl 40 Hydrogenated Castor Oil, NF | 4 |
| Ascorbyl Palmitate, NF | 0.2 |
| Glyceryl Palmitostearate (Glyceryl Distearate, NF) | 12 |
| Total | 100 |

Composition (106)

| | Weight Percent of |
|---|---|
| | Fill Pharmaceutical |
| | Composition (±1%) |
| Ingredient Name | % w/w |
| API | 28 |
| Oleic Acid, NF ((9Z)-octadec-9-enoic acid) | 55 |
| Polyoxyl 40 Hydrogenated Castor Oil, NF | 4 |
| Stearic Acid, NF | 4 |
| Glyceryl Palmitostearate (Glyceryl Distearate, NF; | 8 |
| Precirol ATO 5) | |
| Ascorbyl Palmitate, NF | 0.2 |
| Total | 100 |

Composition (107)

| | Weight Percent of |
|---|---|
| | Fill Pharmaceutical |
| | Composition (±1%) |
| Ingredient Name | % w/w |
| API | 30 |
| Oleic Acid, NF ((9Z)-octadec-9-enoic acid) | 59 |
| Polyoxyl 40 Hydrogenated Castor Oil, NF | 4 |
| Ascorbyl Palmitate, NF | 0.2 |
| Polyethylene Glycol 8000, NF | 6 |
| Total | 100 |

Composition (108)

| | Theoretical Qty. |
|---|---|
| | per Capsule |
| Ingredient Name | % w/w |
| (17-β)-3-Oxoandrost-4-en-17-yl undecanoate | 23-35 |
| Oleic Acid, NF ((9Z)-octadec-9-enoic acid) | 40-70 |
| Polyoxyl 40 Hydrogenated Castor Oil, NF | 0-10 |
| Stearic Acid, NF | 0-10 |
| Glyceryl Palmitostearate (Glyceryl Distearate, NF; | 1-15 |
| Precirol ATO 5) | |
| Ascorbyl Palmitate, NF | 0.1-3 |
| Total | 100.00 |

As is apparent to the skilled artisan, these formulations can be varied and still yield the beneficial properties described herein. In a particular example, of these compositions and those described throughout this specification are formulations having from 140-160 mg (e.g., 150 mg) or 215 to 235 mg (e.g., 225) mg of API (or 300 mg, 112.5 mg or 75 mg API) which are particularly preferred and similar in composition to those described in Compositions (101)-(108).

Based on the description provided herein and the results of the clinical trial, it is now possible to provide pharmaceutical compositions similar to those described as Compositions (A)-(J) having API ((8R,9S,10R,13S,14S,17S)-10,13-dimethyl-3-oxo-1,2,6,7,8,9,11,12,14,15,16,17-dodecahydrocyclopenta[a]phenanthren-17-yl undecanoate in an amount of e.g., 60 mg to 85 mg, 85 mg to 100 mg, 100 mg to 115 mg, 115 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 525 mg to 550 mg, 550 mg to 575 mg, or575 mg to 600 mg. Similar composition to Compositions (101)-(108) to those above can also have for example:
(a) a different fatty acid, an additional fatty acid or a booth,
(b) a different hydrophilic surfactant, an additional hydrophilic surfactant or both,
(c) a mono- or di-glyceride in place of the fatty acid or in combination with the fatty acid,
(d) a different solidifying agent, an additional solidifying agent, or both,
(e) a different diglyceride than glyceryl palmitostearate, an additional diglyceride or both,
(f) a different antioxidant, an additional antioxidant or both,
(g) have additional additives,
(h) use menthol or another alcohol in place of or in addition to peppermint oil,
(i) use a tocopherol in place of fatty acid, in combination with fatty acid, in place of peppermint oil, in addition to peppermint oil or a combination thereof,
(j) use a different monoglyceride than glyceryl monolinoleate, an additional monoglyceride, a diglyceride in place of glyceryl monolinoleate, a diglyceride in combination with glyceryl monolinoleate or a combination thereof, or
(k) a combination of any of the above.

Preferably the composition is bioequivalent to composition (99) or (99A) where the composition (99) or (99A) has about the same percent amount of excipients and either 75 mg, 112.5 mg, 225 mg, or 300 mg of API. In one aspect, the API can be loaded at any weight percent that is sufficient to provide the same bioavailability of Composition (99) or (99A) having about the same percent amount of excipients and either 75 mg, 112.5 mg, 225 mg, or 300 mg of API

Similarly, different amounts of the excipients and different percent loading of the API may be used.

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

1. Substantially pure (17-β)-3-Oxoandrost-4-en-17-yl undecanoate suitable for administration to human subject in need of 17 beta-hydroxy androst-4-en-3-one.

2. The substantially pure (17-β)-3-Oxoandrost-4-en-17-yl undecanoate of claim 1 that has greater than 90% potency with respect to (17-β)-3-Oxoandrost-4-en-17-yl undecanoate.

3. The substantially pure (17-β)-3-Oxoandrost-4-en-17-yl undecanoate of claim 1 that is substantially free of impurities.

4. The substantially pure (17-β)-3-Oxoandrost-4-en-17-yl undecanoate of claim 3 having 10% or less of total impurities.

5. The substantially pure (17-β)-3-Oxoandrost-4-en-17-yl undecanoate of claim 4 having 10% or less of total known impurities.

6. The substantially pure (17-β)-3-Oxoandrost-4-en-17-yl undecanoate of claim 4 having 10% or less of total unknown impurities.

7. The substantially pure (17-β)-3-Oxoandrost-4-en-17-yl undecanoate of claim 4 having 10% or less of any single known impurity.

8. The substantially pure (17-β)-3-Oxoandrost-4-en-17-yl undecanoate of claim 7 having 10% or less of beta-hydroxy androst-4-en-3-one.

9. The substantially pure (17-β)-3-Oxoandrost-4-en-17-yl undecanoate of claim 6 having 2% or less of any single unknown impurity.

10. The substantially (17-β)-3-Oxoandrost-4-en-17-yl undecanoate of claim 1 comprising less than 50,000 PPM organic solvent.

11. The human subject of claim 1 wherein the human is a male.

12. The male of claim 11 is a hypogonadal male.

13. A pharmaceutical composition comprising or made from a substantially pure (17-β)-3-Oxoandrost-4-en-17-yl undecanoate and a pharmaceutically acceptable carrier for administration to human subject in need of (17-β)-hydroxy-4-androsten-3-one.

14. The pharmaceutical composition of claim 13 suitable for dosing up to 2000 mg per day (17-β)-3-Oxoandrost-4-en-17-yl undecanoate to an individual in need of (17-β)-hydroxy-4-androsten-3-one in single or divided doses.

15. The pharmaceutical composition of claim 13 which is a liquid, solution, suspension, dispersion, solid, semi-solid, a gel, a lotion, paste, granule, aggregate, powder, foam, spray, emulsion, syrup, or ointment.

16. The pharmaceutical composition of claim 13 further comprising a stabilizing agent.

17. The pharmaceutical composition of claim 13 further comprising a pharmaceutically is selected from group consisting of acid, base, antioxidant, or desiccant.

18. The pharmaceutical composition of claim 13 for formulated for oral administration.

19. The pharmaceutical composition of claim 13, wherein the composition is substantially free of a decomposition product of (17-β)-3-Oxoandrost-4-en-17-yl undecanoate resulting from oxidation, reduction, cleavage of the ester moiety; oxidation of the steroid ring system; cleavage of one or more rings of the steroid ring system; rearrangement of the steroid ring;
dealkylation of the steroid ring; dealkylation of the ester; or a combination thereof

20. The pharmaceutical composition of claim 13, wherein the potency of the substantially pure (17-β)-3-Oxoandrost-4-en-17-yl undecanoate is retained to at least 90% of the initial amount added to the composition.

21-39. (canceled)
